(19) United States
(12) Patent Application Publication
(10) Pub. No.: US 2019/0345542 A1
(43) Pub. Date: Nov. 14, 2019

(54) RAPID ANTIMICROBIAL SUSCEPTIBILITY TESTING AND PHYLOGENETIC IDENTIFICATION

(71) Applicant: HELMHOLTZ-ZENTRUM FÜR INFEKTIONSFORSCHUNG GMBH, Braunschweig (DE)

(72) Inventors: Susanne HÄUSSLER, Braunschweig (DE); Sebastian BRUCHMANN, Braunschweig (DE); Delphine CHESNEL, Hanover (DE)

(21) Appl. No.: 16/477,794

(22) PCT Filed: Jan. 15, 2018
(86) PCT No.: PCT/EP2018/050890

§ 371 Date: Jul. 12, 2019

(30) Foreign Application Priority Data
Jan. 13, 2017 (EP) 17151531.5

(51) Int. Cl. C12Q 1/689

(52) U.S. Cl. CPC C12Q 1/689; C12Q 2600/106; C12Q 2600/16; C12Q 2600/136; C12Q 2600/156; C12Q 2600/112

(57) ABSTRACT

The present invention relates to a method for determining in a bacterial sample information on both the phylogenetic origin and the antibiotic resistances of bacterial strains. The present invention further relates a kit for performing the method of the present invention.

The present invention relates to a molecular method for determining in a bacterial sample both the phylogenetic background and the antibiotic resistances of bacterial strains and to a kit for performing the method.

The responsibility of clinical microbiology laboratories is (i) to identify the bacterial strain, (ii) to perform antibiotic susceptibility testing (AST) and (iii) to provide a surveillance infrastructure. The diagnostic laboratories report to clinicians in the hospitals or to practitioners in order to guide them in their treatment choice or the implementation of hygiene measures.

Today AST involves phenotypic test methods which mostly rely on semi-automated systems. Despite the clinical need, microbiological diagnostics of resistance has experienced little fundamental change over the years. Bacterial antibiotic susceptibility testing still predominantly relies on culture-dependent methods. As a consequence, clinical microbiology is still laborintensive and slow in comparison to other fields of laboratory medicine. Efforts to decrease the turn-round time of culture for susceptibility testing have been made. Those include the combination of powerful optical systems for growth detection in miniaturized cups and the computerized analysis of growth patterns. Nevertheless, the gain in terms of turn-round time of these new culture-dependent methods remains limited. There are only few molecular resistance profiling assays on the market which provide rapid results in order to guide the clinicians in their treatment choice. However those assays can be applied only on a small selection of pathogens, they do not cover the entire “Resistome” of the pathogens and are far too expensive to be introduced into routine diagnostic microbiology. The high price precludes also their broad application for surveillance purposes.

To significantly decrease hospital acquired infections and to prevent outbreaks, it is essential to establish surveillance systems. Management of nosocomial infections, in particular those caused by multidrug-resistant gram negative bacteria, requires a strategy that includes not only the development of new anti-microbial compounds and a rational use of anti-microbial agents, but also early diagnosis for targeted treatment and the implementation of effective infection control principles. In order to do so, in addition to species identification typing of the causing bacterial isolates is essential. This is currently mainly performed by the use of pulse field gel electrophoresis or PCR-based technologies such as e.g. multilocus sequence typing (MLST) assays.

There have been no attempts to use genetic markers of resistance conferring genes for the development of a molecular typing system and to combine resistance typing and genotyping within one molecular method.

In spite of the initial enthusiasm and the huge literature on their diagnostic use, gene-detection-based molecular methods have not yet had the dramatic impact on routine diagnostic microbiology that many predicted. However, the identification of antibiotic susceptibility based on resistance determining molecular markers in combination with the identification of phylogenetic origin may provide the basis for therapeutic advice regarding antibiotic treatment. At the same time, it would provide information on the epidemiologic behavior of this specific isolate. Since cost, morbidity and mortality are significantly lower when phylogenetic information and antibiotic resistances are determined more rapidly, such method would have a great impact on infection management.

In order to meet these challenges the present inventors have established a rapid, cost-effective and robust molecular method. The method is a gene-detection-based high-density molecular method which tests for a plethora of genetic determinants to provide timely information on resistance profiles as well as on phylogenetic origin.

In comparison to other gene-detection-based molecular methods, the method of the present invention has several advantages. As compared to the previously developed molecular methods of resistance testing, the method of the present invention can be applied very broadly to any gram-negative bacterial pathogen as it is not restricted to the detection of specific and speciesrelated genetic determinants. Instead, the method of the present invention comprises a multitude of genetic markers that can be found in various combinations in gram-negative multidrug-resistant bacterial pathogens of diverse species. Furthermore, the method of the present invention may be applied on the MassARRAY (Agena Bioscience) which involves a multiplex PCR coupled with mass spectrometry technology. This technology is robust and rapid and most importantly allows for the detection of many markers at reasonable costs.

Determination of antibiotic resistance by detection of single nucleotide polymorphisms (SNPs) is known from the art. For example U.S. Pat. No. 8,247,170 B2 describes detection of penicillin tolerance in Group B Streptococcus via SNPs in the gene encoding penicillin binding protein 4. U.S. Pat. No. 8,741,563 B2 describes correlation between metronidazole resistance in Trichomonas vaginalis and SNPs in tvntr 4 and tvntr 6 genes. US 2012/0009572 A1 describes detection of Methicillin resistance in Staphylococcus aureus via SNPs in the gene encoding penicillin binding protein 3. However, all these attempts are limited to detection of resistance of known bacterial species against specific antibiotic drugs. Furthermore, none of these methods provides information on the phylogenetic origin of the bacterial strain.

Syrmis et al. (“Comparison of a multiplexed MassARRAY system with real-time allele-specific PCR technology for genotyping of methicillin-resistant Staphylococcus aureus”; Clinical Microbiology and Infection, Volume 17, Number 12, Pages 1804-1810, December 2011) discloses a method for genotyping of methicillin-resistant Staphylococcus aureus. However, only classical phylogenetic markers of S. aureus are used for phylogenetic analysis. Obtaining phylogenetic information based on antibiotic resistance conferring genes is not disclosed. Hence, the method of Syrmis et al. cannot be applied for analysis of different bacterial species because the sequences of the described phylogenetic markers are very specific for S. aureus.

There is a plethora of resistance conferring genes which are acquired in different combinations by different gram-negative multi-drug resistant isolates. Furthermore those resistance conferring genes exhibit substantial variation in their genomic sequence. The inventors found that if a gene-detection-based high-density molecular system provides information on the presence/absence and the sequence variation of those resistances conferring genes this opens up the unique opportunity to use resistance determinants not only for resistance typing but also for obtaining information on the phylogenetic origin. Moreover, they found that if this resistance determinant phylogenetic information is combined with only a restricted selection of phylogenetic markers, the identification of phylogenetic origin can be even further improved. Information on phylogenetic origin can be used for generating phylogenetic trees and preferably enables subclassification of the bacterial species. This allows for the detection of transmissions e.g. from patient to patient.

It was therefore an object of the present invention to provide a method that overcomes the problems associated with the prior art methods. The object of the present invention was in particular to derive a high amount of information from a method of low effort. The problem is solved by the subject-matter of the patent claims.

The problem is in particular solved by a method for determining from a bacterial sample information on both the phylogenetic origin and the antibiotic resistances of bacterial strains, the method comprising the steps of:
A) determining which nucleotide is present
   a. at position 814 in the KPC-2 coding sequence (SEQ ID NO:87),
   b. at position 82 in the NDM-1 coding sequence (SEQ ID NO:88),
   c. at position 104 in the OXA-9 coding sequence (SEQ ID NO:89),
   d. at position 186 in the OXA-48 coding sequence (SEQ ID NO:90),
   e. at position 556 in the CTX-M-9 coding sequence (SEQ ID NO:91),
   f. at position 453 in the CTX-M-15 coding sequence (SEQ ID NO:92),
   g. at position 223 in the AAC(6)-lb-cr coding sequence (SEQ ID NO:93), and
   h. at position 454 in the AAC(6)-lb-cr coding sequence (SEQ ID NO:93),
or at analogous positions in variants or homologous coding sequences having at least 80% sequence identity with the indicated coding sequences, and
B) determining information on both the phylogenetic origin and antibiotic resistances of the bacterial strain based on a comparison of the pattern of the determined nucleotides with respective predetermined patterns of bacterial strain, which phylogenetic origin and antibiotic resistances are known.

Step A) described above may optionally further comprise determining which nucleotide is present
(I) at position 408 in the OXA-1 coding sequence (SEQ ID NO:163),
(II) at position 585 in the OXA-2 coding sequence (SEQ ID NO:164),
(III) at position 228 in the TEM-1 coding sequence (SEQ ID NO:165),
(IV) at position 230 in the APHA3-la coding sequence (SEQ ID NO:166), and/or
(V) at position 63 in the AAC(3′)-IIae coding sequence (SEQ ID NO:167),
   or at analogous positions in variants or homologous coding sequences having at least 80% sequence identity with the indicated coding sequences.

In particularly preferred embodiments of the present invention, at least one, more preferably at least two, more preferably at least three, more preferably at least four, more preferably all five of the nucleotides being present at the indicated positions (I) to (V) are determined.

Sequence information that can be used for the purpose of comparison is known for a plethora of isolates of many bacterial species and is available in from databases well known to the person skilled in the art.

Preferably, the variants or homologous coding sequences have a sequence identity with the coding sequences as depicted in SEQ ID NOs: 87-93 of at least 90%, more preferably of at least 95%, more preferably of at least 98%, even more preferably of at least 99%.

Preferably, the variants or homologous coding sequences have a sequence identity with the coding sequences as depicted in SEQ ID NOs: 163-167 of at least 90%, more preferably of at least 95%, more preferably of at least 98%, even more preferably of at least 99%.

The term “% sequence identity” is well known in the art and is very familiar to the skilled person. Briefly, when a nucleotide sequence is compared by alignment to a sequence as given in the sequence listing of the present invention, the number of nucleotides that are identical in both sequences can be easily identified. The term “% sequence identity” describes the ratio of this number of identical nucleotides to the total number of nucleotide residues of the nucleotide sequence of the present invention as given in the sequence listing. In other words, using an alignment, for two sequences the percentage of nucleotide residues that are the same (e.g., 90% or 95% identity) may be determined, when the sequences are compared and aligned for maximum correspondence, for example by using a sequence comparison algorithm as known in the art.

It is preferable that the bacterial strains are multi-drug resistant. In particular, it is advantageous if the bacterial strain contains at least 3, more preferably at least 4, more preferably at least 5, more preferably at least 6 and even more preferably all 7 coding sequences of SEQ ID NOs: 87-93 indicated above. It is also advantageous if the bacterial strain contains at least 1, more preferably at least 2, more preferably at least 3, more preferably at least 4 and even more preferably all 5 coding sequences of SEQ ID NOs: 163-167 indicated above.

The SNPs described above are universally distributed among multi-drug resistant isolates of many different bacterial species. Therefore, the method of the invention can be excellently applied to all multi-drug resistant bacterial isolates.

The bacterial sample that is analyzed with the method of the present invention may be any sample that contains bacterial DNA in an amount sufficient for subsequent analysis. Preferably, the sample is derived from the human or animal body. The sample may for example be a blood sample, a urine sample, a fecal sample, a saliva sample, a sample from a smear test of skin or mucosa or a sample of wound secretion. Preferably, the sample can be easily derived from the human or animal body without greater efforts. In embodiments of the present invention, the above described samples may be further processed by isolation of bacteria and/or bacterial DNA in order to obtain the bacterial sample to be analyzed by the method of the present invention.

The present inventors found that the pattern of single nucleotide polymorphisms (SNPs) at the indicated positions in resistance conferring genes enables determination of information on both the phylogenetic origin and antibiotic resistances of bacterial strains in a surprisingly sensitive manner. Thus, the respective SNP pattern forms a “molecular fingerprint” that is extremely useful for early diagnosis of infectious diseases. So far, identification of phylogenetic origin of bacterial isolates was rather focused on SNPs in housekeeping genes. However, the present inventors found that opportunistic bacterial pathogens have acquired distinct sets of resistance conferring genes, a part of which surprisingly exhibits substantial variation to such an extent that obtaining information on phylogenetic origin is enabled by determining the specific SNP pattern of selected positions in those genes. Thus, the pattern of the nucleotides described under step A) above was found to have sufficient discriminative power for distinguishing clonal lineages within species. In addition to selection of suitable therapy, determination of the clonal lineage may also contribute to tracking the course of infection spreading, which may help to optimize infection management and improve hygienic standards.

As such SNPs are universally distributed among multi-drug resistant isolates of many different bacterial species, the method can be excellently applied to all multi-drug resistant bacterial isolates. It is preferable that the bacterial strains are multi-drug resistant. Furthermore, it is preferable, that the bacterial strain is a gram-negative bacterium. In particularly preferred embodiments, the bacterial strain is a multi-drug resistant gram-negative bacterium. More preferably, the multi-drug resistant bacterial strain is selected from the family of Enterobacteriaceae or from the genus of Pseudomonas or Acinetobacter. More preferably, the genus of the multi-drug resistant bacterial strain is selected from the group consisting of Klebsiella, Escherichia, Pseudomonas and Acinetobacter. More preferably, the multi-drug resistant bacterial strain belongs to the genus selected from the group consisting of Klebsiella and Escherichia. More preferably, the multi-drug resistant bacterial strain belongs to the genus Klebsiella. Even more preferably, the multi-drug resistant bacterial strain is a Klebsiella pneumoniae or Escherichia coli strain. Even more preferably, the multi-drug resistant bacterial strain is a Klebsiella pneumoniae strain. In other preferred embodiments, the multi-drug resistant bacterial strain is an Escherichia coli strain. In alternative embodiments, the bacterial strain is a gram-positive bacterium.

All genes listed under step A) above are related to antibiotic resistance in bacteria. In particular, KPC-2, NDM-1, OXA-9 and OXA-48 are carbapenemases, CTX-M-9 and CTX-M-15 are betalactamases and AAC(6′)-lb-cr is an aminoglycoside-modifying enzyme. OXA-1 and OXA-2 also belong to the class of carbapenemases, TEM-1 is a beta-lactamase, more specifically a type I penicillinase and APHA3-la and AAC(3′)IIae are aminoglycoside-modifying enzymes. The SNPs and mutations utilized according to step A) described above are summarized in the following table 1. The nucleotide position is the position of the respective nucleotide in the indicated coding sequences of SEQ ID NOs: 87-93.

TABLE 1

| | Nucleotide | Most common | |
|---|---|---|---|
| Coding Sequence | position | nucleotide | Information |
| KPC-2 (SEQ ID NO: 87) | 814 | C or T | C indicates KPC-2 group; |
| | | | T indicates KPC-3 group |
| NDM-1 (SEQ ID NO: 88) | 82 | C or G | C indicates NDM-1 and |
| | | | others; G indicates NDM-2 |
| OXA-9 (SEQ ID NO: 89) | 104 | T | No SNP at this position; |
| | | | rather detection of T indicates |
| | | | presence of OXA-9 |
| | | | gene |
| OXA-48 (SEQ ID NO: 90) | 186 | G or A | G indicates OXA-48 and |
| | | | others; A indicates OXA- |
| | | | 54 |
| CTX-M-9 (SEQ ID NO: 91) | 556 | C or G | C indicates CTX-M-9; G |
| | | | indicates CTX-M-45 |
| CTX-M-15 (SEQ ID NO: 92) | 453 | C or T | C indicates CTX-M-15 and |
| | | | others; T indicates CTX-M- |
| | | | 10/34/53 |
| AAC(6′)-lb-cr (SEQ ID NO: 93) | 223 | A or C or T | A or C indicates AAC(6′)- |
| | | | lb-cr; T indicates AAC(6′)- |
| | | | lb |
| AAC(6′)-lb-cr (SEQ ID NO: 93) | 454 | T or G | T indicates AAC(6′)-lb-cr; |
| | | | G indicates AAC(6′)-lb |

The SNPs and mutations optionally utilized according to the SNP positions (I) to (V) described above are summarized in the following table 2. The nucleotide position is the position of the respective nucleotide in the indicated coding sequences of SEQ ID NOs: 163-167.

TABLE 2

| | Nucleotide | Most common |
|---|---|---|
| Coding Sequence | position | nucleotide |
| OXA-1 (SEQ ID NO: 163) | 408 | G or A |
| OXA-2 (SEQ ID NO: 164) | 585 | T or C |
| TEM-1 (SEQ ID NO: 165) | 228 | T or C |
| APHA3-Ia (SEQ ID NO: 166) | 230 | A or C |
| AAC(3′)-IIae (SEQ ID NO: 167) | 63 | G or C or A |

Preferably, in order to determine the nucleotides according to step A) of the method, DNA is isolated from the bacterial sample in an initial step. Isolation of DNA from the sample may be performed by any suitable DNA isolation method. Such methods are well known to the skilled person and typically include cell lysis followed by DNA isolation either by DNA precipitation, by phenol-chloroform extraction or by adsorption of DNA to specific DNA binding matrices as for example a silica gel.

Preferably, bacterial DNA comprising the nucleotides of interest is amplified prior to determination of the nucleotides. Preferably, DNA amplification is done by polymerase chain reaction (PCR). Preferably, the PCR is a multiplex PCR, thus enabling amplification of several different DNA sequences simultaneously. Multiplex PCRs are advantageous because targeting multiple sequences at once enables gaining information from a single test run that otherwise would require several times the reagents and more time to perform. However, successfully setting-up a multiplex PCR is not trivial because it requires all different primer pairs to work at the same annealing temperature during PCR. Furthermore, the increased number of primers being present in a multiplex PCR reaction as compared to a singleplex PCR is associated with an increased probability of undesired interactions of primers which in turn may compromise the sensitivity and/or specificity of the multiplex PCR. Therefore, primer sets for multiplex PCRs are difficult to develop.

The present inventors developed primer sets that are useful for amplification of relevant regions of bacterial DNA with high specificity and sensitivity, particularly in multiplex PCR reactions. Sequences that are preferably comprised by these PCR primers are presented in the sequence listing of the present invention and are summarized in the following table 3. The sequences indicated in table 3 are most preferred. However, variants having a sequence identity of at least 80%, more preferably at least 85% more preferably at least 90%, more preferably at least 94% with the sequences indicated in table 3 are also preferable sequences of the present invention.

TABLE 3

| | SEQ | |
|---|---|---|
| | ID | Comprised |
| Primer name | NO: | sequence (5′-3′) |
| KPC-2 forward | 46 | CTATTGTGTTGGCCGTCTAC |
| KPC-2 reverse | 47 | AATCCCTCGAGCGCGAGTCT |
| NDM-1 forward | 48 | CCAATATTATGCACCCGGTC |
| NDM-1 reverse | 49 | GAGCTGGCGGAAAACCAGAT |
| OXA-9 forward | 51 | ATGTTGGTGTTCGTTTCCGC |
| OXA-9 reverse | 52 | GTTTAAAAGACGAGCACGGA |
| OXA-48 forward | 54 | GAGAATAAGCAGCAAGGAT |
| OXA-48 reverse | 55 | CCATCCCACTTAAAGACTTG |
| CTX-M-9 forward | 57 | GTTTCGTCTGGATCGCACTG |
| CTX-M-9 reverse | 58 | TGGGTTTCGCCCAGCGCAT |
| CTX-M-15 forward | 60 | GCGCTACAGTACAGCGATAA |
| CTX-M-15 reverse | 61 | AGACGGAACGTTTCGTCTC |
| AAC(6′)-Ib-cr (223) | 62 | AATGCTGAATGGAGAGCCGA |
| forward | | |
| AAC(6′)-Ib-cr (223) | 63 | TTGAACAGCAACTCAACCAG |
| reverse | | |
| AAC(6′)-Ib-cr (454) | 65 | GATCCGATGCTACGAGAAAG |
| forward | | |
| AAC(6′)-Ib-cr (454) | 66 | GTTTGAACCATGTACACGGC |
| reverse | | |
| OXA-1 forward | 134 | TGTTGTTTGGGTTTCGCAAG |
| OXA-1 reverse | 135 | CCAGAGAAGTCTTGATTTCC |
| OXA-2 forward | 136 | GCATTTCTCAGGAAGCTCTA |
| OXA-2 reverse | 137 | CCTTCCCAGCCCGTCTTTG |
| TEM-1 forward | 138 | GATCCTTGAGAGTTTTCGCC |
| TEM-1 reverse | 139 | GCTTTTCTGTGACTGGTGAG |
| AP HA3-Ia forward | 141 | AGGTAGCGTTGCCAATGATG |
| AP HA3-Ia reverse | 142 | TGCTTGATAGTCGGAAGAGG |
| AAC(3′)-IIae forward | 144 | ATACGCGGAAGGCAATAACG |
| AAC(3′)-IIae reverse | 145 | CCAATCGATTTAAGTGAGGC |

By using a combination of corresponding forward and reverse primers, DNA fragments containing the SNP positions of the present invention can be excellently amplified, in particular as PCR products.

In embodiments of the present invention, the amplifying primers do not only comprise the sequences indicated in table 3 or variants having a sequence identity of at least 80%, more preferably at least 85% more preferably at least 90%, more preferably at least 94% therewith but even consist of these sequences. However, the present inventors found that the amplification is advantageously balanced if the amplification primers additionally comprise nucleotides at the 5′-end. Furthermore, this increases the masses of unused primers (out of the mass range on the spectra). Preferably, in addition to the sequences described in table 3 or to variants thereof as indicated above, the amplification primers comprise at least 5, more preferably at least 8, more preferably at least 9, more preferably at least 10 and even more preferably exactly 10 additional nucleotides at the 5′-end. The number of additional nucleotides at the 5′-end should be limited and therefore the amplification primers preferably comprise at most 20, more preferably at most 15, more preferably at most 12, more preferably at most 11 and even more preferably at most 10 additional nucleotides at the 5′-end. Sequences that are comprised in most preferred amplification primers of the present invention are summarized in the following table 4. However, variants having a sequence identity of at least 80%, more preferably at least 85% more preferably at least 90%, more preferably at least 95% with the sequences indicated in table 4 are also preferable sequences of the present invention.

TABLE 4

| Primer name | SEQ ID NO: | Comprised sequence (5′-3′) |
|---|---|---|
| KPC-2 long forward | 1 | ACGTTGGATGCTATTGTGTTGGCCGTCTAC |
| KPC-2 long reverse | 2 | ACGTTGGATGAATCCCTCGAGCGCGAGTCT |
| NDM-1 long forward | 4 | ACGTTGGATGCCAATATTATGCACCCGGTC |
| NDM-1 long reverse | 5 | ACGTTGGATGGAGCTGGCGGAAAACCAGAT |
| OXA-9 long forward | 7 | ACGTTGGATGATGTTGGTGTTCGTTTCCGC |
| OXA-9 long reverse | 8 | ACGTTGGATGGTTTAAAAGACGAGCACGGA |
| OXA-48 long forward | 10 | ACGTTGGATGGAGAATAAGCAGCAAGGAT |
| OXA-48 long reverse | 11 | ACGTTGGATGCCATCCCACTTAAAGACTTG |
| CTX-M-9 long forward | 13 | ACGTTGGATGGTTTCGTCTGGATCGCACTG |
| CTX-M-9 long reverse | 14 | ACGTTGGATGTGGGTTTCGCCCAGCGCAT |
| CTX-M-15 long | 16 | ACGTTGGATGGCGCTACAGTACAGCGATAA |
| forward | | |
| CTX-M-15 long | 17 | ACGTTGGATGAGACGGAACGTTTCGTCTC |
| reverse | | |
| AAC(6′)-Ib-cr (223) long for- | 19 | ACGTTGGATGAATGCTGAATGGAGAGCCGA |
| ward | | |
| AAC(6′)-Ib-cr (223) long re- | 20 | ACGTTGGATGTTGAACAGCAACTCAACCAG |
| verse | | |
| AAC(6′)-Ib-cr (454) long for- | 22 | ACGTTGGATGGATCCGATGCTACGAGAAAG |
| ward | | |
| AAC(6′)-Ib-cr (454) long re- | 23 | ACGTTGGATGGTTTGAACCATGTACACGGC |
| verse | | |
| OXA-1 long forward | 95 | ACGTTGGATGTGTTGTTTGGGTTTCGCAAG |
| OXA-1 long reverse | 96 | ACGTTGGATGCCAGAGAAGTCTTGATTTCC |
| OXA-2 long forward | 98 | ACGTTGGATGGCATTTCTCAGGAAGCTCTA |
| OXA-2 long reverse | 99 | ACGTTGGATGCCTTCCCAGCCCGTCTTTG |
| TEM-1 long forward | 101 | ACGTTGGATGGATCCTTGAGAGTTTTCGCC |
| TEM-1 long reverse | 102 | ACGTTGGATGGCTTTTCTGTGACTGGTGAG |
| APHA3-Ia long forward | 104 | ACGTTGGATGAGGTAGCGTTGCCAATGATG |
| APHA3-Ia long reverse | 105 | ACGTTGGATGTGCTTGATAGTCGGAAGAGG |
| AAC(3′)-IIae long forward | 107 | ACGTTGGATGATACGCGGAAGGCAATAACG |
| AAC(3′)-IIae long reverse | 108 | ACGTTGGATGCCAATCGATTTAAGTGAGGC |

Preferably, the amplifying primers do not only comprise the sequences indicated in table 4 or variants having a sequence identity of at least 80%, more preferably at least 85% more preferably at least 90%, more preferably at least 95% therewith but even consist of these sequences.

The problem of the present invention is also solved by a method for determining from a bacterial sample information on both the phylogenetic origin and the antibiotic resistances of bacterial strains, the method comprising the step of:
A) performing PCR reactions comprising the following primer pairs:
   a. Primer pair 1 consisting of a forward primer comprising a sequence of SEQ ID NO:46 or a sequence having at least 80%, preferably at least 85%, more preferably at least 90%, more preferably at least 94% sequence identity therewith, and a reverse primer comprising a sequence of SEQ ID NO:47 or a sequence having at least 80%, preferably at least 85%, more preferably at least 90%, more preferably at least 94% sequence identity therewith,
   b. Primer pair 2 consisting of a forward primer comprising a sequence of SEQ ID NO:48 or a sequence having at least 80%, preferably at least 85%, more preferably at least 90%, more preferably at least 94% sequence identity therewith, and a reverse primer comprising a sequence of SEQ ID NO:49 or a sequence having at least 80%, preferably at least 85%, more preferably at least 90%, more preferably at least 94% sequence identity therewith,
   c. Primer pair 3 consisting of a forward primer comprising a sequence of SEQ ID NO:51 or a sequence having at least 80%, preferably at least 85%, more preferably at least 90%, more preferably at least 94% sequence identity therewith, and a reverse primer comprising a sequence of SEQ ID NO:52 or a sequence having at least 80%, preferably at least 85%, more preferably at least 90%, more preferably at least 94% sequence identity therewith,
   d. Primer pair 4 consisting of a forward primer comprising a sequence of SEQ ID NO:54 or a sequence having at least 80%, preferably at least 85%, more preferably at least 90%, more preferably at least 94% sequence identity therewith, and a reverse primer comprising a sequence of SEQ ID NO:55 or a sequence having at least 80%, preferably at least 85%, more preferably at least 90%, more preferably at least 94% sequence identity therewith,
   e. Primer pair 5 consisting of a forward primer comprising a sequence of SEQ ID NO:57 or a sequence having at least 80%, preferably at least 85%, more preferably at least 90%, more preferably at least 94% sequence identity therewith, and a reverse primer comprising a sequence of SEQ ID NO:58 or a sequence having at least 80%, preferably at least 85%, more preferably at least 90%, more preferably at least 94% sequence identity therewith,
   f. Primer pair 6 consisting of a forward primer comprising a sequence of SEQ ID NO:60 or a sequence having at least 80%, preferably at least 85%, more preferably at least 90%, more preferably at least 94% sequence identity therewith, and a reverse primer comprising a sequence of SEQ ID NO:61 or a sequence having at least 80%, preferably at least 85%, more preferably at least 90%, more preferably at least 94% sequence identity therewith,
   g. Primer pair 7 consisting of a forward primer comprising a sequence of SEQ ID NO:62 or a sequence having at least 80%, preferably at least 85%, more preferably at least 90%, more preferably at least 94% sequence identity therewith, and a reverse primer comprising a sequence of SEQ ID NO:63 or a sequence having at least 80%, preferably at least 85%, more preferably at least 90%, more preferably at least 94% sequence identity therewith, and
   h. Primer pair 8 consisting of a forward primer comprising a sequence of SEQ ID NO:65 or a sequence having at least 80%, preferably at least 85%, more preferably at least 90%, more preferably at least 94% sequence identity therewith, and a reverse primer comprising a sequence of SEQ ID NO:66 or a sequence having at least 80%, preferably at least 85%, more preferably at least 90%, more preferably at least 94% sequence identity therewith.

Step A) described above may optionally further comprise performing PCR reactions comprising one or more of the following primer pairs:
(I) Primer pair 9 consisting of a forward primer comprising a sequence of SEQ ID NO:134 or a sequence having at least 80%, preferably at least 85%, more preferably at least 90%, more preferably at least 94% sequence identity therewith, and a reverse primer comprising a sequence of SEQ ID NO:135 or a sequence having at least 80%, preferably at least 85%, more preferably at least 90%, more preferably at least 94% sequence identity therewith,
(II) Primer pair 10 consisting of a forward primer comprising a sequence of SEQ ID NO:136 or a sequence having at least 80%, preferably at least 85%, more preferably at least 90%, more preferably at least 94% sequence identity therewith, and a reverse primer comprising a sequence of SEQ ID NO:137 or a sequence having at least 80%, preferably at least 85%, more preferably at least 90%, more preferably at least 94% sequence identity therewith,
(III) Primer pair 11 consisting of a forward primer comprising a sequence of SEQ ID NO:138 or a sequence having at least 80%, preferably at least 85%, more preferably at least 90%, more preferably at least 94% sequence identity therewith, and a reverse primer comprising a sequence of SEQ ID NO:139 or a sequence having at least 80%, preferably at least 85%, more preferably at least 90%, more preferably at least 94% sequence identity therewith,
(IV) Primer pair 12 consisting of a forward primer comprising a sequence of SEQ ID NO:141 or a sequence having at least 80%, preferably at least 85%, more preferably at least 90%, more preferably at least 94% sequence identity therewith, and a reverse primer comprising a sequence of SEQ ID NO:142 or a sequence having at least 80%, preferably at least 85%, more preferably at least 90%, more preferably at least 94% sequence identity therewith, and/or
(V) Primer pair 13 consisting of a forward primer comprising a sequence of SEQ ID NO:144 or a sequence having at least 80%, preferably at least 85%, more preferably at least 90%, more preferably at least 94% sequence identity therewith, and a reverse primer comprising a sequence of SEQ ID NO:145 or a sequence having at least 80%, preferably at least 85%, more preferably at least 90%, more preferably at least 94% sequence identity therewith.

In particularly preferred embodiments of the present invention, PCR reactions are performed comprising at least one, more preferably at least two, more preferably at least three, more preferably at least four, more preferably all five primer pairs indicated under points (I) to (V) above.

All primers of the primer pairs indicated under points a. to h. and (I) to (V) above may comprise a sequence having at least 80%, more preferably at least 85%, more preferably at least 90%, more preferably at least 94% sequence identity with the sequences of the indicated SEQ ID NOs.

According to the present invention, the PCR reaction of each primer pair may be performed in a separate tube. Thus, 8 separate PCR reactions may be performed for the primer pairs indicated under points a. to h. above. The 5 optional PCR reactions comprising the primer pairs indicated under points (I) to (V) above may be performed as separate PCR reactions as well. However, it is preferable that the number of separate PCR reactions is reduced in order to save time and resources. Preferably, all PCR reactions, in particular those comprising the primers pairs indicated under points a. to h. above and optionally one or more of the primer pairs indicated under points (I) to (V) above, are performed in a single tube.

In embodiments of the present invention, the forward and reverse primers do not only comprise the sequences of SEQ ID NOs: 46-49, 51, 52, 54, 55, 57, 58, 60-63, 65, 66, 134-139, 141, 142, 144, 145 as indicated above or variants having a sequence identity of at least 80%, more preferably at least 85% more preferably at least 90%, more preferably at least 94% therewith but even consist of these sequences. However, the present inventors found that the amplification is advantageously balanced if the forward and reverse primers additionally comprise nucleotides at the 5′-end. Furthermore, this increases the masses of unused primers (out of the mass range on the spectra).

Therefore, a particular preferred method of the present invention for determining from a bacterial sample information on both the phylogenetic origin and the antibiotic resistances of bacterial strains comprises the step of:
A) performing PCR reactions comprising the following primer pairs:
   a. Primer pair 1 consisting of a forward primer comprising a sequence of SEQ ID NO:1 or a sequence having at least 80%, preferably at least 85%, more preferably at least 90%, more preferably at least 95% sequence identity therewith, and a reverse primer comprising a sequence of SEQ ID NO:2 or a sequence having at least 80%, preferably at least 85%, more preferably at least 90%, more preferably at least 95% sequence identity therewith,
   b. Primer pair 2 consisting of a forward primer comprising a sequence of SEQ ID NO:4 or a sequence having at least 80%, preferably at least 85%, more preferably at least 90%, more preferably at least 95% sequence identity therewith, and a reverse primer comprising a sequence of SEQ ID NO:5 or a sequence having at least 80%, preferably at least 85%, more preferably at least 90%, more preferably at least 95% sequence identity therewith,
   c. Primer pair 3 consisting of a forward primer comprising a sequence of SEQ ID NO:7 or a sequence having at least 80%, preferably at least 85%, more preferably at least 90%, more preferably at least 95% sequence identity therewith, and a reverse primer comprising a sequence of SEQ ID NO:8 or a sequence having at least 80%, preferably at least 85%, more preferably at least 90%, more preferably at least 95% sequence identity therewith,
   d. Primer pair 4 consisting of a forward primer comprising a sequence of SEQ ID NO:10 or a sequence having at least 80%, preferably at least 85%, more preferably at least 90%, more preferably at least 95% sequence identity therewith, and a reverse primer comprising a sequence of SEQ ID NO:11 or a sequence having at least 80%, preferably at least 85%, more preferably at least 90%, more preferably at least 95% sequence identity therewith,
   e. Primer pair 5 consisting of a forward primer comprising a sequence of SEQ ID NO:13 or a sequence having at least 80%, preferably at least 85%, more preferably at least 90%, more preferably at least 95% sequence identity therewith, and a reverse primer comprising a sequence of SEQ ID NO:14 or a sequence having at least 80%, preferably at least 85%, more preferably at least 90%, more preferably at least 95% sequence identity therewith,
   f. Primer pair 6 consisting of a forward primer comprising a sequence of SEQ ID NO:16 or a sequence having at least 80%, preferably at least 85%, more preferably at least 90%, more preferably at least 95% sequence identity therewith, and a reverse primer comprising a sequence of SEQ ID NO:17 or a sequence having at least 80%, preferably at least 85%, more preferably at least 90%, more preferably at least 95% sequence identity therewith,
   g. Primer pair 7 consisting of a forward primer comprising a sequence of SEQ ID NO:19 or a sequence having at least 80%, preferably at least 85%, more preferably at least 90%, more preferably at least 95% sequence identity therewith, and a reverse primer comprising a sequence of SEQ ID NO:20 or a sequence having at least 80%, preferably at least 85%, more preferably at least 90%, more preferably at least 95% sequence identity therewith, and
   h. Primer pair 8 consisting of a forward primer comprising a sequence of SEQ ID NO:22 or a sequence having at least 80%, preferably at least 85%, more preferably at least 90%, more preferably at least 95% sequence identity therewith, and a reverse primer comprising a sequence of SEQ ID NO:23 or a sequence having at least 80%, preferably at least 85%, more preferably at least 90%, more preferably at least 95% sequence identity therewith.

Step A) described above may optionally further comprise performing PCR reactions comprising one or more of the following primer pairs:
(I) Primer pair 9 consisting of a forward primer comprising a sequence of SEQ ID NO:95 or a sequence having at least 80%, preferably at least 85%, more preferably at least 90%, more preferably at least 95% sequence identity therewith, and a reverse primer comprising a sequence of SEQ ID NO:96 or a sequence having at least 80%, preferably at least 85%, more preferably at least 90%, more preferably at least 95% sequence identity therewith,
(II) Primer pair 10 consisting of a forward primer comprising a sequence of SEQ ID NO:98 or a sequence having at least 80%, preferably at least 85%, more preferably at least 90%, more preferably at least 95% sequence identity therewith, and a reverse primer comprising a sequence of SEQ ID NO:99 or a sequence having at least 80%, preferably at least 85%, more preferably at least 90%, more preferably at least 95% sequence identity therewith,
(III) Primer pair 11 consisting of a forward primer comprising a sequence of SEQ ID NO:101 or a sequence having at least 80%, preferably at least 85%, more preferably at least 90%, more preferably at least 95% sequence identity therewith, and a reverse primer comprising a sequence of SEQ ID NO:102 or a sequence having at least 80%, preferably at least 85%, more preferably at least 90%, more preferably at least 95% sequence identity therewith,
(IV) Primer pair 12 consisting of a forward primer comprising a sequence of SEQ ID NO:104 or a sequence having at least 80%, preferably at least 85%, more preferably at least 90%, more preferably at least 95% sequence identity therewith, and a reverse primer comprising a sequence of SEQ ID NO:105 or a sequence having at least 80%, preferably at least 85%, more preferably at least 90%, more preferably at least 95% sequence identity therewith, and/or
(V) Primer pair 13 consisting of a forward primer comprising a sequence of SEQ ID NO:107 or a sequence having at least 80%, preferably at least 85%, more preferably at least 90%, more preferably at least 95% sequence identity therewith, and a reverse primer comprising a sequence of SEQ ID NO:108 or a sequence having at least 80%, preferably at least 85%, more preferably at least 90%, more preferably at least 95% sequence identity therewith.

In particularly preferred embodiments of the present invention, PCR reactions are performed comprising at least one, more preferably at least two, more preferably at least three, more preferably at least four, more preferably all five primer pairs indicated under points (I) to (V) above.

All primers of the primer pairs indicated under points a. to h. and (I) to (V) above may comprise a sequence having at least 80%, more preferably at least 85%, more preferably at least 90%, more preferably at least 95% sequence identity with the sequences of the indicated SEQ ID NOs.

According to the present invention, the PCR reaction of each primer pair may be performed in a separate tube. Thus, 8 separate PCR reactions may be performed for the primer pairs indicated under points a. to h. above. The 5 optional PCR reactions comprising the primer pairs indicated under points (I) to (V) above may be performed as separate PCR reactions as well. However, it is preferable that the number of separate PCR reactions is reduced in order to save time and resources. Preferably, all PCR reactions, in particular those comprising the primers pairs indicated under points a. to h. above and optionally one or more of the primer pairs indicated under points (I) to (V) above, are performed in a single tube.

In preferred embodiments of the present invention, the forward and reverse primers do not only comprise the sequences of SEQ ID NOs: 1, 2, 4, 5, 7, 8, 10, 11, 13, 14, 16, 17, 19, 20, 22, 23, 95, 96, 98, 99, 101, 102, 104, 105, 107, 108 as indicated above or variants having a sequence identity of at least 80%, more preferably at least 85% more preferably at least 90%, more preferably at least 95% therewith but even consist of these sequences.

In the methods described above, the step of performing PCR reactions comprising primer pairs 1 to 8 preferably results in PCR products, in particular in amplification of regions of interest comprised in the KPC-2 coding sequence (SEQ ID NO:87), the NDM-1 coding sequence (SEQ ID NO:88), the OXA-9 coding sequence (SEQ ID NO:89), the OXA-48 coding sequence (SEQ ID NO:90), the CTX-M-9 coding sequence (SEQ ID NO:91), the CTX-M-15 coding sequence (SEQ ID NO:92), and the AAC(6′)-lb-cr coding sequence (SEQ ID NO:93), or in variants or homologous coding sequences having at least 80%, more preferably at least 90%, more preferably at least 95%, more preferably at least 98%, even more preferably at least 99% sequence identity with the indicated coding sequences in case the respective coding sequences are present in the analyzed sample. Exemplary binding regions of the amplification primers and of the SNPs of interest are shown in FIG. 1.

Particularly preferably, primer pair 1 results in amplification of a region comprising the nucleotide at position 814 of the KPC-2 coding sequence (SEQ ID NO:87), primer pair 2 results in amplification of a region comprising the nucleotide at position 82 of the NDM-1 coding sequence (SEQ ID NO:88), primer pair 3 results in amplification of a region comprising the nucleotide at position 104 of the OXA-9 coding sequence (SEQ ID NO:89), primer pair 4 results in amplification of a region comprising the nucleotide at position 186 of the OXA-48 coding sequence (SEQ ID NO:90), primer pair 5 results in amplification of a region comprising the nucleotide at position 556 of the CTX-M-9 coding sequence (SEQ ID NO:91), primer pair 6 results in amplification of a region comprising the nucleotide at position 453 of the CTX-M-15 coding sequence (SEQ ID NO:92), primer pair 7 results in amplification of a region comprising the nucleotide at position 223 of the AAC(6′)-lb-cr coding sequence (SEQ ID NO:93) and primer pair 8 results in amplification of a region comprising the nucleotide at position 454 of the AAC(6′)-lb-cr coding sequence (SEQ ID NO:93), or at analogous positions in variants or homologous coding sequences having at least 80%, more preferably at least 90%, more preferably at least 95%, more preferably at least 98%, even more preferably at least 99% sequence identity with the indicated coding sequences.

In the methods described above, the step of performing one or more of the optional PCR reactions comprising primer pairs 9 to 13 preferably results in PCR products, in particular in amplification of regions of interest comprised in the OXA-1 coding sequence (SEQ ID NO:163), the OXA-2 coding sequence (SEQ ID NO:164), the TEM-1 coding sequence (SEQ ID NO:165), the APHA3-la coding sequence (SEQ ID NO:166), and the AAC(3′)-IIae coding sequence (SEQ ID NO:167), or in variants or homologous coding sequences having at least 80%, more preferably at least 90%, more preferably at least 95%, more preferably at least 98%, even more preferably at least 99% sequence identity with the indicated coding sequences in case the respective coding sequences are present in the analyzed sample. Exemplary binding regions of the amplification primers and of the SNPs of interest are shown in FIG. 6.

Particularly preferably, primer pair 9 results in amplification of a region comprising the nucleotide at position 408 of the OXA-1 coding sequence (SEQ ID NO:163), primer pair 10 results in amplification of a region comprising the nucleotide at position 585 of the OXA-2 coding sequence (SEQ ID NO:164), primer pair 11 results in amplification of a region comprising the nucleotide at position 228 of the TEM-1 coding sequence (SEQ ID NO:165), primer pair 12 results in amplification of a region comprising the nucleotide at position 230 of the APHA3-la coding sequence (SEQ ID NO:166), and primer pair 13 results in amplification of a region comprising the nucleotide at position 63 of the AAC(3′)-IIae coding sequence (SEQ ID NO:167), or at analogous positions in variants or homologous coding sequences having at least 80%, more preferably at least 90%, more preferably at least 95%, more preferably at least 98%, even more preferably at least 99% sequence identity with the indicated coding sequences.

In addition to the step A) of performing PCR reactions comprising the indicated primer pairs, the methods of the present invention preferably comprise the additional step of:
B) Allowing extension primers to bind to the PCR products obtained from the PCR reactions of step A), wherein preferably at least one, more preferably exactly one extension primer is binding to each of the PCR products obtained from step A) of the method.

Thus, preferably an extension primer is allowed to bind to the PCR product of primer pair 1, another extension primer is allowed to bind to the PCR product of primer pair 2, another extension primer is allowed to bind to the PCR product of primer pair 3, another extension primer is allowed to bind to the PCR product of primer pair 4, another extension primer is allowed to bind to the PCR product of primer pair 5, another extension primer is allowed to bind to the PCR product of primer pair 6, another extension primer is allowed to bind to the PCR product of primer pair 7 and another extension primer is allowed to bind to the PCR product of primer pair 8. Exemplary binding regions of extension primers are illustrated in FIG. 1.

In embodiments of the present invention in which one or more of the optional PCR reactions of primer pairs 9 to 13 indicated under points (I) to (V) above are performed, preferably an extension primer is allowed to bind to the PCR product of primer pair 9, another extension primer is allowed to bind to the PCR product of primer pair 10, another extension primer is allowed to bind to the PCR product of primer pair 11, another extension primer is allowed to bind to the PCR product of primer pair 12, and another extension primer is allowed to bind to the PCR product of primer pair 13. Exemplary binding regions of extension primers are illustrated in FIG. 6.

Sequences that are preferably comprised by these extension primers are presented in the sequence listing of the present invention and are summarized in the following table 5. The sequences indicated in table 5 are most preferred. However, variants having a sequence identity of at least 80%, more preferably at least 85% more preferably at least 90%, more preferably at least 93% with the sequences indicated in table 5 are also preferable sequences of the present invention.

TABLE 5

| | SEQ | |
|---|---|---|
| | ID | Comprised |
| Primer name | NO: | sequence (5′-3′) |
| KPC-2 extension | 3 | GCCTAACAAGGATGACAAG |
| NDM-1 extension | 50 | CTGAGCGGGTGCATG |
| OXA-9 extension | 53 | GTAATGGCGTCTGCG |
| OXA-48 extension | 56 | GCGGGTAAAAATGCTTGGTT |
| CTX-M-9 extension | 59 | AGAGACACCACCACG |
| CTX-M-15 extension | 18 | TCGGGCGAACGCGGT |
| AAC(6′)-Ib-cr (223) | 64 | GGAAGCGGGGACGGA |
| extension | | |
| AAC(6′)-Ib-cr (454) | 24 | ACACGGCTGGACCAT |
| extension | | |
| OXA-1 extension | 97 | CCAAAAAATTGGATTAAATAAAATCAA |
| OXA-2 extension | 100 | CAGCGCTTGGTCAAGGA |
| TEM-1 extension | 140 | TTAAAGTTCTGCTATGTGG |
| APHA3-Ia extension | 143 | TCGGAAGAGGCATAAAT |
| AAC(3′)-IIae | 109 | GGCATGCACCATCAA |
| extension | | |

Preferably, the following extension primers are allowed to bind to the PCR products obtained from the PCR reactions of step A):
a. KPC-2 extension primer comprising a sequence of SEQ ID NO:3 or a sequence having a sequence identity of at least 80%, more preferably at least 85% more preferably at least 90%, more preferably at least 93% with SEQ ID NO:3,
b. NDM-1 extension primer comprising a sequence of SEQ ID NO:50 or a sequence having a sequence identity of at least 80%, more preferably at least 85% more preferably at least 90%, more preferably at least 93% with SEQ ID NO:50,
c. OXA-9 extension primer comprising a sequence of SEQ ID NO:53 or a sequence having a sequence identity of at least 80%, more preferably at least 85% more preferably at least 90%, more preferably at least 93% with SEQ ID NO:53,
d. OXA-48 extension primer comprising a sequence of SEQ ID NO:56 or a sequence having a sequence identity of at least 80%, more preferably at least 85% more preferably at least 90%, more preferably at least 93% with SEQ ID NO:56,
e. CTX-M-9 extension primer comprising a sequence of SEQ ID NO:59 or a sequence having a sequence identity of at least 80%, more preferably at least 85% more preferably at least 90%, more preferably at least 93% with SEQ ID NO:59,
f. CTX-M-15 extension primer comprising a sequence of SEQ ID NO:18 or a sequence having a sequence identity of at least 80%, more preferably at least 85% more preferably at least 90%, more preferably at least 93% with SEQ ID NO:18,
g. AAC(6′)-lb-cr (223) extension primer comprising a sequence of SEQ ID NO:64 or a sequence having a sequence identity of at least 80%, more preferably at least 85% more preferably at least 90%, more preferably at least 93% with SEQ ID NO:64, and
h. AAC(6′)-lb-cr (454) extension primer comprising a sequence of SEQ ID NO:24 or a sequence having a sequence identity of at least 80%, more preferably at least 85% more preferably at least 90%, more preferably at least 93% with SEQ ID NO:24.

In embodiments of the present invention in which one or more of the optional PCR reactions of primer pairs 9 to 13 indicated under points (I) to (V) above are performed, preferably the following extension primers are allowed to bind to the PCR products obtained from the PCR reactions of step A) that are based on primer pairs 9 to 13 indicated under points (I) to (V) above:
(I) OXA-1 extension primer comprising a sequence of SEQ ID NO:97 or a sequence having a sequence identity of at least 80%, more preferably at least 85% more preferably at least 90%, more preferably at least 93% with SEQ ID NO:97,
(II) OXA-2 extension primer comprising a sequence of SEQ ID NO:100 or a sequence having a sequence identity of at least 80%, more preferably at least 85% more preferably at least 90%, more preferably at least 93% with SEQ ID NO:100,
(III) TEM-1 extension primer comprising a sequence of SEQ ID NO:140 or a sequence having a sequence identity of at least 80%, more preferably at least 85% more preferably at least 90%, more preferably at least 93% with SEQ ID NO:140,
(IV) APHA3-la extension primer comprising a sequence of SEQ ID NO:143 or a sequence having a sequence identity of at least 80%, more preferably at least 85% more preferably at least 90%, more preferably at least 93% with SEQ ID NO:143, and/or
(V) AAC(3′)-IIae extension primer comprising a sequence of SEQ ID NO:109 or a sequence having a sequence identity of at least 80%, more preferably at least 85% more preferably at least 90%, more preferably at least 93% with SEQ ID NO:109.

All primers indicated under points a. to h. and (I) to (V) above may comprise a sequence having at least 80%, more preferably at least 85%, more preferably at least 90%, more preferably at least 93% sequence identity with the sequences of the indicated SEQ ID NOs.

In embodiments of the present invention, the extension primers do not only comprise the sequences indicated in table 5 or variants having a sequence identity of at least 80%, more preferably at least 85% more preferably at least 90%, more preferably at least 93% therewith but even consist of these sequences. However, the present inventors found that it may be advantageous if non-complementary bases are added at the 5′-end of the primer so that each primer has a detectable different mass. Preferably, in addition to the sequences described in table 5 or to variants thereof as indicated above, the extension primers comprise at least 1, more preferably at least 2 non-complementary bases at the 5′-end. However, the number of non-complementary bases at the 5′-end should be limited. Preferably, the extension primers comprise at most 10, more preferably at most 5, more preferably at most 4 non-complementary bases at the 5′-end. It was found that non-complementary bases at the 5′-end are particularly preferred for NDM-1 extension primers, OXA-9 extension primers, OXA-48 extension primers, CTX-M-9 extension primers, AAC(6′)-lb-cr (223) extension primers, TEM-1 extension primers and APHA3-la extension primers.

Sequences that are comprised in most preferred extension primers of the present invention comprising non-complementary bases at the 5′-end are summarized in the following table 6. However, variants having a sequence identity of at least 80%, more preferably at least 85% more preferably at least 90%, more preferably at least 93% with the sequences indicated in table 6 are also preferable sequences of the present invention.

TABLE 6

| | SEQ | |
|---|---|---|
| | ID | Comprised |
| Primer name | NO: | sequence (5′-3′) |
| NDM-1 extension long | 6 | GGGACTGAGCGGGTGCATG |
| OXA-9 extension long | 9 | CTGTAATGGCGTCTGCG |
| OXA-48 extension long | 12 | GGGCGGGTAAAAATGCTTGGTT |
| CTX-M-9 extension long | 15 | TTAGAGACACCACCACG |
| AAC(6′)-Ib-cr (223) | 21 | CGGAAGCGGGGACGGA |
| extension long | | |
| TEM-1 extension long | 103 | GTTAAAGTTCTGCTATGTGG |
| APHA3-Ia extension long | 106 | ATAATCGGAAGAGGCATAAAT |

Preferably, the following extension primers are allowed to bind to the PCR products obtained from the PCR reactions of step A):
a. KPC-2 extension primer comprising a sequence of SEQ ID NO:3 or a sequence having a sequence identity of at least 80%, more preferably at least 85% more preferably at least 90%, more preferably at least 93% with SEQ ID NO:3,
b. NDM-1 extension primer comprising a sequence of SEQ ID NO:6 or a sequence having a sequence identity of at least 80%, more preferably at least 85% more preferably at least 90%, more preferably at least 93% with SEQ ID NO:6,
c. OXA-9 extension primer comprising a sequence of SEQ ID NO:9 or a sequence having a sequence identity of at least 80%, more preferably at least 85% more preferably at least 90%, more preferably at least 93% with SEQ ID NO:9,
d. OXA-48 extension primer comprising a sequence of SEQ ID NO:12 or a sequence having a sequence identity of at least 80%, more preferably at least 85% more preferably at least 90%, more preferably at least 93% with SEQ ID NO:12,
e. CTX-M-9 extension primer comprising a sequence of SEQ ID NO:15 or a sequence having a sequence identity of at least 80%, more preferably at least 85% more preferably at least 90%, more preferably at least 93% with SEQ ID NO:15,
f. CTX-M-15 extension primer comprising a sequence of SEQ ID NO:18 or a sequence having a sequence identity of at least 80%, more preferably at least 85% more preferably at least 90%, more preferably at least 93% with SEQ ID NO:18,
g. AAC(6′)-lb-cr (223) extension primer comprising a sequence of SEQ ID NO:21 or a sequence having a sequence identity of at least 80%, more preferably at least 85% more preferably at least 90%, more preferably at least 93% with SEQ ID NO:21, and
h. AAC(6′)-lb-cr (454) extension primer comprising a sequence of SEQ ID NO:24 or a sequence having a sequence identity of at least 80%, more preferably at least 85% more preferably at least 90%, more preferably at least 93% with SEQ ID NO:24.

In embodiments of the present invention in which one or more of the optional PCR reactions of primer pairs 9 to 13 indicated under points (I) to (V) above are performed, preferably the following extension primers are allowed to bind to the PCR products obtained from the PCR reactions of step A) that are based on primer pairs 9 to 13 indicated under points (I) to (V) above:
(I) OXA-1 extension primer comprising a sequence of SEQ ID NO:97 or a sequence having a sequence identity of at least 80%, more preferably at least 85% more preferably at least 90%, more preferably at least 93% with SEQ ID NO:97,
(II) OXA-2 extension primer comprising a sequence of SEQ ID NO:100 or a sequence having a sequence identity of at least 80%, more preferably at least 85% more preferably at least 90%, more preferably at least 93% with SEQ ID NO:100,
(III) TEM-1 extension primer comprising a sequence of SEQ ID NO:103 or a sequence having a sequence identity of at least 80%, more preferably at least 85% more preferably at least 90%, more preferably at least 93% with SEQ ID NO:103,
(IV) APHA3-la extension primer comprising a sequence of SEQ ID NO:106 or a sequence having a sequence identity of at least 80%, more preferably at least 85% more preferably at least 90%, more preferably at least 93% with SEQ ID NO:106, and/or
(V) AAC(3′)-IIae extension primer comprising a sequence of SEQ ID NO:109 or a sequence having a sequence identity of at least 80%, more preferably at least 85% more preferably at least 90%, more preferably at least 93% with SEQ ID NO:109.

All primers indicated under points a. to h. and (I) to (V) above may comprise a sequence having at least 80%, more preferably at least 85%, more preferably at least 90%, more preferably at least 93% sequence identity with the sequences of the indicated SEQ ID NOs.

Preferably, the extension primers indicated in table 6 do not only comprise the sequences indicated in table 6 or variants having a sequence identity of at least 80%, more preferably at least 85% more preferably at least 90%, more preferably at least 93% therewith but even consist of these sequences.

In addition to step A) of performing PCR reactions comprising the indicated primer pairs and step B) of allowing extension primers to bind to the PCR products obtained from step A), the methods of the present invention preferably comprise the additional step of:
C) Elongating the extension primers at the 3′-end by at least one nucleotide, preferably by exactly one nucleotide.

Preferably, the elongation of all extension primers is performed in a single tube, more preferably in the same tube, in which the PCR reactions have been performed.

Preferably, the methods of the present invention comprise the additional step of:
D) Identifying the at least one nucleotide, preferably the exactly one nucleotide that has been added to the extension primer.

Preferably, the nucleotide is determined by mass spectrometry. Thus, the masses of the elongated extension primers are preferably determined by mass spectrometry. This enables identification of the nucleotide that has been added to the extension primer by comparison of the added mass with the known masses of the different nucleotides.

Preferably, the methods of the present invention comprise the additional step of:
E) Determining information on both the phylogenetic origin and antibiotic resistances of the bacterial strain based on a comparison of the pattern of the determined nucleotides with respective predetermined patterns of bacterial strains, which phylogenetic origin and antibiotic resistances are known.

The PCR reactions of the methods of the present invention described above may additionally comprise further primer pairs. These additional primer pairs may result in amplification of further regions of interest of resistance conferring genes and/or in amplification of regions of interest of genes that do not confer resistance, in particular of house-keeping genes. The present inventors found that the identification of the phylogenetic origin can be particularly preferably improved if the results obtained from analysis of the coding sequences of the antibiotic resistance conferring genes (SEQ ID NOs: 87-93 and optionally one or more of SEQ ID NOs: 163-167) are combined with phylogenetic information obtained by analysis of species-specific phylogeny markers, in particular of house-keeping genes. This is shown exemplarily in the present description for Klebsiella pneumoniae and for Escherichia coli.

The present inventors have found that determination of information on the phylogenetic origin by the method of the present invention can be further improved if PCR reactions are performed that comprise further primer pairs in addition to primer pairs 1 to 8 and to the optional primer pairs 9 to 13 described above.

Sequences that are preferably comprised by these additional PCR primers are presented in the sequence listing of the present invention and are summarized in the following table 7. The sequences indicated in table 7 are most preferred. However, variants having a sequence identity of at least 80%, more preferably at least 85% more preferably at least 90%, more preferably at least 94% with the sequences indicated in table 7 are also preferable sequences of the present invention.

TABLE 7

| | SEQ | |
|---|---|---|
| | ID | Comprised |
| Primer name | NO: | sequence (5′-3′) |
| gapA forward | 67 | TGGCCCGTCCAAAGACAACA |
| gapA reverse | 68 | ATCAGGCCTTCAACGATACC |
| infB (1608) forward | 73 | TGAAGAACGAACTGTCCCAG |
| infB (1608) reverse | 74 | GTCCAGCAGGTCGTCGATG |
| mdh (633) forward | 76 | GGGTCACCATTCTGCCTTTA |
| mdh (633) reverse | 77 | CGCCCGCTTTCGCTTCCAC |
| phoE (732) forward | 79 | ACAATATCTACCTGGCGACC |
| phoE (732) reverse | 80 | GTTGAAGTAGTAGGTCAGGC |
| phoE (867) forward | 82 | TCCCTCGGCTATGTGCTGT |
| phoE (867) reverse | 83 | CGAAGGCGTTCATGTTTTTG |
| rpoB (1819) forward | 84 | TGATTAACTCCCTGTCCGTG |
| rpoB (1819) reverse | 85 | CCTTCTTCGATAGCAGACAG |
| gyrB forward | 146 | TCACCGTCAGATCTACGAAC |
| gyrB reverse | 147 | CATGGTGCCGGTTTTTTCAG |
| adk (348) forward | 149 | TTCTGGAATTCGACGTACCG |
| adk (348) reverse | 150 | TGATAAACACGACCAGACGG |
| adk (456) forward | 151 | ATTCAACCCGCCGAAAGTAG |
| adk (456) reverse | 152 | CTTCCTGATCATCTTTACGG |
| icd (243) forward | 153 | CGGTGAAAAATCCACACAGG |
| icd (243) reverse | 154 | ATGGCAACGCGATATTCACG |
| icd (606) forward | 155 | GAAAATTCGCTTCCCGGAAC |
| icd (606) reverse | 156 | GAACCAGACGTTTGGTGCC |
| purA forward | 157 | TATCGGGCCTGCTTATGAAG |
| purA reverse | 158 | TTGTCGAAAAGGTCGCCAAC |
| lexA forward | 159 | AGCCGAATGCTGATTTCCTG |
| lexA reverse | 160 | CCTGAGTTTTATGCACTGCC |
| rpoH forward | 161 | GCCTGATGAAAGCAGTGCG |
| rpoH reverse | 162 | ATTCGTGGATCTCTGCTTTG |

By using a combination of corresponding forward and reverse primers, DNA fragments containing the SNP positions of the present invention can be excellently amplified, in particular as PCR products.

Primers of SEQ ID NOs: 67, 68, 73, 74, 76, 77, 79, 80, 82-85 shown in table 7 above are particularly suitable for determining additional phylogenetic information on Klebsiella, in particular on Klebsiella pneumoniae. Primers of SEQ ID NOs: 146, 147, 149-162 shown in table 7 above are particularly suitable for determining additional phylogenetic information on Escherichia, in particular on Escherichia coli.

In embodiments of the present invention, the amplifying primers do not only comprise the sequences indicated in table 7 or variants having a sequence identity of at least 80%, more preferably at least 85% more preferably at least 90%, more preferably at least 94% therewith but even consist of these sequences. However, the present inventors found that the amplification is advantageously balanced if the amplification primers additionally comprise nucleotides at the 5′-end. Preferably, in addition to the sequences described in table 7 or to variants thereof as indicated above, the amplification primers comprise at least 5, more preferably at least 8, more preferably at least 9, more preferably at least 10 and even more preferably exactly 10 additional nucleotides at the 5′-end. The number of additional nucleotides at the 5′-end should be limited and therefore the amplification primers preferably comprise at most 20, more preferably at most 15, more preferably at most 12, more preferably at most 11 and even more preferably at most 10 additional nucleotides at the 5′-end. Sequences that are comprised in most preferred amplification primers of the present invention are summarized in the following table 8. However, variants having a sequence identity of at least 80%, more preferably at least 85% more preferably at least 90%, more preferably at least 95% with the sequences indicated in table 8 are also preferable sequences of the present invention.

TABLE 8

| Primer name | SEQ ID NO: | Comprised sequence (5′-3′) |
|---|---|---|
| gapA long forward | 25 | ACGTTGGATGTGGCCCGTCCAAAGACAACA |
| gapA long reverse | 26 | ACGTTGGATGATCAGGCCTTCAACGATACC |
| infB (1608) long forward | 31 | ACGTTGGATGTGAAGAACGAACTGTCCCAG |
| infB (1608) long reverse | 32 | ACGTTGGATGGTCCAGCAGGTCGTCGATG |
| mdh (633) long forward | 34 | ACGTTGGATGGGGTCACCATTCTGCCTTTA |
| mdh (633) long reverse | 35 | ACGTTGGATGCGCCCGCTTTCGCTTCCAC |
| phoE (732) long forward | 37 | ACGTTGGATGACAATATCTACCTGGCGACC |
| phoE (732) long reverse | 38 | ACGTTGGATGGTTGAAGTAGTAGGTCAGGC |
| phoE (867) long forward | 40 | ACGTTGGATGTCCCTCGGCTATGTGCTGT |
| phoE (867) long reverse | 41 | ACGTTGGATGCGAAGGCGTTCATGTTTTTG |
| rpoB (1819) long forward | 43 | ACGTTGGATGTGATTAACTCCCTGTCCGTG |
| rpoB (1819) long reverse | 44 | ACGTTGGATGCCTTCTTCGATAGCAGACAG |
| gyrB long forward | 110 | ACGTTGGATGTCACCGTCAGATCTACGAAC |
| gyrB long reverse | 111 | ACGTTGGATGCATGGTGCCGGTTTTTTCAG |
| adk (348) long forward | 113 | ACGTTGGATGTTCTGGAATTCGACGTACCG |
| adk (348) long reverse | 114 | ACGTTGGATGTGATAAACACGACCAGACGG |
| adk (456) long forward | 116 | ACGTTGGATGATTCAACCCGCCGAAAGTAG |
| adk (456) long reverse | 117 | ACGTTGGATGCTTCCTGATCATCTTTACGG |
| icd (243) long forward | 119 | ACGTTGGATGCGGTGAAAAATCCACACAGG |
| icd (243) long reverse | 120 | ACGTTGGATGATGGCAACGCGATATTCACG |
| icd (606) long forward | 122 | ACGTTGGATGGAAAATTCGCTTCCCGGAAC |
| icd (606) long reverse | 123 | ACGTTGGATGGAACCAGACGTTTGGTGCC |
| purA long forward | 125 | ACGTTGGATGTATCGGGCCTGCTTATGAAG |
| purA long reverse | 126 | ACGTTGGATGTTGTCGAAAAGGTCGCCAAC |
| lexA long forward | 128 | ACGTTGGATGAGCCGAATGCTGATTTCCTG |
| lexA long reverse | 129 | ACGTTGGATGCCTGAGTTTTATGCACTGCC |
| rpoH long forward | 131 | ACGTTGGATGGCCTGATGAAAGCAGTGCG |
| rpoH long reverse | 132 | ACGTTGGATGATTCGTGGATCTCTGCTTTG |

Primers of SEQ ID NOs: 25, 26, 31, 32, 34, 35, 37, 38, 40, 41, 43, 44 shown in table 8 above are particularly suitable for determining additional phylogenetic information on Klebsiella, in particular on Klebsiella pneumoniae. Primers of SEQ ID NOs: 110, 111, 113, 114, 116, 117, 119, 120, 122, 123, 125, 126, 128, 129, 131, 132 shown in table 8 above are particularly suitable for determining additional phylogenetic information on Escherichia, in particular on Escherichia coll.

Preferably, the amplifying primers do not only comprise the sequences indicated in table 8 or variants having a sequence identity of at least 80%, more preferably at least 85% more preferably at least 90%, more preferably at least 95% therewith but even consist of these sequences.

Preferably, the PCR reactions of step A) of the methods of the present invention described above for determining in a bacterial sample information on both the phylogenetic origin and the antibiotic resistances of bacterial strains additionally comprise at least one, more preferably at least two, more preferably at least three, more preferably at least four, more preferably at least five, more preferably all six of the following primer pairs:
i. Primer pair 14 consisting of a forward primer comprising a sequence of SEQ ID NO:67 or a sequence having at least 80%, preferably at least 85%, more preferably at least 90%, more preferably at least 94% sequence identity therewith, and a reverse primer comprising a sequence of SEQ ID NO:68 or a sequence having at least 80%, preferably at least 85%, more preferably at least 90%, more preferably at least 94% sequence identity therewith,
j. Primer pair 15 consisting of a forward primer comprising a sequence of SEQ ID NO:73 or a sequence having at least 80%, preferably at least 85%, more preferably at least 90%, more preferably at least 94% sequence identity therewith, and a reverse primer comprising a sequence of SEQ ID NO:74 or a sequence having at least 80%, preferably at least 85%, more preferably at least 90%, more preferably at least 94% sequence identity therewith,
k. Primer pair 16 consisting of a forward primer comprising a sequence of SEQ ID NO:76 or a sequence having at least 80%, preferably at least 85%, more preferably at least 90%, more preferably at least 94% sequence identity therewith, and a reverse primer comprising a sequence of SEQ ID NO:77 or a sequence having at least 80%, preferably at least 85%, more preferably at least 90%, more preferably at least 94% sequence identity therewith,
l. Primer pair 17 consisting of a forward primer comprising a sequence of SEQ ID NO:79 or a sequence having at least 80%, preferably at least 85%, more preferably at least 90%, more preferably at least 94% sequence identity therewith, and a reverse primer comprising a sequence of SEQ ID NO:80 or a sequence having at least 80%, preferably at least 85%, more preferably at least 90%, more preferably at least 94% sequence identity therewith,
m. Primer pair 18 consisting of a forward primer comprising a sequence of SEQ ID NO:82 or a sequence having at least 80%, preferably at least 85%, more preferably at least 90%, more preferably at least 94% sequence identity therewith, and a reverse primer comprising a sequence of SEQ ID NO:83 or a sequence having at least 80%, preferably at least 85%, more preferably at least 90%, more preferably at least 94% sequence identity therewith, and
n. Primer pair 19 consisting of a forward primer comprising a sequence of SEQ ID NO:84 or a sequence having at least 80%, preferably at least 85%, more preferably at least 90%, more preferably at least 94% sequence identity therewith, and a reverse primer comprising a sequence of SEQ ID NO:85 or a sequence having at least 80%, preferably at least 85%, more preferably at least 90%, more preferably at least 94% sequence identity therewith.

Additionally or alternatively to primer pairs 14 to 19 described above, the PCR reactions of step A) of the methods of the present invention described above for determining in a bacterial sample information on both the phylogenetic origin and the antibiotic resistances of bacterial strains preferably comprise at least one, more preferably at least two, more preferably at least three, more preferably at least four, more preferably at least five, more preferably at least six, more preferably at least seven, more preferably all eight of the following primer pairs:
(VI) Primer pair 20 consisting of a forward primer comprising a sequence of SEQ ID NO:146 or a sequence having at least 80%, preferably at least 85%, more preferably at least 90%, more preferably at least 94% sequence identity therewith, and a reverse primer comprising a sequence of SEQ ID NO:147 or a sequence having at least 80%, preferably at least 85%, more preferably at least 90%, more preferably at least 94% sequence identity therewith,
(VII) Primer pair 21 consisting of a forward primer comprising a sequence of SEQ ID NO:149 or a sequence having at least 80%, preferably at least 85%, more preferably at least 90%, more preferably at least 94% sequence identity therewith, and a reverse primer comprising a sequence of SEQ ID NO:150 or a sequence having at least 80%, preferably at least 85%, more preferably at least 90%, more preferably at least 94% sequence identity therewith,
(VIII) Primer pair 22 consisting of a forward primer comprising a sequence of SEQ ID NO:151 or a sequence having at least 80%, preferably at least 85%, more preferably at least 90%, more preferably at least 94% sequence identity therewith, and a reverse primer comprising a sequence of SEQ ID NO:152 or a sequence having at least 80%, preferably at least 85%, more preferably at least 90%, more preferably at least 94% sequence identity therewith,
(IX) Primer pair 23 consisting of a forward primer comprising a sequence of SEQ ID NO:153 or a sequence having at least 80%, preferably at least 85%, more preferably at least 90%, more preferably at least 94% sequence identity therewith, and a reverse primer comprising a sequence of SEQ ID NO:154 or a sequence having at least 80%, preferably at least 85%, more preferably at least 90%, more preferably at least 94% sequence identity therewith,
(X) Primer pair 24 consisting of a forward primer comprising a sequence of SEQ ID NO:155 or a sequence having at least 80%, preferably at least 85%, more preferably at least 90%, more preferably at least 94% sequence identity therewith, and a reverse primer comprising a sequence of SEQ ID NO:156 or a sequence having at least 80%, preferably at least 85%, more preferably at least 90%, more preferably at least 94% sequence identity therewith,
(XI) Primer pair 25 consisting of a forward primer comprising a sequence of SEQ ID NO:157 or a sequence having at least 80%, preferably at least 85%, more preferably at least 90%, more preferably at least 94% sequence identity therewith, and a reverse primer comprising a sequence of SEQ ID NO:158 or a sequence having at least 80%, preferably at least 85%, more preferably at least 90%, more preferably at least 94% sequence identity therewith,
(XII) Primer pair 26 consisting of a forward primer comprising a sequence of SEQ ID NO:159 or a sequence having at least 80%, preferably at least 85%, more preferably at least 90%, more preferably at least 94% sequence identity therewith, and a reverse primer comprising a sequence of SEQ ID NO:160 or a sequence having at least 80%, preferably at least 85%, more preferably at least 90%, more preferably at least 94% sequence identity therewith, and/or
(XIII) Primer pair 27 consisting of a forward primer comprising a sequence of SEQ ID NO:161 or a sequence having at least 80%, preferably at least 85%, more preferably at least 90%, more preferably at least 94% sequence identity therewith, and a reverse primer comprising a sequence of SEQ ID NO:162 or a sequence having at least 80%, preferably at least 85%, more preferably at least 90%, more preferably at least 94% sequence identity therewith.

Primer pairs 14 to 19 as indicated under points i. to n. above are particularly suitable for determining additional phylogenetic information on Klebsiella, in particular on Klebsiella pneumoniae. Primer pairs 20 to 27 as indicated under points (VI) to (XIII) above are particularly suitable for determining additional phylogenetic information on Escherichia, in particular on Escherichia coli.

All primers of the primer pairs indicated under points i. to n. and (VI) to (XIII) above may comprise a sequence having at least 80%, more preferably at least 85%, more preferably at least 90%, more preferably at least 94% sequence identity with the sequences of the indicated SEQ ID NOs.

The method of the present invention comprises the step of performing PCR reactions comprising primer pairs 1 to 8 as described above under points a. to h. Preferably, PCR reactions are performed comprising one or more of primer pairs 9 to 13 described above under points (I) to (V) and/or one or more of primer pairs 14 to 19 described above under points i. to n. and/or one or more of primer pairs 20 to 27 described above under points (VI) to (XIII).

Thus, in exemplary embodiments of the present invention PCR reactions are performed comprising primer pairs 1 to 8 as described above under points a. to h.

In exemplary embodiments of the present invention PCR reactions are performed comprising primer pairs 1 to 8 as described above under points a. to h. and additionally one or more, preferably all of primer pairs 9 to 13 described above under points (I) to (V).

In exemplary embodiments of the present invention PCR reactions are performed comprising primer pairs 1 to 8 as described above under points a. to h. and additionally one or more, preferably all of primer pairs 14 to 19 described above under points i. to n.

In exemplary embodiments of the present invention PCR reactions are performed comprising primer pairs 1 to 8 as described above under points a. to h. and additionally one or more, preferably all of primer pairs 20 to 27 described above under points (VI) to (XIII).

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 14 to 19 described above under points i. to n.

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 20 to 27 described above under points (VI) to (XIII).

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 14 to 19 described above under points i. to n. and additionally one or more, preferably all of primer pairs 20 to 27 described above under points (VI) to (XIII).

The PCR reactions of the methods of the present invention described above may additionally comprise further primer pairs. These additional primer pairs may result in amplification of further regions of interest of resistance conferring genes and/or in amplification of regions of interest of genes that do not confer resistance, in particular of house-keeping genes.

According to the present invention, the PCR reaction of each primer pair may be performed in a separate tube. Thus, for example, 14 separate PCR reactions may be performed with primer pairs 1 to 8 and 14 to 19 described above or 21 separate PCR reactions may be performed with primer pairs 1 to 13 and 20 to 27 described above. However, it is preferable that the number of separate PCR reactions is reduced in order to save time and resources. Preferably, all PCR reactions are performed in a single tube.

In embodiments of the present invention, the forward and reverse primers of primer pairs 14 to 27 do not only comprise the sequences of SEQ ID NOs: 67, 68, 73, 74, 76, 77, 79, 80, 82-85, 146, 147, 149-162 as indicated above or variants having a sequence identity of at least 80%, more preferably at least 85% more preferably at least 90%, more preferably at least 94% therewith but even consist of these sequences. However, the present inventors found that the amplification is advantageously balanced if the forward and reverse primers additionally comprise nucleotides at the 5′-end. Furthermore, this increases the masses of unused primers (out of the mass range on the spectra).

Therefore, the PCR reactions of step A) of the methods of the present invention described above for determining in a bacterial sample information on both the phylogenetic origin and the antibiotic resistances of bacterial strains additionally comprise at least one, more preferably at least two, more preferably at least three, more preferably at least four, more preferably at least five, more preferably all six of the following primer pairs:
i. Primer pair 14 consisting of a forward primer comprising a sequence of SEQ ID NO:25 or a sequence having at least 80%, preferably at least 85%, more preferably at least 90%, more preferably at least 95% sequence identity therewith, and a reverse primer comprising a sequence of SEQ ID NO:26 or a sequence having at least 80%, preferably at least 85%, more preferably at least 90%, more preferably at least 95% sequence identity therewith,
j. Primer pair 15 consisting of a forward primer comprising a sequence of SEQ ID NO:31 or a sequence having at least 80%, preferably at least 85%, more preferably at least 90%, more preferably at least 95% sequence identity therewith, and a reverse primer comprising a sequence of SEQ ID NO:32 or a sequence having at least 80%, preferably at least 85%, more preferably at least 90%, more preferably at least 95% sequence identity therewith,
k. Primer pair 16 consisting of a forward primer comprising a sequence of SEQ ID NO:34 or a sequence having at least 80%, preferably at least 85%, more preferably at least 90%, more preferably at least 95% sequence identity therewith, and a reverse primer comprising a sequence of SEQ ID NO:35 or a sequence having at least 80%, preferably at least 85%, more preferably at least 90%, more preferably at least 95% sequence identity therewith,
l. Primer pair 17 consisting of a forward primer comprising a sequence of SEQ ID NO:37 or a sequence having at least 80%, preferably at least 85%, more preferably at least 90%, more preferably at least 95% sequence identity therewith, and a reverse primer comprising a sequence of SEQ ID NO:38 or a sequence having at least 80%, preferably at least 85%, more preferably at least 90%, more preferably at least 95% sequence identity therewith,
m. Primer pair 18 consisting of a forward primer comprising a sequence of SEQ ID NO:40 or a sequence having at least 80%, preferably at least 85%, more preferably at least 90%, more preferably at least 95% sequence identity therewith, and a reverse primer comprising a sequence of SEQ ID NO:41 or a sequence having at least 80%, preferably at least 85%, more preferably at least 90%, more preferably at least 95% sequence identity therewith, and
n. Primer pair 19 consisting of a forward primer comprising a sequence of SEQ ID NO:43 or a sequence having at least 80%, preferably at least 85%, more preferably at least 90%, more preferably at least 95% sequence identity therewith, and a reverse primer comprising a sequence of SEQ ID NO:44 or a sequence having at least 80%, preferably at least 85%, more preferably at least 90%, more preferably at least 95% sequence identity therewith.

Additionally or alternatively to primer pairs 14 to 19 described above, the PCR reactions of step A) of the methods of the present invention described above for determining in a bacterial sample information on both the phylogenetic origin and the antibiotic resistances of bacterial strains preferably comprise at least one, more preferably at least two, more preferably at least three, more preferably at least four, more preferably at least five, more preferably at least six, more preferably at least seven, more preferably all eight of the following primer pairs:
(VI) Primer pair 20 consisting of a forward primer comprising a sequence of SEQ ID NO:110 or a sequence having at least 80%, preferably at least 85%, more preferably at least 90%, more preferably at least 95% sequence identity therewith, and a reverse primer comprising a sequence of SEQ ID NO:111 or a sequence having at least 80%, preferably at least 85%, more preferably at least 90%, more preferably at least 95% sequence identity therewith,
(VII) Primer pair 21 consisting of a forward primer comprising a sequence of SEQ ID NO:113 or a sequence having at least 80%, preferably at least 85%, more preferably at least 90%, more preferably at least 95% sequence identity therewith, and a reverse primer comprising a sequence of SEQ ID NO:114 or a sequence having at least 80%, preferably at least 85%, more preferably at least 90%, more preferably at least 95% sequence identity therewith,
(VIII) Primer pair 22 consisting of a forward primer comprising a sequence of SEQ ID NO:116 or a sequence having at least 80%, preferably at least 85%, more preferably at least 90%, more preferably at least 95% sequence identity therewith, and a reverse primer comprising a sequence of SEQ ID NO:117 or a sequence having at least 80%, preferably at least 85%, more preferably at least 90%, more preferably at least 95% sequence identity therewith,
(IX) Primer pair 23 consisting of a forward primer comprising a sequence of SEQ ID NO:119 or a sequence having at least 80%, preferably at least 85%, more preferably at least 90%, more preferably at least 95% sequence identity therewith, and a reverse primer comprising a sequence of SEQ ID NO:120 or a sequence having at least 80%, preferably at least 85%, more preferably at least 90%, more preferably at least 95% sequence identity therewith,
(X) Primer pair 24 consisting of a forward primer comprising a sequence of SEQ ID NO:122 or a sequence having at least 80%, preferably at least 85%, more preferably at least 90%, more preferably at least 95% sequence identity therewith, and a reverse primer comprising a sequence of SEQ ID NO:123 or a sequence having at least 80%, preferably at least 85%, more preferably at least 90%, more preferably at least 95% sequence identity therewith,
(XI) Primer pair 25 consisting of a forward primer comprising a sequence of SEQ ID NO:125 or a sequence having at least 80%, preferably at least 85%, more preferably at least 90%, more preferably at least 95% sequence identity therewith, and a reverse primer comprising a sequence of SEQ ID NO:126 or a sequence having at least 80%, preferably at least 85%, more preferably at least 90%, more preferably at least 95% sequence identity therewith,
(XII) Primer pair 26 consisting of a forward primer comprising a sequence of SEQ ID NO:128 or a sequence having at least 80%, preferably at least 85%, more preferably at least 90%, more preferably at least 95% sequence identity therewith, and a reverse primer comprising a sequence of SEQ ID NO:129 or a sequence having at least 80%, preferably at least 85%, more preferably at least 90%, more preferably at least 95% sequence identity therewith, and/or
(XIII) Primer pair 27 consisting of a forward primer comprising a sequence of SEQ ID NO:131 or a sequence having at least 80%, preferably at least 85%, more preferably at least 90%, more preferably at least 95% sequence identity therewith, and a reverse primer comprising a sequence of SEQ ID NO:132 or a sequence having at least 80%, preferably at least 85%, more preferably at least 90%, more preferably at least 95% sequence identity therewith.

Primer pairs 14 to 19 as indicated under points i. to n. above are particularly suitable for determining additional phylogenetic information on Klebsiella, in particular on Klebsiella pneumoniae. Primer pairs 20 to 27 as indicated under points (VI) to (XIII) above are particularly suitable for determining additional phylogenetic information on Escherichia, in particular on Escherichia coli.

All primers of the primer pairs indicated under points i. to n. and (VI) to (XIII) above may comprise a sequence having at least 80%, more preferably at least 85%, more preferably at least 90%, more preferably at least 95% sequence identity with the sequences of the indicated SEQ ID NOs.

The method of the present invention comprises the step of performing PCR reactions comprising primer pairs 1 to 8 as described above under points a. to h. Preferably, PCR reactions are performed comprising one or more of primer pairs 9 to 13 described above under points (I) to (V) and/or one or more of primer pairs 14 to 19 described above under points i. to n. and/or one or more of primer pairs 20 to 27 described above under points (VI) to (XIII).

Thus, in exemplary embodiments of the present invention PCR reactions are performed comprising primer pairs 1 to 8 as described above under points a. to h.

In exemplary embodiments of the present invention PCR reactions are performed comprising primer pairs 1 to 8 as described above under points a. to h. and additionally one or more, preferably all of primer pairs 9 to 13 described above under points (I) to (V).

In exemplary embodiments of the present invention PCR reactions are performed comprising primer pairs 1 to 8 as described above under points a. to h. and additionally one or more, preferably all of primer pairs 14 to 19 described above under points i. to n.

In exemplary embodiments of the present invention PCR reactions are performed comprising primer pairs 1 to 8 as described above under points a. to h. and additionally one or more, preferably all of primer pairs 20 to 27 described above under points (VI) to (XIII).

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 14 to 19 described above under points i. to n.

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 20 to 27 described above under points (VI) to (XIII).

In exemplary embodiments of the present invention PCR reactions are performed comprising primer pairs 1 to 8 as described above under points a. to h and additionally one or more, preferably all of primer pairs 9 to 13 described above under points (I) to (V) and additionally one or more, preferably all of primer pairs 14 to 19 described above under points i. to n. and additionally one or more, preferably all of primer pairs 20 to 27 described above under points (VI) to (XIII).

The PCR reactions of the methods of the present invention described above may additionally comprise further primer pairs. These additional primer pairs may result in amplification of further regions of interest of resistance conferring genes and/or in amplification of regions of interest of genes that do not confer resistance, in particular of house-keeping genes.

According to the present invention, the PCR reaction of each primer pair may be performed in a separate tube. Thus, for example, 14 separate PCR reactions may be performed with primer pairs 1 to 8 and 14 to 19 described above or 21 separate PCR reactions may be performed with primer pairs 1 to 13 and 20 to 27 described above. However, it is preferable that the number of separate PCR reactions is reduced in order to save time and resources. Preferably, all PCR reactions are performed in a single tube.

In preferred embodiments of the present invention, the forward and reverse primers do not only comprise the sequences of SEQ ID NOs: 25, 26, 31, 32, 34, 35, 37, 38, 40, 41, 43, 44, 110, 111, 113, 114, 116, 117, 119, 120, 122, 123, 125, 126, 128, 129, 131, 132 as indicated above or variants having a sequence identity of at least 80%, more preferably at least 85% more preferably at least 90%, more preferably at least 95% therewith but even consist of these sequences.

Preferably, primer pair 14 results in amplification of a region comprising the nucleotide at position 420 and at position 456 of the gapA coding sequence (SEQ ID NO:28), primer pair 15 results in amplification of a region comprising the nucleotide at position 1608 of the infB coding sequence (SEQ ID NO:29), primer pair 16 results in amplification of a region comprising the nucleotide at position 633 of the mdh coding sequence (SEQ ID NO:70), primer pair 17 results in amplification of a region comprising the nucleotide at position 732 of the phoE coding sequence (SEQ ID NO:71), primer pair 18 results in amplification of a region comprising the nucleotide at position 867 of the phoE coding sequence (SEQ ID NO:71) and primer pair 19 results in amplification of a region comprising the nucleotide at position 1819 of the rpoB coding sequence (SEQ ID NO:94), or at analogous positions in variants or homologous coding sequences having at least 80%, more preferably at least 90%, more preferably at least 95%, more preferably at least 98%, even more preferably at least 99% sequence identity with the indicated coding sequences. Exemplary binding regions of the amplifications primers and of the SNPs of interest are shown in FIG. 2.

Preferably, primer pair 20 results in amplification of a region comprising the nucleotide at position 456 of the gyrB coding sequence (SEQ ID NO:168), primer pair 21 results in amplification of a region comprising the nucleotide at position 348 of the adk coding sequence (SEQ ID NO:169), primer pair 22 results in amplification of a region comprising the nucleotide at position 456 of the adk coding sequence (SEQ ID NO:169), primer pair 23 results in amplification of a region comprising the nucleotide at position 243 of the icd coding sequence (SEQ ID NO:170), primer pair 24 results in amplification of a region comprising the nucleotide at position 606 of the icd coding sequence (SEQ ID NO:170), primer pair 25 results in amplification of a region comprising the nucleotide at position 426 of the purA coding sequence (SEQ ID NO:171), primer pair 26 results in amplification of a region comprising the nucleotide at position 384 of the lexA coding sequence (SEQ ID NO:172) and primer pair 27 results in amplification of a region comprising the nucleotide at position 279 of the rpoH coding sequence (SEQ ID NO:173), or at analogous positions in variants or homologous coding sequences having at least 80%, more preferably at least 90%, more preferably at least 95%, more preferably at least 98%, even more preferably at least 99% sequence identity with the indicated coding sequences. Exemplary binding regions of the amplifications primers and of the SNPs of interest are shown in FIG. 7.

In step B) of the methods of the present invention, extension primers are preferably allowed to bind to the PCR products obtained from the PCR reactions of primer pairs 14 to 27, wherein preferably at least one, more preferably exactly one extension primer is binding to each of the obtained PCR products. Particularly preferred, exactly one extension primer is binding to the PCR products obtained from the PCR reactions of primer pairs 15 to 27 and exactly two extension primers are binding to the PCR products obtained from the PCR reactions of primer pair 14. Exemplary binding regions of extension primers are shown in FIG. 2 for primer pairs 14 to 19 and in FIG. 7 for primer pairs 20 to 27.

Sequences that are preferably comprised by these extension primers are presented in the sequence listing of the present invention and are summarized in the following table 9. The sequences indicated in table 9 are most preferred. However, variants having a sequence identity of at least 80%, more preferably at least 85% more preferably at least 90%, more preferably at least 93% with the sequences indicated in table 9 are also preferable sequences of the present invention. The letter “I” in the sequences in table 9 indicates inosine.

TABLE 9

| Primer name | SEQ ID NO: | Comprised sequence (5′-3′) |
|---|---|---|
| gapA (420) extension | 69 | GGAAACGATGTCCTGGCC |
| gapA (456) extension | 72 | CGCITCCTGCACCAC |
| infB (1608) extension | 75 | GCGGCGAGAGCCAGTT |
| mdh (633) extension | 78 | ATTCAIAAIGCCGGTAC |
| phoE (732) extension | 81 | GCTGATCGGGGTCAT |
| phoE (867) extension | 42 | GATATIGAAGGGGTGGG |
| rpoB (1819) extension | 86 | CICCGTATCGTAAAGTGACC |
| gyrB extension | 148 | CACGGTGTACCGCAGGC |
| adk (348) extension primer | 115 | GTACCGGACGAACTGAT |
| adk (456) extension primer | 118 | GCAAAGACGACGTTACCGGTGAAGA |
| icd (243) extension primer | 121 | CAGGTTTATGGTCAGGA |
| icd (606) extension primer | 124 | GGTATCGGTATTAAGCCGTGTTC |
| purA extension primer | 127 | GCCTGCTTATGAAGATAAAGT |
| lexA extension primer | 130 | ATATCGGCATTATGGATGG |
| rpoH extension primer | 133 | TGAAAGCAGTGCGCCGTTT |

Preferably, at least one, more preferably at least two, more preferably at least three, more preferably at least four, more preferably at least five, more preferably at least six, more preferably all seven of the following extension primers are allowed to bind to the PCR products obtained from the PCR reactions of step A) in addition to the extension primers of the antibiotic resistance markers as indicated above:
i. gapA (420) extension primer comprising a sequence of SEQ ID NO:69 or a sequence having a sequence identity of at least 80%, more preferably at least 85% more preferably at least 90%, more preferably at least 93% with SEQ ID NO:69,
j. gapA (456) extension primer comprising a sequence of SEQ ID NO:72 or a sequence having a sequence identity of at least 80%, more preferably at least 85% more preferably at least 90%, more preferably at least 93% with SEQ ID NO:72,
k. infB (1608) extension primer comprising a sequence of SEQ ID NO:75 or a sequence having a sequence identity of at least 80%, more preferably at least 85% more preferably at least 90%, more preferably at least 93% with SEQ ID NO:75,
l. mdh (633) extension primer comprising a sequence of SEQ ID NO:78 or a sequence having a sequence identity of at least 80%, more preferably at least 85% more preferably at least 90%, more preferably at least 93% with SEQ ID NO:78,
m. phoE (732) extension primer comprising a sequence of SEQ ID NO:81 or a sequence having a sequence identity of at least 80%, more preferably at least 85% more preferably at least 90%, more preferably at least 93% with SEQ ID NO:81,
n. phoE (867) extension primer comprising a sequence of SEQ ID NO:42 or a sequence having a sequence identity of at least 80%, more preferably at least 85% more preferably at least 90%, more preferably at least 93% with SEQ ID NO:42,
o. rpoB (1819) extension primer comprising a sequence of SEQ ID NO:86 or a sequence having a sequence identity of at least 80%, more preferably at least 85% more preferably at least 90%, more preferably at least 93% with SEQ ID NO:86.

Preferably, gapA (420) extension primer and gapA (456) extension primer are binding to the PCR product obtained from the PCR reaction comprising primer pair 14, infB (1608) extension primer is binding to the PCR product obtained from the PCR reaction comprising primer pair 15, mdh (633) extension primer is binding to the PCR product obtained from the PCR reaction comprising primer pair 16, phoE (732) extension primer is binding to the PCR product obtained from the PCR reaction comprising primer pair 17, phoE (867) extension primer is binding to the PCR product obtained from the PCR reaction comprising primer pair 18 and rpoB (1819) extension primer is binding to the PCR product obtained from the PCR reaction comprising primer pair 19.

Additionally or alternatively to the extension primers described under points i. to o. above, preferably at least one, more preferably at least two, more preferably at least three, more preferably at least four, more preferably at least five, more preferably at least six, more preferably at least seven, more preferably all eight of the following extension primers are allowed to bind to the PCR products obtained from the PCR reactions of step A) in addition to the extension primers of the antibiotic resistance markers as indicated above:
(VI) gyrB extension primer comprising a sequence of SEQ ID NO:148 or a sequence having a sequence identity of at least 80%, more preferably at least 85% more preferably at least 90%, more preferably at least 93% with SEQ ID NO:148,
(VII) adk (348) extension primer comprising a sequence of SEQ ID NO:115 or a sequence having a sequence identity of at least 80%, more preferably at least 85% more preferably at least 90%, more preferably at least 93% with SEQ ID NO:115,
(VIII) adk (456) extension primer comprising a sequence of SEQ ID NO:118 or a sequence having a sequence identity of at least 80%, more preferably at least 85% more preferably at least 90%, more preferably at least 93% with SEQ ID NO:118,
(IX) icd (243) extension primer comprising a sequence of SEQ ID NO:121 or a sequence having a sequence identity of at least 80%, more preferably at least 85% more preferably at least 90%, more preferably at least 93% with SEQ ID NO:121,
(X) icd (606) extension primer comprising a sequence of SEQ ID NO:124 or a sequence having a sequence identity of at least 80%, more preferably at least 85% more preferably at least 90%, more preferably at least 93% with SEQ ID NO:124,
(XI) purA extension primer comprising a sequence of SEQ ID NO:127 or a sequence having a sequence identity of at least 80%, more preferably at least 85% more preferably at least 90%, more preferably at least 93% with SEQ ID NO:127,
(XII) lexA extension primer comprising a sequence of SEQ ID NO:130 or a sequence having a sequence identity of at least 80%, more preferably at least 85% more preferably at least 90%, more preferably at least 93% with SEQ ID NO:130,
(XIII) rpoH extension primer comprising a sequence of SEQ ID NO:133 or a sequence having a sequence identity of at least 80%, more preferably at least 85% more preferably at least 90%, more preferably at least 93% with SEQ ID NO:133.

Preferably, gyrB extension primer is binding to the PCR product obtained from the PCR reaction comprising primer pair 20, adk (348) extension primer is binding to the PCR product obtained from the PCR reaction comprising primer pair 21, adk (456) extension primer is binding to the PCR product obtained from the PCR reaction comprising primer pair 22, icd (243) extension primer is binding to the PCR product obtained from the PCR reaction comprising primer pair 23, icd (606) extension primer is binding to the PCR product obtained from the PCR reaction comprising primer pair 24, purA extension primer is binding to the PCR product obtained from the PCR reaction comprising primer pair 25, lexA extension primer is binding to the PCR product obtained from the PCR reaction comprising primer pair 26 and rpoH extension primer is binding to the PCR product obtained from the PCR reaction comprising primer pair 27.

All primers indicated under points i. to o. and (VI) to (XIII) above may comprise a sequence having at least 80%, more preferably at least 85%, more preferably at least 90%, more preferably at least 93% sequence identity with the sequences of the indicated SEQ ID NOs.

In embodiments of the present invention, the extension primers do not only comprise the sequences indicated in table 9 or variants having a sequence identity of at least 80%, more preferably at least 85% more preferably at least 90%, more preferably at least 93% therewith but even consist of these sequences. However, the present inventors found that it may be advantageous if non-complementary bases are added at the 5′-end of the primer so that each primer has a detectable different mass. Preferably, in addition to the sequences described in table 8 or to variants thereof as indicated above, the extension primers comprise at least 1, more preferably at least 2 non-complementary bases at the 5′-end. However, the number of non-complementary bases at the 5′-end should be limited. Preferably, the extension primers comprise at most 10, more preferably at most 5, more preferably at most 4 non-complementary bases at the 5′-end. It was found that non-complementary bases at the 5′-end are particularly preferred for gapA (420) extension primers, gapA (456) extension primers, infB (1608) extension primers, mdh (633) extension primers, phoE (732) extension primers, rpoB (1819) extension primers and gyrB extension primers.

Sequences that are comprised in most preferred extension primers of the present invention comprising non-complementary bases at the 5′-end are summarized in the following table 10. However, variants having a sequence identity of at least 80%, more preferably at least 85% more preferably at least 90%, more preferably at least 93% with the sequences indicated in table 10 are also preferable sequences of the present invention. The letter “I” in the sequences in table 10 indicates inosine.

TABLE 10

| | SEQ | |
|---|---|---|
| | ID | Comprised |
| Primer name | NO: | sequence (5′-3′) |
| gapA (420) extension long | 27 | GAGGGGAAACGATGTCCTGGCC |
| gapA (456) extension long | 30 | CCGCITCCTGCACCAC |
| infB (1608) extension | 33 | GTGTGCGGCGAGAGCCAGTT |
| long | | |
| mdh (633) extension long | 36 | AAGTATTCAIAAIGCCGGTAC |
| phoE (732) extension long | 39 | GGCTGATCGGGGTCAT |
| rpoB (1819) extension | 45 | GGCICCGTATCGTAAAGTGACC |
| long | | |
| gyrB extension long | 112 | CCCCCACGGTGTACCGCAGGC |

Preferably, at least one, more preferably at least two, more preferably at least three, more preferably at least four, more preferably at least five, more preferably at least six, more preferably all seven of the following extension primers are allowed to bind to the PCR products obtained from the PCR reactions of step A) in addition to the extension primers of the antibiotic resistance markers as indicated above:
i. gapA (420) extension primer comprising a sequence of SEQ ID NO:27 or a sequence having a sequence identity of at least 80%, more preferably at least 85% more preferably at least 90%, more preferably at least 93% with SEQ ID NO:27,
j. gapA (456) extension primer comprising a sequence of SEQ ID NO:30 or a sequence having a sequence identity of at least 80%, more preferably at least 85% more preferably at least 90%, more preferably at least 93% with SEQ ID NO:30,
k. infB (1608) extension primer comprising a sequence of SEQ ID NO:33 or a sequence having a sequence identity of at least 80%, more preferably at least 85% more preferably at least 90%, more preferably at least 93% with SEQ ID NO:33,
l. mdh (633) extension primer comprising a sequence of SEQ ID NO:36 or a sequence having a sequence identity of at least 80%, more preferably at least 85% more preferably at least 90%, more preferably at least 93% with SEQ ID NO:36,
m. phoE (732) extension primer comprising a sequence of SEQ ID NO:39 or a sequence having a sequence identity of at least 80%, more preferably at least 85% more preferably at least 90%, more preferably at least 93% with SEQ ID NO:39,
n. phoE (867) extension primer comprising a sequence of SEQ ID NO:42 or a sequence having a sequence identity of at least 80%, more preferably at least 85% more preferably at least 90%, more preferably at least 93% with SEQ ID NO:42,
o. rpoB (1819) extension primer comprising a sequence of SEQ ID NO:45 or a sequence having a sequence identity of at least 80%, more preferably at least 85% more preferably at least 90%, more preferably at least 93% with SEQ ID NO:45.

Preferably, gapA (420) extension primer and gapA (456) extension primer are binding to the PCR product obtained from the PCR reaction comprising primer pair 14, infB (1608) extension primer is binding to the PCR product obtained from the PCR reaction comprising primer pair 15, mdh (633) extension primer is binding to the PCR product obtained from the PCR reaction comprising primer pair 16, phoE (732) extension primer is binding to the PCR product obtained from the PCR reaction comprising primer pair 17, phoE (867) extension primer is binding to the PCR product obtained from the PCR reaction comprising primer pair 18 and rpoB (1819) extension primer is binding to the PCR product obtained from the PCR reaction comprising primer pair 19.

Additionally or alternatively to the extension primers described under points i. to o. above, preferably at least one, more preferably at least two, more preferably at least three, more preferably at least four, more preferably at least five, more preferably at least six, more preferably at least seven, more preferably all eight of the following extension primers are allowed to bind to the PCR products obtained from the PCR reactions of step A) in addition to the extension primers of the antibiotic resistance markers as indicated above:
(VI) gyrB extension primer comprising a sequence of SEQ ID NO:112 or a sequence having a sequence identity of at least 80%, more preferably at least 85% more preferably at least 90%, more preferably at least 93% with SEQ ID NO:112,
(VII) adk (348) extension primer comprising a sequence of SEQ ID NO:115 or a sequence having a sequence identity of at least 80%, more preferably at least 85% more preferably at least 90%, more preferably at least 93% with SEQ ID NO:115,
(VIII) adk (456) extension primer comprising a sequence of SEQ ID NO:118 or a sequence having a sequence identity of at least 80%, more preferably at least 85% more preferably at least 90%, more preferably at least 93% with SEQ ID NO:118,
(IX) icd (243) extension primer comprising a sequence of SEQ ID NO:121 or a sequence having a sequence identity of at least 80%, more preferably at least 85% more preferably at least 90%, more preferably at least 93% with SEQ ID NO:121,
(X) icd (606) extension primer comprising a sequence of SEQ ID NO:124 or a sequence having a sequence identity of at least 80%, more preferably at least 85% more preferably at least 90%, more preferably at least 93% with SEQ ID NO:124,
(XI) purA extension primer comprising a sequence of SEQ ID NO:127 or a sequence having a sequence identity of at least 80%, more preferably at least 85% more preferably at least 90%, more preferably at least 93% with SEQ ID NO:127,
(XII) lexA extension primer comprising a sequence of SEQ ID NO:130 or a sequence having a sequence identity of at least 80%, more preferably at least 85% more preferably at least 90%, more preferably at least 93% with SEQ ID NO:130,
(XIII) rpoH extension primer comprising a sequence of SEQ ID NO:133 or a sequence having a sequence identity of at least 80%, more preferably at least 85% more preferably at least 90%, more preferably at least 93% with SEQ ID NO:133.

Preferably, gyrB extension primer is binding to the PCR product obtained from the PCR reaction comprising primer pair 20, adk (348) extension primer is binding to the PCR product obtained from the PCR reaction comprising primer pair 21, adk (456) extension primer is binding to the PCR product obtained from the PCR reaction comprising primer pair 22, icd (243) extension primer is binding to the PCR product obtained from the PCR reaction comprising primer pair 23, icd (606) extension primer is binding to the PCR product obtained from the PCR reaction comprising primer pair 24, purA extension primer is binding to the PCR product obtained from the PCR reaction comprising primer pair 25, lexA extension primer is binding to the PCR product obtained from the PCR reaction comprising primer pair 26 and rpoH extension primer is binding to the PCR product obtained from the PCR reaction comprising primer pair 27.

All primers indicated under points i. to o. and (VI) to (XIII) above may comprise a sequence having at least 80%, more preferably at least 85%, more preferably at least 90%, more preferably at least 93% sequence identity with the sequences of the indicated SEQ ID NOs.

Preferably, the extension primers indicated in table 10 do not only comprise the sequences indicated in table 10 or variants having a sequence identity of at least 80%, more preferably at least 85% more preferably at least 90%, more preferably at least 93% therewith but even consist of these sequences.

In step C) of the methods of the present invention, the extension primers described above are preferably elongated at the 3′-end by at least one nucleotide, preferably by exactly one nucleotide. Preferably, the elongation of all extension primers is performed in a single tube, more preferably in the same tube, in which the PCR reactions have been performed.

In step D) of the methods of the present invention, the at least one nucleotide, preferably exactly one nucleotide that has been added to the extension primer is determined. Preferably, the nucleotide is determined by mass spectrometry. Thus, the masses of the elongated extension primers are preferably determined by mass spectrometry. This enables identification of the nucleotide that has been added to the extension primer by comparison of the added mass with the known masses of the different nucleotides.

In step E) of the methods of the present invention, information on the phylogenetic origin is preferably determined based on a comparison of the pattern of the determined nucleotides with respective predetermined patterns of bacterial strain, which phylogenetic origin is known. In particular, the present inventors have found that determination of information on the phylogenetic origin of Klebsiella pneumoniae strains by the method of the present invention can be further improved if additional information on phylogenetic origin is generated from PCR reactions comprising at least one of primer pairs 14 to 19 in addition to primer pairs 1 to 8 and to the optional primer pairs 9 to 13 described above. Furthermore, the present inventors have found that determination of information on the phylogenetic origin of Escherichia coli strains by the method of the present invention can be further improved if additional information on phylogenetic origin is generated from PCR reactions comprising at least one of primer pairs 20 to 27 in addition to primer pairs 1 to 8 and to the optional primer pairs 9 to 13 described above.

Preferably, the primers of the present invention have length of at most 100 nucleotides, more preferably at most 50 nucleotides, more preferably at most 40 nucleotides.

In the methods of the present invention, the nucleotide being present at certain position may be determined by sequencing methods such as Sanger sequencing (dideoxy chain-termination method), Maxam-Gilbert sequencing, shotgun sequencing and next-generation sequencing methods such as pyrosequencing. However, the present inventors found that specifically satisfying results with regard to costs as well as specificity and sensitivity of detection are obtained when the nucleotides being present at certain positions are determined by a method that comprises a step of detection by mass spectrometry. A preferred mass spectrometry based detection method is MALDI-TOF (Matrix-Assisted Laser Desorption/lonization-Time Of Flight). In a preferred embodiment, determination of nucleotides involves PCR based DNA amplification and subsequent detection of the nucleotide by mass spectrometry.

In a particular preferred embodiment, determination of the nucleotides involves a process, which will in the following be termed “MassArray”. In such MassArray, relevant regions of bacterial DNA containing SNPs of interest are amplified by PCR in a first step. Subsequently, an extension primer binding to the DNA of interest just downstream of the nucleotide to be determined is elongated by at least one nucleotide at its 3′-end. Such elongated extension primer is then analyzed by mass spectrometry in order to determine the nucleotide that has been added to the primer and which is complementary to the nucleotide of interest, thus allowing determination of the nucleotide of interest.

In preferred embodiments, the extension primer is elongated by exactly one nucleotide. This is preferably achieved by use of chain terminating nucleotides, thus by nucleotides that prevent further elongation of the extension primer once they have been added to the 3′-end of the primer. Chain terminating nucleotides of the present invention are for example dideoxynucleotides (ddNTPs). However, the mass differences between different ddNTPs, in particular between ddATP and ddTTP, are comparably small, which complicates distinguishing by mass spectrometry. Therefore, particularly preferred chain terminating nucleotides of the present invention are acyclonucleotides (acyNTPs). Acyclonucleotides are chain terminators that lack a ribose 3′-OH required for further extension. Once an acyclonucleotide is incorporated by a DNA polymerase, the substrate can no longer be further extended. Acyclonucleotides are especially useful in applications with archaeon DNA polymerases, more preferably with Therminator DNA Polymerase. Therminator DNA polymerase is an engineered enzyme with an increased capacity to incorporate analogs with altered sugars, such as ribonucleotides, dideoxynucleotides, 2′ deoxynucleotides and especially acyclo-base analogs.

The present inventors found that MassArray is a preferred technology for determination of the nucleotide of interest, particularly in combination with multiplex PCR, because it is rapid and robust and allows for the detection of many nucleotides of interest at reasonable costs. In particular, the present inventors found that the extension primers described above are specifically suitable for detection of the nucleotides of interest by MassArray.

The methods of the present invention may comprise determination of nucleotides present at further gene positions. Determination of more nucleotides renders the method more laborious and complex. However, on the other hand, sensitivity of determination of information on phylogenetic origin and/or antibiotic resistances may be further enhanced by determination of more nucleotides.

As found by the present inventors, the pattern of determined nucleotides of the present invention is very specific for individual bacterial isolates. Thus, by the methods of the present invention information on the phylogenetic origin of a bacterial isolate can be determined in addition to the antibiotic resistances by comparison of the pattern of the determined nucleotides with respective predetermined patterns of bacterial strains, of which phylogenetic origin and antibiotic resistances are known.

The present invention also comprises a kit for performing the method of the invention, wherein the kit comprises
(i) sets of PCR primers for amplification of bacterial DNA regions that include the nucleotides to be determined, and
(ii) extension primers for determining which nucleotide is present
   a. at position 814 in the KPC-2 coding sequence (SEQ ID NO:87),
   b. at position 82 in the NDM-1 coding sequence (SEQ ID NO:88),
   c. at position 104 in the OXA-9 coding sequence (SEQ ID NO:89),
   d. at position 186 in the OXA-48 coding sequence (SEQ ID NO:90),
   e. at position 556 in the CTX-M-9 coding sequence (SEQ ID NO:91),
   f. at position 453 in the CTX-M-15 coding sequence (SEQ ID NO:92),
   g. at position 223 in the AAC(6)-lb-cr coding sequence (SEQ ID NO:93), and
   h. at position 454 in the AAC(6)-lb-cr coding sequence (SEQ ID NO:93),
   or at analogous positions in variants or homologous coding sequences having at least 80% sequence identity with the indicated coding sequences.

Preferably, the kit additionally comprises
(ii) extension primers for determining which nucleotide is present
   (I) at position 408 in the OXA-1 coding sequence (SEQ ID NO:163),
   (II) at position 585 in the OXA-2 coding sequence (SEQ ID NO:164),
   (III) at position 228 in the TEM-1 coding sequence (SEQ ID NO:165),
   (IV) at position 230 in the APHA3-la coding sequence (SEQ ID NO:166), and/or
   (V) at position 63 in the AAC(3′)-IIae coding sequence (SEQ ID NO:167),
   or at analogous positions in variants or homologous coding sequences having at least 80% sequence identity with the indicated coding sequences.

Preferably, the kit comprises at least one, more preferably at least two, more preferably at least three, more preferably at least four, more preferably all five of extension primers (I) to (V) as indicated above.

Preferably, the variants or homologous coding sequences have a sequence identity with the coding sequences as depicted in SEQ ID NOs: 87-93 of at least 90%, more preferably of at least 95%, more preferably of at least 98%, even more preferably of at least 99%. Preferably, the variants or homologous coding sequences have a sequence identity with the coding sequences as depicted in SEQ ID NOs: 163-167 of at least 90%, more preferably of at least 95%, more preferably of at least 98%, even more preferably of at least 99%.

The present invention also relates to a kit comprising
(i) the following amplification primers:
   a. A forward primer comprising a sequence of SEQ ID NO:46 or a sequence having at least 80%, preferably at least 85%, more preferably at least 90%, more preferably at least 94% sequence identity therewith, and a reverse primer comprising a sequence of SEQ ID NO:47 or a sequence having at least 80%, preferably at least 85%, more preferably at least 90%, more preferably at least 94% sequence identity therewith,
   b. A forward primer comprising a sequence of SEQ ID NO:48 or a sequence having at least 80%, preferably at least 85%, more preferably at least 90%, more preferably at least 94% sequence identity therewith, and a reverse primer comprising a sequence of SEQ ID NO:49 or a sequence having at least 80%, preferably at least 85%, more preferably at least 90%, more preferably at least 94% sequence identity therewith,
   c. A forward primer comprising a sequence of SEQ ID NO:51 or a sequence having at least 80%, preferably at least 85%, more preferably at least 90%, more preferably at least 94% sequence identity therewith, and a reverse primer comprising a sequence of SEQ ID NO:52 or a sequence having at least 80%, preferably at least 85%, more preferably at least 90%, more preferably at least 94% sequence identity therewith,
   d. A forward primer comprising a sequence of SEQ ID NO:54 or a sequence having at least 80%, preferably at least 85%, more preferably at least 90%, more preferably at least 94% sequence identity therewith, and a reverse primer comprising a sequence of SEQ ID NO:55 or a sequence having at least 80%, preferably at least 85%, more preferably at least 90%, more preferably at least 94% sequence identity therewith,
   e. A forward primer comprising a sequence of SEQ ID NO:57 or a sequence having at least 80%, preferably at least 85%, more preferably at least 90%, more preferably at least 94% sequence identity therewith, and a reverse primer comprising a sequence of SEQ ID NO:58 or a sequence having at least 80%, preferably at least 85%, more preferably at least 90%, more preferably at least 94% sequence identity therewith,
   f. A forward primer comprising a sequence of SEQ ID NO:60 or a sequence having at least 80%, preferably at least 85%, more preferably at least 90%, more preferably at least 94% sequence identity therewith, and a reverse primer comprising a sequence of SEQ ID NO:61 or a sequence having at least 80%, preferably at least 85%, more preferably at least 90%, more preferably at least 94% sequence identity therewith,
   g. A forward primer comprising a sequence of SEQ ID NO:62 or a sequence having at least 80%, preferably at least 85%, more preferably at least 90%, more preferably at least 94% sequence identity therewith, and a reverse primer comprising a sequence of SEQ ID NO:63 or a sequence having at least 80%, preferably at least 85%, more preferably at least 90%, more preferably at least 94% sequence identity therewith, and
   h. A forward primer comprising a sequence of SEQ ID NO:65 or a sequence having at least 80%, preferably at least 85%, more preferably at least 90%, more preferably at least 94% sequence identity therewith, and a reverse primer comprising a sequence of SEQ ID NO:66 or a sequence having at least 80%, preferably at least 85%, more preferably at least 90%, more preferably at least 94% sequence identity therewith.

Preferably, the kit additionally comprises at least one, more preferably at least two, more preferably at least three, more preferably at least four, more preferably all five of
(i) the following amplification primers:
   (I) A forward primer comprising a sequence of SEQ ID NO:134 or a sequence having at least 80%, preferably at least 85%, more preferably at least 90%, more preferably at least 94% sequence identity therewith, and a reverse primer comprising a sequence of SEQ ID NO:135 or a sequence having at least 80%, preferably at least 85%, more preferably at least 90%, more preferably at least 94% sequence identity therewith,
   (II) A forward primer comprising a sequence of SEQ ID NO:136 or a sequence having at least 80%, preferably at least 85%, more preferably at least 90%, more preferably at least 94% sequence identity therewith, and a reverse primer comprising a sequence of SEQ ID NO:137 or a sequence having at least 80%, preferably at least 85%, more preferably at least 90%, more preferably at least 94% sequence identity therewith,
   (III) A forward primer comprising a sequence of SEQ ID NO:138 or a sequence having at least 80%, preferably at least 85%, more preferably at least 90%, more preferably at least 94% sequence identity therewith, and a reverse primer comprising a sequence of SEQ ID NO:139 or a sequence having at least 80%, preferably at least 85%, more preferably at least 90%, more preferably at least 94% sequence identity therewith,
   (IV) A forward primer comprising a sequence of SEQ ID NO:141 or a sequence having at least 80%, preferably at least 85%, more preferably at least 90%, more preferably at least 94% sequence identity therewith, and a reverse primer comprising a sequence of SEQ ID NO:142 or a sequence having at least 80%, preferably at least 85%, more preferably at least 90%, more preferably at least 94% sequence identity therewith,
   (V) A forward primer comprising a sequence of SEQ ID NO:144 or a sequence having at least 80%, preferably at least 85%, more preferably at least 90%, more preferably at least 94% sequence identity therewith, and a reverse primer comprising a sequence of SEQ ID NO:145 or a sequence having at least 80%, preferably at least 85%, more preferably at least 90%, more preferably at least 94% sequence identity therewith.

All primers of the primer pairs indicated under points a. to h. and (I) to (V) above may comprise a sequence having at least 80%, more preferably at least 85%, more preferably at least 90%, more preferably at least 94% sequence identity with the sequences of the indicated SEQ ID NOs.

Preferably, the kit additionally comprises
(ii) the following extension primers:
   a. KPC-2 extension primer comprising a sequence of SEQ ID NO:3 or a sequence having a sequence identity of at least 80%, more preferably at least 85% more preferably at least 90%, more preferably at least 93% with SEQ ID NO:3,
   b. NDM-1 extension primer comprising a sequence of SEQ ID NO:50 or a sequence having a sequence identity of at least 80%, more preferably at least 85% more preferably at least 90%, more preferably at least 93% with SEQ ID NO:50,
   c. OXA-9 extension primer comprising a sequence of SEQ ID NO:53 or a sequence having a sequence identity of at least 80%, more preferably at least 85% more preferably at least 90%, more preferably at least 93% with SEQ ID NO:53,
   d. OXA-48 extension primer comprising a sequence of SEQ ID NO:56 or a sequence having a sequence identity of at least 80%, more preferably at least 85% more preferably at least 90%, more preferably at least 93% with SEQ ID NO:56,
   e. CTX-M-9 extension primer comprising a sequence of SEQ ID NO:59 or a sequence having a sequence identity of at least 80%, more preferably at least 85% more preferably at least 90%, more preferably at least 93% with SEQ ID NO:59,
   f. CTX-M-15 extension primer comprising a sequence of SEQ ID NO:18 or a sequence having a sequence identity of at least 80%, more preferably at least 85% more preferably at least 90%, more preferably at least 93% with SEQ ID NO:18,
   g. AAC(6′)-lb-cr (223) extension primer comprising a sequence of SEQ ID NO:64 or a sequence having a sequence identity of at least 80%, more preferably at least 85% more preferably at least 90%, more preferably at least 93% with SEQ ID NO:64, and
   h. AAC(6′)-lb-cr (454) extension primer comprising a sequence of SEQ ID NO:24 or a sequence having a sequence identity of at least 80%, more preferably at least 85% more preferably at least 90%, more preferably at least 93% with SEQ ID NO:24.

Preferably, the kit additionally comprises at least one, more preferably at least two, more preferably at least three, more preferably at least four, more preferably all five of
(ii) the following extension primers:
   (I) OXA-1 extension primer comprising a sequence of SEQ ID NO:97 or a sequence having a sequence identity of at least 80%, more preferably at least 85% more preferably at least 90%, more preferably at least 93% with SEQ ID NO:97,
   (II) OXA-2 extension primer comprising a sequence of SEQ ID NO:100 or a sequence having a sequence identity of at least 80%, more preferably at least 85% more preferably at least 90%, more preferably at least 93% with SEQ ID NO:100,
   (III) TEM-1 extension primer comprising a sequence of SEQ ID NO:140 or a sequence having a sequence identity of at least 80%, more preferably at least 85% more preferably at least 90%, more preferably at least 93% with SEQ ID NO:140,
   (IV) APHA3-la extension primer comprising a sequence of SEQ ID NO:143 or a sequence having a sequence identity of at least 80%, more preferably at least 85% more preferably at least 90%, more preferably at least 93% with SEQ ID NO:143,
   (V) AAC(3′)-IIae extension primer comprising a sequence of SEQ ID NO:109 or a sequence having a sequence identity of at least 80%, more preferably at least 85% more preferably at least 90%, more preferably at least 93% with SEQ ID NO:109.

All primers indicated under points a. to h. and (I) to (V) above may comprise a sequence having at least 80%, more preferably at least 85%, more preferably at least 90%, more preferably at least 93% sequence identity with the sequences of the indicated SEQ ID NOs.

In embodiments of the present invention, the amplification primers do not only comprise the sequences indicated above or variants having a sequence identity of at least 80%, more preferably at least 85% more preferably at least 90%, more preferably at least 94% therewith but even consist of these sequences. However, the present inventors found that the amplification is advantageously balanced if the amplification primers additionally comprise nucleotides at the 5′-end. Furthermore, this increases the masses of unused primers (out of the mass range on the spectra).

In embodiments of the present invention, the extension primers do not only comprise the sequences indicated above or variants having a sequence identity of at least 80%, more preferably at least 85% more preferably at least 90%, more preferably at least 93% therewith but even consist of these sequences. However, the present inventors found that it may be advantageous if non-complementary bases are added at the 5′-end of the primers so that each primer has a detectable different mass.

A particularly preferred kit of the present invention is a kit for performing the methods of the invention, wherein the kit comprises
(i) the following amplification primers:
   a. A forward primer comprising a sequence of SEQ ID NO:1 or a sequence having at least 80%, preferably at least 85%, more preferably at least 90%, more preferably at least 95% sequence identity therewith, and a reverse primer comprising a sequence of SEQ ID NO:2 or a sequence having at least 80%, preferably at least 85%, more preferably at least 90%, more preferably at least 95% sequence identity therewith,
   b. A forward primer comprising a sequence of SEQ ID NO:4 or a sequence having at least 80%, preferably at least 85%, more preferably at least 90%, more preferably at least 95% sequence identity therewith, and a reverse primer comprising a sequence of SEQ ID NO:5 or a sequence having at least 80%, preferably at least 85%, more preferably at least 90%, more preferably at least 95% sequence identity therewith,
   c. A forward primer comprising a sequence of SEQ ID NO:7 or a sequence having at least 80%, preferably at least 85%, more preferably at least 90%, more preferably at least 95% sequence identity therewith, and a reverse primer comprising a sequence of SEQ ID NO:8 or a sequence having at least 80%, preferably at least 85%, more preferably at least 90%, more preferably at least 95% sequence identity therewith,
   d. A forward primer comprising a sequence of SEQ ID NO:10 or a sequence having at least 80%, preferably at least 85%, more preferably at least 90%, more preferably at least 95% sequence identity therewith, and a reverse primer comprising a sequence of SEQ ID NO:11 or a sequence having at least 80%, preferably at least 85%, more preferably at least 90%, more preferably at least 95% sequence identity therewith,
   e. A forward primer comprising a sequence of SEQ ID NO:13 or a sequence having at least 80%, preferably at least 85%, more preferably at least 90%, more preferably at least 95% sequence identity therewith, and a reverse primer comprising a sequence of SEQ ID NO:14 or a sequence having at least 80%, preferably at least 85%, more preferably at least 90%, more preferably at least 95% sequence identity therewith,
   f. A forward primer comprising a sequence of SEQ ID NO:16 or a sequence having at least 80%, preferably at least 85%, more preferably at least 90%, more preferably at least 95% sequence identity therewith, and a reverse primer comprising a sequence of SEQ ID NO:17 or a sequence having at least 80%, preferably at least 85%, more preferably at least 90%, more preferably at least 95% sequence identity therewith,
   g. A forward primer comprising a sequence of SEQ ID NO:19 or a sequence having at least 80%, preferably at least 85%, more preferably at least 90%, more preferably at least 95% sequence identity therewith, and a reverse primer comprising a sequence of SEQ ID NO:20 or a sequence having at least 80%, preferably at least 85%, more preferably at least 90%, more preferably at least 95% sequence identity therewith, and
   h. A forward primer comprising a sequence of SEQ ID NO:22 or a sequence having at least 80%, preferably at least 85%, more preferably at least 90%, more preferably at least 95% sequence identity therewith, and a reverse primer comprising a sequence of SEQ ID NO:23 or a sequence having at least 80%, preferably at least 85%, more preferably at least 90%, more preferably at least 95% sequence identity therewith.

Preferably, the kit additionally comprises at least one, more preferably at least two, more preferably at least three, more preferably at least four, more preferably all five of
(i) the following amplification primers:
   (I) A forward primer comprising a sequence of SEQ ID NO:95 or a sequence having at least 80%, preferably at least 85%, more preferably at least 90%, more preferably at least 95% sequence identity therewith, and a reverse primer comprising a sequence of SEQ ID NO:96 or a sequence having at least 80%, preferably at least 85%, more preferably at least 90%, more preferably at least 95% sequence identity therewith,
   (II) A forward primer comprising a sequence of SEQ ID NO:98 or a sequence having at least 80%, preferably at least 85%, more preferably at least 90%, more preferably at least 95% sequence identity therewith, and a reverse primer comprising a sequence of SEQ ID NO:99 or a sequence having at least 80%, preferably at least 85%, more preferably at least 90%, more preferably at least 95% sequence identity therewith,
   (III) A forward primer comprising a sequence of SEQ ID NO:101 or a sequence having at least 80%, preferably at least 85%, more preferably at least 90%, more preferably at least 95% sequence identity therewith, and a reverse primer comprising a sequence of SEQ ID NO:102 or a sequence having at least 80%, preferably at least 85%, more preferably at least 90%, more preferably at least 95% sequence identity therewith,
   (IV) A forward primer comprising a sequence of SEQ ID NO:104 or a sequence having at least 80%, preferably at least 85%, more preferably at least 90%, more preferably at least 95% sequence identity therewith, and a reverse primer comprising a sequence of SEQ ID NO:105 or a sequence having at least 80%, preferably at least 85%, more preferably at least 90%, more preferably at least 95% sequence identity therewith,
   (V) A forward primer comprising a sequence of SEQ ID NO:107 or a sequence having at least 80%, preferably at least 85%, more preferably at least 90%, more preferably at least 95% sequence identity therewith, and a reverse primer comprising a sequence of SEQ ID NO:108 or a sequence having at least 80%, preferably at least 85%, more preferably at least 90%, more preferably at least 95% sequence identity therewith.

All primers of the primer pairs indicated under points a. to h. and (I) to (V) above may comprise a sequence having at least 80%, more preferably at least 85%, more preferably at least 90%, more preferably at least 95% sequence identity with the sequences of the indicated SEQ ID NOs.

Preferably, the kit additionally comprises
(ii) the following extension primers:
   a. KPC-2 extension primer comprising a sequence of SEQ ID NO:3 or a sequence having a sequence identity of at least 80%, more preferably at least 85% more preferably at least 90%, more preferably at least 93% with SEQ ID NO:3,
   b. NDM-1 extension primer comprising a sequence of SEQ ID NO:6 or a sequence having a sequence identity of at least 80%, more preferably at least 85% more preferably at least 90%, more preferably at least 93% with SEQ ID NO:6,
   c. OXA-9 extension primer comprising a sequence of SEQ ID NO:9 or a sequence having a sequence identity of at least 80%, more preferably at least 85% more preferably at least 90%, more preferably at least 93% with SEQ ID NO:9,
   d. OXA-48 extension primer comprising a sequence of SEQ ID NO:12 or a sequence having a sequence identity of at least 80%, more preferably at least 85% more preferably at least 90%, more preferably at least 93% with SEQ ID NO:12,
   e. CTX-M-9 extension primer comprising a sequence of SEQ ID NO:15 or a sequence having a sequence identity of at least 80%, more preferably at least 85% more preferably at least 90%, more preferably at least 93% with SEQ ID NO:15,
   f. CTX-M-15 extension primer comprising a sequence of SEQ ID NO:18 or a sequence having a sequence identity of at least 80%, more preferably at least 85% more preferably at least 90%, more preferably at least 93% with SEQ ID NO:18,
   g. AAC(6′)-lb-cr (223) extension primer comprising a sequence of SEQ ID NO:21 or a sequence having a sequence identity of at least 80%, more preferably at least 85% more preferably at least 90%, more preferably at least 93% with SEQ ID NO:21, and
   h. AAC(6′)-lb-cr (454) extension primer comprising a sequence of SEQ ID NO:24 or a sequence having a sequence identity of at least 80%, more preferably at least 85% more preferably at least 90%, more preferably at least 93% with SEQ ID NO:24.

Preferably, the kit additionally comprises at least one, more preferably at least two, more preferably at least three, more preferably at least four, more preferably all five of
(ii) the following extension primers:
   (I) OXA-1 extension primer comprising a sequence of SEQ ID NO:97 or a sequence having a sequence identity of at least 80%, more preferably at least 85% more preferably at least 90%, more preferably at least 93% with SEQ ID NO:97,
   (II) OXA-2 extension primer comprising a sequence of SEQ ID NO:100 or a sequence having a sequence identity of at least 80%, more preferably at least 85% more preferably at least 90%, more preferably at least 93% with SEQ ID NO:100,
   (III) TEM-1 extension primer comprising a sequence of SEQ ID NO:103 or a sequence having a sequence identity of at least 80%, more preferably at least 85% more preferably at least 90%, more preferably at least 93% with SEQ ID NO:103,
   (IV) APHA3-la extension primer comprising a sequence of SEQ ID NO:106 or a sequence having a sequence identity of at least 80%, more preferably at least 85% more preferably at least 90%, more preferably at least 93% with SEQ ID NO:106,
   (V) AAC(3′)-IIae extension primer comprising a sequence of SEQ ID NO:109 or a sequence having a sequence identity of at least 80%, more preferably at least 85% more preferably at least 90%, more preferably at least 93% with SEQ ID NO:109.

All primers indicated under points a. to h. and (I) to (V) above may comprise a sequence having at least 80%, more preferably at least 85%, more preferably at least 90%, more preferably at least 93% sequence identity with the sequences of the indicated SEQ ID NOs.

Preferably, the amplifying primers do not only comprise the sequences indicated above or variants having a sequence identity of at least 80%, more preferably at least 85% more preferably at least 90%, more preferably at least 95% therewith but even consist of these sequences.

Preferably, the extension primers indicated above do not only comprise the sequences indicated above or variants having a sequence identity of at least 80%, more preferably at least 85% more preferably at least 90%, more preferably at least 93% therewith but even consist of these sequences.

In preferred embodiments, the kits of the present invention comprise further primers. Particularly preferably, the kits of the invention additionally comprise
(i) at least one, more preferably at least two, more preferably at least three, more preferably at least four, more preferably at least five, more preferably all six of the following amplification primer pairs:
   i. Primer pair 14 consisting of a forward primer comprising a sequence of SEQ ID NO:67 or a sequence having at least 80%, preferably at least 85%, more preferably at least 90%, more preferably at least 94% sequence identity therewith, and a reverse primer comprising a sequence of SEQ ID NO:68 or a sequence having at least 80%, preferably at least 85%, more preferably at least 90%, more preferably at least 94% sequence identity therewith,
   j. Primer pair 15 consisting of a forward primer comprising a sequence of SEQ ID NO:73 or a sequence having at least 80%, preferably at least 85%, more preferably at least 90%, more preferably at least 94% sequence identity therewith, and a reverse primer comprising a sequence of SEQ ID NO:74 or a sequence having at least 80%, preferably at least 85%, more preferably at least 90%, more preferably at least 94% sequence identity therewith,
   k. Primer pair 16 consisting of a forward primer comprising a sequence of SEQ ID NO:76 or a sequence having at least 80%, preferably at least 85%, more preferably at least 90%, more preferably at least 94% sequence identity therewith, and a reverse primer comprising a sequence of SEQ ID NO:77 or a sequence having at least 80%, preferably at least 85%, more preferably at least 90%, more preferably at least 94% sequence identity therewith,
   l. Primer pair 17 consisting of a forward primer comprising a sequence of SEQ ID NO:79 or a sequence having at least 80%, preferably at least 85%, more preferably at least 90%, more preferably at least 94% sequence identity therewith, and a reverse primer comprising a sequence of SEQ ID NO:80 or a sequence having at least 80%, preferably at least 85%, more preferably at least 90%, more preferably at least 94% sequence identity therewith,
   m. Primer pair 18 consisting of a forward primer comprising a sequence of SEQ ID NO:82 or a sequence having at least 80%, preferably at least 85%, more preferably at least 90%, more preferably at least 94% sequence identity therewith, and a reverse primer comprising a sequence of SEQ ID NO:83 or a sequence having at least 80%, preferably at least 85%, more preferably at least 90%, more preferably at least 94% sequence identity therewith, and
   n. Primer pair 19 consisting of a forward primer comprising a sequence of SEQ ID NO:84 or a sequence having at least 80%, preferably at least 85%, more preferably at least 90%, more preferably at least 94% sequence identity therewith, and a reverse primer comprising a sequence of SEQ ID NO:85 or a sequence having at least 80%, preferably at least 85%, more preferably at least 90%, more preferably at least 94% sequence identity therewith.

Additionally or alternatively to primer pairs 14 to 19 described above, the kits preferably comprise
(i) at least one, more preferably at least two, more preferably at least three, more preferably at least four, more preferably at least five, more preferably at least six, more preferably at least seven, more preferably all eight of the following amplification primer pairs:
   (VI) Primer pair 20 consisting of a forward primer comprising a sequence of SEQ ID NO:146 or a sequence having at least 80%, preferably at least 85%, more preferably at least 90%, more preferably at least 94% sequence identity therewith, and a reverse primer comprising a sequence of SEQ ID NO:147 or a sequence having at least 80%, preferably at least 85%, more preferably at least 90%, more preferably at least 94% sequence identity therewith,
   (VII) Primer pair 21 consisting of a forward primer comprising a sequence of SEQ ID NO:149 or a sequence having at least 80%, preferably at least 85%, more preferably at least 90%, more preferably at least 94% sequence identity therewith, and a reverse primer comprising a sequence of SEQ ID NO:150 or a sequence having at least 80%, preferably at least 85%, more preferably at least 90%, more preferably at least 94% sequence identity therewith,
   (VIII) Primer pair 22 consisting of a forward primer comprising a sequence of SEQ ID NO:151 or a sequence having at least 80%, preferably at least 85%, more preferably at least 90%, more preferably at least 94% sequence identity therewith, and a reverse primer comprising a sequence of SEQ ID NO:152 or a sequence having at least 80%, preferably at least 85%, more preferably at least 90%, more preferably at least 94% sequence identity therewith,
   (IX) Primer pair 23 consisting of a forward primer comprising a sequence of SEQ ID NO:153 or a sequence having at least 80%, preferably at least 85%, more preferably at least 90%, more preferably at least 94% sequence identity therewith, and a reverse primer comprising a sequence of SEQ ID NO:154 or a sequence having at least 80%, preferably at least 85%, more preferably at least 90%, more preferably at least 94% sequence identity therewith,
   (X) Primer pair 24 consisting of a forward primer comprising a sequence of SEQ ID NO:155 or a sequence having at least 80%, preferably at least 85%, more preferably at least 90%, more preferably at least 94% sequence identity therewith, and a reverse primer comprising a sequence of SEQ ID NO:156 or a sequence having at least 80%, preferably at least 85%, more preferably at least 90%, more preferably at least 94% sequence identity therewith,
   (XI) Primer pair 25 consisting of a forward primer comprising a sequence of SEQ ID NO:157 or a sequence having at least 80%, preferably at least 85%, more preferably at least 90%, more preferably at least 94% sequence identity therewith, and a reverse primer comprising a sequence of SEQ ID NO:158 or a sequence having at least 80%, preferably at least 85%, more preferably at least 90%, more preferably at least 94% sequence identity therewith,
   (XII) Primer pair 26 consisting of a forward primer comprising a sequence of SEQ ID NO:159 or a sequence having at least 80%, preferably at least 85%, more preferably at least 90%, more preferably at least 94% sequence identity therewith, and a reverse primer comprising a sequence of SEQ ID NO:160 or a sequence having at least 80%, preferably at least 85%, more preferably at least 90%, more preferably at least 94% sequence identity therewith,
   (XIII) Primer pair 27 consisting of a forward primer comprising a sequence of SEQ ID NO:161 or a sequence having at least 80%, preferably at least 85%, more preferably at least 90%, more preferably at least 94% sequence identity therewith, and a reverse primer comprising a sequence of SEQ ID NO:162 or a sequence having at least 80%, preferably at least 85%, more preferably at least 90%, more preferably at least 94% sequence identity therewith.

All primers of the primer pairs indicated under points i. to n. and (VI) to (XIII) above may comprise a sequence having at least 80%, more preferably at least 85%, more preferably at least 90%, more preferably at least 94% sequence identity with the sequences of the indicated SEQ ID NOs.

In embodiments of the present invention, the forward and reverse primers of primer pairs 14 to 27 do not only comprise the sequences of SEQ ID NOs: 67, 68, 73, 74, 76, 77, 79, 80, 82-85, 146, 147, 149-162 as indicated above or variants having a sequence identity of at least 80%, more preferably at least 85% more preferably at least 90%, more preferably at least 94% therewith but even consist of these sequences. However, the present inventors found that the amplification is advantageously balanced if the forward and reverse primers additionally comprise nucleotides at the 5′-end. Furthermore, this increases the masses of unused primers (out of the mass range on the spectra).

Therefore, a particularly preferred kit of the present invention comprises
(i) at least one, more preferably at least two, more preferably at least three, more preferably at least four, more preferably at least five, more preferably all six of the following primer pairs:
   i. Primer pair 14 consisting of a forward primer comprising a sequence of SEQ ID NO:25 or a sequence having at least 80%, preferably at least 85%, more preferably at least 90%, more preferably at least 95% sequence identity therewith, and a reverse primer comprising a sequence of SEQ ID NO:26 or a sequence having at least 80%, preferably at least 85%, more preferably at least 90%, more preferably at least 95% sequence identity therewith,
   j. Primer pair 15 consisting of a forward primer comprising a sequence of SEQ ID NO:31 or a sequence having at least 80%, preferably at least 85%, more preferably at least 90%, more preferably at least 95% sequence identity therewith, and a reverse primer comprising a sequence of SEQ ID NO:32 or a sequence having at least 80%, preferably at least 85%, more preferably at least 90%, more preferably at least 95% sequence identity therewith,
   k. Primer pair 16 consisting of a forward primer comprising a sequence of SEQ ID NO:34 or a sequence having at least 80%, preferably at least 85%, more preferably at least 90%, more preferably at least 95% sequence identity therewith, and a reverse primer comprising a sequence of SEQ ID NO:35 or a sequence having at least 80%, preferably at least 85%, more preferably at least 90%, more preferably at least 95% sequence identity therewith,
   l. Primer pair 17 consisting of a forward primer comprising a sequence of SEQ ID NO:37 or a sequence having at least 80%, preferably at least 85%, more preferably at least 90%, more preferably at least 95% sequence identity therewith, and a reverse primer comprising a sequence of SEQ ID NO:38 or a sequence having at least 80%, preferably at least 85%, more preferably at least 90%, more preferably at least 95% sequence identity therewith,
   m. Primer pair 18 consisting of a forward primer comprising a sequence of SEQ ID NO:40 or a sequence having at least 80%, preferably at least 85%, more preferably at least 90%, more preferably at least 95% sequence identity therewith, and a reverse primer comprising a sequence of SEQ ID NO:41 or a sequence having at least 80%, preferably at least 85%, more preferably at least 90%, more preferably at least 95% sequence identity therewith, and
   n. Primer pair 19 consisting of a forward primer comprising a sequence of SEQ ID NO:43 or a sequence having at least 80%, preferably at least 85%, more preferably at least 90%, more preferably at least 95% sequence identity therewith, and a reverse primer comprising a sequence of SEQ ID NO:44 or a sequence having at least 80%, preferably at least 85%, more preferably at least 90%, more preferably at least 95% sequence identity therewith.

Additionally or alternatively to primer pairs 14 to 19 described above, the kits preferably comprise
(i) at least one, more preferably at least two, more preferably at least three, more preferably at least four, more preferably at least five, more preferably at least six, more preferably at least seven, more preferably all eight of the following amplification primer pairs:
   (VI) Primer pair 20 consisting of a forward primer comprising a sequence of SEQ ID NO:110 or a sequence having at least 80%, preferably at least 85%, more preferably at least 90%, more preferably at least 95% sequence identity therewith, and a reverse primer comprising a sequence of SEQ ID NO:111 or a sequence having at least 80%, preferably at least 85%, more preferably at least 90%, more preferably at least 95% sequence identity therewith,
   (VII) Primer pair 21 consisting of a forward primer comprising a sequence of SEQ ID NO:113 or a sequence having at least 80%, preferably at least 85%, more preferably at least 90%, more preferably at least 95% sequence identity therewith, and a reverse primer comprising a sequence of SEQ ID NO:114 or a sequence having at least 80%, preferably at least 85%, more preferably at least 90%, more preferably at least 95% sequence identity therewith,
   (VIII) Primer pair 22 consisting of a forward primer comprising a sequence of SEQ ID NO:116 or a sequence having at least 80%, preferably at least 85%, more preferably at least 90%, more preferably at least 95% sequence identity therewith, and a reverse primer comprising a sequence of SEQ ID NO:117 or a sequence having at least 80%, preferably at least 85%, more preferably at least 90%, more preferably at least 95% sequence identity therewith,
   (IX) Primer pair 23 consisting of a forward primer comprising a sequence of SEQ ID NO:119 or a sequence having at least 80%, preferably at least 85%, more preferably at least 90%, more preferably at least 95% sequence identity therewith, and a reverse primer comprising a sequence of SEQ ID NO:120 or a sequence having at least 80%, preferably at least 85%, more preferably at least 90%, more preferably at least 95% sequence identity therewith,
   (X) Primer pair 24 consisting of a forward primer comprising a sequence of SEQ ID NO:122 or a sequence having at least 80%, preferably at least 85%, more preferably at least 90%, more preferably at least 95% sequence identity therewith, and a reverse primer comprising a sequence of SEQ ID NO:123 or a sequence having at least 80%, preferably at least 85%, more preferably at least 90%, more preferably at least 95% sequence identity therewith,
   (XI) Primer pair 25 consisting of a forward primer comprising a sequence of SEQ ID NO:125 or a sequence having at least 80%, preferably at least 85%, more preferably at least 90%, more preferably at least 95% sequence identity therewith, and a reverse primer comprising a sequence of SEQ ID NO:126 or a sequence having at least 80%, preferably at least 85%, more preferably at least 90%, more preferably at least 95% sequence identity therewith,
   (XII) Primer pair 26 consisting of a forward primer comprising a sequence of SEQ ID NO:128 or a sequence having at least 80%, preferably at least 85%, more preferably at least 90%, more preferably at least 95% sequence identity therewith, and a reverse primer comprising a sequence of SEQ ID NO:129 or a sequence having at least 80%, preferably at least 85%, more preferably at least 90%, more preferably at least 95% sequence identity therewith,
   (XIII) Primer pair 27 consisting of a forward primer comprising a sequence of SEQ ID NO:131 or a sequence having at least 80%, preferably at least 85%, more preferably at least 90%, more preferably at least 95% sequence identity therewith, and a reverse primer comprising a sequence of SEQ ID NO:132 or a sequence having at least 80%, preferably at least 85%, more preferably at least 90%, more preferably at least 95% sequence identity therewith.

All primers of the primer pairs indicated under points i. to n. (VI) to (XIII) above may comprise a sequence having at least 80%, more preferably at least 85%, more preferably at least 90%, more preferably at least 95% sequence identity with the sequences of the indicated SEQ ID NOs.

In preferred embodiments of the present invention, the forward and reverse primers do not only comprise the sequences of SEQ ID NOs: 25, 26, 31, 32, 34, 35, 37, 38, 40, 41, 43, 44, 110, 111, 113, 114, 116, 117, 119, 120, 122, 123, 125, 126, 128, 129, 131, 132 as indicated above or variants having a sequence identity of at least 80%, more preferably at least 85% more preferably at least 90%, more preferably at least 95% therewith but even consist of these sequences.

Preferably, the kits of the invention further comprise extension primers corresponding to primer pairs 14 to 19, wherein both gapA (420) and gapA (456) extension primer correspond to primer pair 14. Preferably, the kits comprise
(ii) at least one, more preferably at least two, more preferably at least three, more preferably at least four, more preferably at least five, more preferably at least six and even more preferably all seven of the following extension primers:
   i. gapA (420) extension primer comprising a sequence of SEQ ID NO:69 or a sequence having a sequence identity of at least 80%, more preferably at least 85% more preferably at least 90%, more preferably at least 93% with SEQ ID NO:69,
   j. gapA (456) extension primer comprising a sequence of SEQ ID NO:72 or a sequence having a sequence identity of at least 80%, more preferably at least 85% more preferably at least 90%, more preferably at least 93% with SEQ ID NO:72,
   k. infB (1608) extension primer comprising a sequence of SEQ ID NO:75 or a sequence having a sequence identity of at least 80%, more preferably at least 85% more preferably at least 90%, more preferably at least 93% with SEQ ID NO:75,
   l. mdh (633) extension primer comprising a sequence of SEQ ID NO:78 or a sequence having a sequence identity of at least 80%, more preferably at least 85% more preferably at least 90%, more preferably at least 93% with SEQ ID NO:78,
   m. phoE (732) extension primer comprising a sequence of SEQ ID NO:81 or a sequence having a sequence identity of at least 80%, more preferably at least 85% more preferably at least 90%, more preferably at least 93% with SEQ ID NO:81,
   n. phoE (867) extension primer comprising a sequence of SEQ ID NO:42 or a sequence having a sequence identity of at least 80%, more preferably at least 85% more preferably at least 90%, more preferably at least 93% with SEQ ID NO:42,
   o. rpoB (1819) extension primer comprising a sequence of SEQ ID NO:86 or a sequence having a sequence identity of at least 80%, more preferably at least 85% more preferably at least 90%, more preferably at least 93% with SEQ ID NO:86.

Additionally or alternatively to extension primers i. to o. indicated above, the kits of the invention preferably further comprise extension primers corresponding to primer pairs 20 to 27. Preferably, the kits comprise
(ii) at least one, more preferably at least two, more preferably at least three, more preferably at least four, more preferably at least five, more preferably at least six, more preferably at least seven, more preferably all eight of the following extension primers:
   (VI) gyrB extension primer comprising a sequence of SEQ ID NO:148 or a sequence having a sequence identity of at least 80%, more preferably at least 85% more preferably at least 90%, more preferably at least 93% with SEQ ID NO:148,
   (VII) adk (348) extension primer comprising a sequence of SEQ ID NO:115 or a sequence having a sequence identity of at least 80%, more preferably at least 85% more preferably at least 90%, more preferably at least 93% with SEQ ID NO:115,
   (VIII) adk (456) extension primer comprising a sequence of SEQ ID NO:118 or a sequence having a sequence identity of at least 80%, more preferably at least 85% more preferably at least 90%, more preferably at least 93% with SEQ ID NO:118,
   (IX) icd (243) extension primer comprising a sequence of SEQ ID NO:121 or a sequence having a sequence identity of at least 80%, more preferably at least 85% more preferably at least 90%, more preferably at least 93% with SEQ ID NO:121,
   (X) icd (606) extension primer comprising a sequence of SEQ ID NO:124 or a sequence having a sequence identity of at least 80%, more preferably at least 85% more preferably at least 90%, more preferably at least 93% with SEQ ID NO:124,
   (XI) purA extension primer comprising a sequence of SEQ ID NO:127 or a sequence having a sequence identity of at least 80%, more preferably at least 85% more preferably at least 90%, more preferably at least 93% with SEQ ID NO:127,
   (XII) lexA extension primer comprising a sequence of SEQ ID NO:130 or a sequence having a sequence identity of at least 80%, more preferably at least 85% more preferably at least 90%, more preferably at least 93% with SEQ ID NO:130,
   (XIII) rpoH extension primer comprising a sequence of SEQ ID NO:133 or a sequence having a sequence identity of at least 80%, more preferably at least 85% more preferably at least 90%, more preferably at least 93% with SEQ ID NO:133.

All primers indicated under points i. to o. and (VI) to (XIII) above may comprise a sequence having at least 80%, more preferably at least 85%, more preferably at least 90%, more preferably at least 93% sequence identity with the sequences of the indicated SEQ ID NOs.

In embodiments of the present invention, the extension primers do not only comprise the sequences indicated above or variants having a sequence identity of at least 80%, more preferably at least 85% more preferably at least 90%, more preferably at least 93% therewith but even consist of these sequences. However, the present inventors found that it may be advantageous if non-complementary bases are added at the 5′-end of the primers so that each primer has a detectable different mass.

A particularly preferred kit of the present invention comprises
(ii) at least one, more preferably at least two, more preferably at least three, more preferably at least four, more preferably at least five, more preferably at least six and even more preferably all seven of the following extension primers:
   i. gapA (420) extension primer comprising a sequence of SEQ ID NO:27 or a sequence having a sequence identity of at least 80%, more preferably at least 85% more preferably at least 90%, more preferably at least 93% with SEQ ID NO:27,
   j. gapA (456) extension primer comprising a sequence of SEQ ID NO:30 or a sequence having a sequence identity of at least 80%, more preferably at least 85% more preferably at least 90%, more preferably at least 93% with SEQ ID NO:30,
   k. infB (1608) extension primer comprising a sequence of SEQ ID NO:33 or a sequence having a sequence identity of at least 80%, more preferably at least 85% more preferably at least 90%, more preferably at least 93% with SEQ ID NO:33,
   l. mdh (633) extension primer comprising a sequence of SEQ ID NO:36 or a sequence having a sequence identity of at least 80%, more preferably at least 85% more preferably at least 90%, more preferably at least 93% with SEQ ID NO:36,
   m. phoE (732) extension primer comprising a sequence of SEQ ID NO:39 or a sequence having a sequence identity of at least 80%, more preferably at least 85% more preferably at least 90%, more preferably at least 93% with SEQ ID NO:39,
   n. phoE (867) extension primer comprising a sequence of SEQ ID NO:42 or a sequence having a sequence identity of at least 80%, more preferably at least 85% more preferably at least 90%, more preferably at least 93% with SEQ ID NO:42,
   o. rpoB (1819) extension primer comprising a sequence of SEQ ID NO:45 or a sequence having a sequence identity of at least 80%, more preferably at least 85% more preferably at least 90%, more preferably at least 93% with SEQ ID NO:45.

Additionally or alternatively to extension primers i. to o. indicated above, the kit preferably further comprises extension primers corresponding to primer pairs 20 to 27. Preferably, the kit comprises
(ii) at least one, more preferably at least two, more preferably at least three, more preferably at least four, more preferably at least five, more preferably at least six, more preferably at least seven, more preferably all eight of the following extension primers:
   (VI) gyrB extension primer comprising a sequence of SEQ ID NO:112 or a sequence having a sequence identity of at least 80%, more preferably at least 85% more preferably at least 90%, more preferably at least 93% with SEQ ID NO:112,
   (VII) adk (348) extension primer comprising a sequence of SEQ ID NO:115 or a sequence having a sequence identity of at least 80%, more preferably at least 85% more preferably at least 90%, more preferably at least 93% with SEQ ID NO:115,
   (VIII) adk (456) extension primer comprising a sequence of SEQ ID NO:118 or a sequence having a sequence identity of at least 80%, more preferably at least 85% more preferably at least 90%, more preferably at least 93% with SEQ ID NO:118,
   (IX) icd (243) extension primer comprising a sequence of SEQ ID NO:121 or a sequence having a sequence identity of at least 80%, more preferably at least 85% more preferably at least 90%, more preferably at least 93% with SEQ ID NO:121,
   (X) icd (606) extension primer comprising a sequence of SEQ ID NO:124 or a sequence having a sequence identity of at least 80%, more preferably at least 85% more preferably at least 90%, more preferably at least 93% with SEQ ID NO:124,
   (XI) purA extension primer comprising a sequence of SEQ ID NO:127 or a sequence having a sequence identity of at least 80%, more preferably at least 85% more preferably at least 90%, more preferably at least 93% with SEQ ID NO:127,
   (XII) lexA extension primer comprising a sequence of SEQ ID NO:130 or a sequence having a sequence identity of at least 80%, more preferably at least 85% more preferably at least 90%, more preferably at least 93% with SEQ ID NO:130,
   (XIII) rpoH extension primer comprising a sequence of SEQ ID NO:133 or a sequence having a sequence identity of at least 80%, more preferably at least 85% more preferably at least 90%, more preferably at least 93% with SEQ ID NO:133.

All primers indicated under points i. to o. and (VI) to (XIII) above may comprise a sequence having at least 80%, more preferably at least 85%, more preferably at least 90%, more preferably at least 94% sequence identity with the sequences of the indicated SEQ ID NOs.

Preferably, the extension primers indicated above do not only comprise the sequences indicated above or variants having a sequence identity of at least 80%, more preferably at least 85% more preferably at least 90%, more preferably at least 93% therewith but even consist of these sequences.

A particularly preferred kit of the present invention comprises
(i) the following amplification primers:
   a. A forward primer comprising a sequence of SEQ ID NO:1 or a sequence having at least 80%, preferably at least 85%, more preferably at least 90%, more preferably at least 95% sequence identity therewith, and a reverse primer comprising a sequence of SEQ ID NO:2 or a sequence having at least 80%, preferably at least 85%, more preferably at least 90%, more preferably at least 95% sequence identity therewith,
   b. A forward primer comprising a sequence of SEQ ID NO:4 or a sequence having at least 80%, preferably at least 85%, more preferably at least 90%, more preferably at least 95% sequence identity therewith, and a reverse primer comprising a sequence of SEQ ID NO:5 or a sequence having at least 80%, preferably at least 85%, more preferably at least 90%, more preferably at least 95% sequence identity therewith,
   c. A forward primer comprising a sequence of SEQ ID NO:7 or a sequence having at least 80%, preferably at least 85%, more preferably at least 90%, more preferably at least 95% sequence identity therewith, and a reverse primer comprising a sequence of SEQ ID NO:8 or a sequence having at least 80%, preferably at least 85%, more preferably at least 90%, more preferably at least 95% sequence identity therewith,
   d. A forward primer comprising a sequence of SEQ ID NO:10 or a sequence having at least 80%, preferably at least 85%, more preferably at least 90%, more preferably at least 95% sequence identity therewith, and a reverse primer comprising a sequence of SEQ ID NO:11 or a sequence having at least 80%, preferably at least 85%, more preferably at least 90%, more preferably at least 95% sequence identity therewith,
   e. A forward primer comprising a sequence of SEQ ID NO:13 or a sequence having at least 80%, preferably at least 85%, more preferably at least 90%, more preferably at least 95% sequence identity therewith, and a reverse primer comprising a sequence of SEQ ID NO:14 or a sequence having at least 80%, preferably at least 85%, more preferably at least 90%, more preferably at least 95% sequence identity therewith,
   f. A forward primer comprising a sequence of SEQ ID NO:16 or a sequence having at least 80%, preferably at least 85%, more preferably at least 90%, more preferably at least 95% sequence identity therewith, and a reverse primer comprising a sequence of SEQ ID NO:17 or a sequence having at least 80%, preferably at least 85%, more preferably at least 90%, more preferably at least 95% sequence identity therewith,
   g. A forward primer comprising a sequence of SEQ ID NO:19 or a sequence having at least 80%, preferably at least 85%, more preferably at least 90%, more preferably at least 95% sequence identity therewith, and a reverse primer comprising a sequence of SEQ ID NO:20 or a sequence having at least 80%, preferably at least 85%, more preferably at least 90%, more preferably at least 95% sequence identity therewith,
   h. A forward primer comprising a sequence of SEQ ID NO:22 or a sequence having at least 80%, preferably at least 85%, more preferably at least 90%, more preferably at least 95% sequence identity therewith, and a reverse primer comprising a sequence of SEQ ID NO:23 or a sequence having at least 80%, preferably at least 85%, more preferably at least 90%, more preferably at least 95% sequence identity therewith,
   i. A forward primer comprising a sequence of SEQ ID NO:25 or a sequence having at least 80%, preferably at least 85%, more preferably at least 90%, more preferably at least 95% sequence identity therewith, and a reverse primer comprising a sequence of SEQ ID NO:26 or a sequence having at least 80%, preferably at least 85%, more preferably at least 90%, more preferably at least 95% sequence identity therewith,
   j. A forward primer comprising a sequence of SEQ ID NO:31 or a sequence having at least 80%, preferably at least 85%, more preferably at least 90%, more preferably at least 95% sequence identity therewith, and a reverse primer comprising a sequence of SEQ ID NO:32 or a sequence having at least 80%, preferably at least 85%, more preferably at least 90%, more preferably at least 95% sequence identity therewith,
   k. A forward primer comprising a sequence of SEQ ID NO:34 or a sequence having at least 80%, preferably at least 85%, more preferably at least 90%, more preferably at least 95% sequence identity therewith, and a reverse primer comprising a sequence of SEQ ID NO:35 or a sequence having at least 80%, preferably at least 85%, more preferably at least 90%, more preferably at least 95% sequence identity therewith,
   l. A forward primer comprising a sequence of SEQ ID NO:37 or a sequence having at least 80%, preferably at least 85%, more preferably at least 90%, more preferably at least 95% sequence identity therewith, and a reverse primer comprising a sequence of SEQ ID NO:38 or a sequence having at least 80%, preferably at least 85%, more preferably at least 90%, more preferably at least 95% sequence identity therewith,
   m. A forward primer comprising a sequence of SEQ ID NO:40 or a sequence having at least 80%, preferably at least 85%, more preferably at least 90%, more preferably at least 95% sequence identity therewith, and a reverse primer comprising a sequence of SEQ ID NO:41 or a sequence having at least 80%, preferably at least 85%, more preferably at least 90%, more preferably at least 95% sequence identity therewith, and
   n. A forward primer comprising a sequence of SEQ ID NO:43 or a sequence having at least 80%, preferably at least 85%, more preferably at least 90%, more preferably at least 95% sequence identity therewith, and a reverse primer comprising a sequence of SEQ ID NO:44 or a sequence having at least 80%, preferably at least 85%, more preferably at least 90%, more preferably at least 95% sequence identity therewith, and
(ii) the following extension primers:
   a. KPC-2 extension primer comprising a sequence of SEQ ID NO:3 or a sequence having a sequence identity of at least 80%, more preferably at least 85% more preferably at least 90%, more preferably at least 93% with SEQ ID NO:3,
   b. NDM-1 extension primer comprising a sequence of SEQ ID NO:6 or a sequence having a sequence identity of at least 80%, more preferably at least 85% more preferably at least 90%, more preferably at least 93% with SEQ ID NO:6,
   c. OXA-9 extension primer comprising a sequence of SEQ ID NO:9 or a sequence having a sequence identity of at least 80%, more preferably at least 85% more preferably at least 90%, more preferably at least 93% with SEQ ID NO:9,
   d. OXA-48 extension primer comprising a sequence of SEQ ID NO:12 or a sequence having a sequence identity of at least 80%, more preferably at least 85% more preferably at least 90%, more preferably at least 93% with SEQ ID NO:12,
   e. CTX-M-9 extension primer comprising a sequence of SEQ ID NO:15 or a sequence having a sequence identity of at least 80%, more preferably at least 85% more preferably at least 90%, more preferably at least 93% with SEQ ID NO:15,
   f. CTX-M-15 extension primer comprising a sequence of SEQ ID NO:18 or a sequence having a sequence identity of at least 80%, more preferably at least 85% more preferably at least 90%, more preferably at least 93% with SEQ ID NO:18,
   g. AAC(6′)-lb-cr (223) extension primer comprising a sequence of SEQ ID NO:21 or a sequence having a sequence identity of at least 80%, more preferably at least 85% more preferably at least 90%, more preferably at least 93% with SEQ ID NO:21,
   h. AAC(6′)-lb-cr (454) extension primer comprising a sequence of SEQ ID NO:24 or a sequence having a sequence identity of at least 80%, more preferably at least 85% more preferably at least 90%, more preferably at least 93% with SEQ ID NO:24,
   i. gapA (420) extension primer comprising a sequence of SEQ ID NO:27 or a sequence having a sequence identity of at least 80%, more preferably at least 85% more preferably at least 90%, more preferably at least 93% with SEQ ID NO:27,
   j. gapA (456) extension primer comprising a sequence of SEQ ID NO:30 or a sequence having a sequence identity of at least 80%, more preferably at least 85% more preferably at least 90%, more preferably at least 93% with SEQ ID NO:30,
   k. infB (1608) extension primer comprising a sequence of SEQ ID NO:33 or a sequence having a sequence identity of at least 80%, more preferably at least 85% more preferably at least 90%, more preferably at least 93% with SEQ ID NO:33,
   l. mdh (633) extension primer comprising a sequence of SEQ ID NO:36 or a sequence having a sequence identity of at least 80%, more preferably at least 85% more preferably at least 90%, more preferably at least 93% with SEQ ID NO:36,
   m. phoE (732) extension primer comprising a sequence of SEQ ID NO:39 or a sequence having a sequence identity of at least 80%, more preferably at least 85% more preferably at least 90%, more preferably at least 93% with SEQ ID NO:39,
   n. phoE (867) extension primer comprising a sequence of SEQ ID NO:42 or a sequence having a sequence identity of at least 80%, more preferably at least 85% more preferably at least 90%, more preferably at least 93% with SEQ ID NO:42, and
   o. rpoB (1819) extension primer comprising a sequence of SEQ ID NO:45 or a sequence having a sequence identity of at least 80%, more preferably at least 85% more preferably at least 90%, more preferably at least 93% with SEQ ID NO:45.

Preferably, the kit further comprises
(i) the following amplification primers:
   (I) A forward primer comprising a sequence of SEQ ID NO:95 or a sequence having at least 80%, preferably at least 85%, more preferably at least 90%, more preferably at least 95% sequence identity therewith, and a reverse primer comprising a sequence of SEQ ID NO:96 or a sequence having at least 80%, preferably at least 85%, more preferably at least 90%, more preferably at least 95% sequence identity therewith,
   (II) A forward primer comprising a sequence of SEQ ID NO:98 or a sequence having at least 80%, preferably at least 85%, more preferably at least 90%, more preferably at least 95% sequence identity therewith, and a reverse primer comprising a sequence of SEQ ID NO:99 or a sequence having at least 80%, preferably at least 85%, more preferably at least 90%, more preferably at least 95% sequence identity therewith,
   (III) A forward primer comprising a sequence of SEQ ID NO:101 or a sequence having at least 80%, preferably at least 85%, more preferably at least 90%, more preferably at least 95% sequence identity therewith, and a reverse primer comprising a sequence of SEQ ID NO:102 or a sequence having at least 80%, preferably at least 85%, more preferably at least 90%, more preferably at least 95% sequence identity therewith,
   (IV) A forward primer comprising a sequence of SEQ ID NO:104 or a sequence having at least 80%, preferably at least 85%, more preferably at least 90%, more preferably at least 95% sequence identity therewith, and a reverse primer comprising a sequence of SEQ ID NO:105 or a sequence having at least 80%, preferably at least 85%, more preferably at least 90%, more preferably at least 95% sequence identity therewith,
   (V) A forward primer comprising a sequence of SEQ ID NO:107 or a sequence having at least 80%, preferably at least 85%, more preferably at least 90%, more preferably at least 95% sequence identity therewith, and a reverse primer comprising a sequence of SEQ ID NO:108 or a sequence having at least 80%, preferably at least 85%, more preferably at least 90%, more preferably at least 95% sequence identity therewith,
   (VI) A forward primer comprising a sequence of SEQ ID NO:110 or a sequence having at least 80%, preferably at least 85%, more preferably at least 90%, more preferably at least 95% sequence identity therewith, and a reverse primer comprising a sequence of SEQ ID NO:111 or a sequence having at least 80%, preferably at least 85%, more preferably at least 90%, more preferably at least 95% sequence identity therewith,
   (VII) A forward primer comprising a sequence of SEQ ID NO:113 or a sequence having at least 80%, preferably at least 85%, more preferably at least 90%, more preferably at least 95% sequence identity therewith, and a reverse primer comprising a sequence of SEQ ID NO:114 or a sequence having at least 80%, preferably at least 85%, more preferably at least 90%, more preferably at least 95% sequence identity therewith,
   (VIII) A forward primer comprising a sequence of SEQ ID NO:116 or a sequence having at least 80%, preferably at least 85%, more preferably at least 90%, more preferably at least 95% sequence identity therewith, and a reverse primer comprising a sequence of SEQ ID NO:117 or a sequence having at least 80%, preferably at least 85%, more preferably at least 90%, more preferably at least 95% sequence identity therewith,
   (IX) A forward primer comprising a sequence of SEQ ID NO:119 or a sequence having at least 80%, preferably at least 85%, more preferably at least 90%, more preferably at least 95% sequence identity therewith, and a reverse primer comprising a sequence of SEQ ID NO:120 or a sequence having at least 80%, preferably at least 85%, more preferably at least 90%, more preferably at least 95% sequence identity therewith,
   (X) A forward primer comprising a sequence of SEQ ID NO:122 or a sequence having at least 80%, preferably at least 85%, more preferably at least 90%, more preferably at least 95% sequence identity therewith, and a reverse primer comprising a sequence of SEQ ID NO:123 or a sequence having at least 80%, preferably at least 85%, more preferably at least 90%, more preferably at least 95% sequence identity therewith,
   (XI) A forward primer comprising a sequence of SEQ ID NO:125 or a sequence having at least 80%, preferably at least 85%, more preferably at least 90%, more preferably at least 95% sequence identity therewith, and a reverse primer comprising a sequence of SEQ ID NO:126 or a sequence having at least 80%, preferably at least 85%, more preferably at least 90%, more preferably at least 95% sequence identity therewith,
   (XII) A forward primer comprising a sequence of SEQ ID NO:128 or a sequence having at least 80%, preferably at least 85%, more preferably at least 90%, more preferably at least 95% sequence identity therewith, and a reverse primer comprising a sequence of SEQ ID NO:129 or a sequence having at least 80%, preferably at least 85%, more preferably at least 90%, more preferably at least 95% sequence identity therewith,
   (XIII) A forward primer comprising a sequence of SEQ ID NO:131 or a sequence having at least 80%, preferably at least 85%, more preferably at least 90%, more preferably at least 95% sequence identity therewith, and a reverse primer comprising a sequence of SEQ ID NO:132 or a sequence having at least 80%, preferably at least 85%, more preferably at least 90%, more preferably at least 95% sequence identity therewith, and
(ii) the following extension primers
   (I) OXA-1 extension primer comprising a sequence of SEQ ID NO:97 or a sequence having a sequence identity of at least 80%, more preferably at least 85% more preferably at least 90%, more preferably at least 93% with SEQ ID NO:97,
   (II) OXA-2 extension primer comprising a sequence of SEQ ID NO:100 or a sequence having a sequence identity of at least 80%, more preferably at least 85% more preferably at least 90%, more preferably at least 93% with SEQ ID NO:100,
   (III) TEM-1 extension primer comprising a sequence of SEQ ID NO:103 or a sequence having a sequence identity of at least 80%, more preferably at least 85% more preferably at least 90%, more preferably at least 93% with SEQ ID NO:103,
   (IV) APHA3-la extension primer comprising a sequence of SEQ ID NO:106 or a sequence having a sequence identity of at least 80%, more preferably at least 85% more preferably at least 90%, more preferably at least 93% with SEQ ID NO:106,
   (V) AAC(3′)-IIae extension primer comprising a sequence of SEQ ID NO:109 or a sequence having a sequence identity of at least 80%, more preferably at least 85% more preferably at least 90%, more preferably at least 93% with SEQ ID NO:109,
   (VI) gyrB extension primer comprising a sequence of SEQ ID NO:112 or a sequence having a sequence identity of at least 80%, more preferably at least 85% more preferably at least 90%, more preferably at least 93% with SEQ ID NO:112,
   (VII) adk (348) extension primer comprising a sequence of SEQ ID NO:115 or a sequence having a sequence identity of at least 80%, more preferably at least 85% more preferably at least 90%, more preferably at least 93% with SEQ ID NO:115,
   (VIII) adk (456) extension primer comprising a sequence of SEQ ID NO:118 or a sequence having a sequence identity of at least 80%, more preferably at least 85% more preferably at least 90%, more preferably at least 93% with SEQ ID NO:118,
   (IX) icd (243) extension primer comprising a sequence of SEQ ID NO:121 or a sequence having a sequence identity of at least 80%, more preferably at least 85% more preferably at least 90%, more preferably at least 93% with SEQ ID NO:121,
   (X) icd (606) extension primer comprising a sequence of SEQ ID NO:124 or a sequence having a sequence identity of at least 80%, more preferably at least 85% more preferably at least 90%, more preferably at least 93% with SEQ ID NO:124,
   (XI) purA extension primer comprising a sequence of SEQ ID NO:127 or a sequence having a sequence identity of at least 80%, more preferably at least 85% more preferably at least 90%, more preferably at least 93% with SEQ ID NO:127,
   (XII) lexA extension primer comprising a sequence of SEQ ID NO:130 or a sequence having a sequence identity of at least 80%, more preferably at least 85% more preferably at least 90%, more preferably at least 93% with SEQ ID NO:130, and
   (XIII) rpoH extension primer comprising a sequence of SEQ ID NO:133 or a sequence having a sequence identity of at least 80%, more preferably at least 85% more preferably at least 90%, more preferably at least 93% with SEQ ID NO:133.

All primers of the primer pairs indicated under points a. to n. (I) to (XIII) above may comprise a sequence having at least 80%, more preferably at least 85%, more preferably at least 90%, more preferably at least 95% sequence identity with the sequences of the indicated SEQ ID NOs and all extension primers indicated under points a. to o. (I) to (XIII) above may comprise a sequence having at least 80%, more preferably at least 85%, more preferably at least 90%, more preferably at least 93% sequence identity with the sequences of the indicated SEQ ID NOs.

Preferably, the primers indicated above do not only comprise the sequences indicated above or variants having the indicated sequence identity therewith but even consist of these sequences.

The kits of the present invention may further comprise reagents for performing a PCR reaction. Preferably, the kit further comprises at least one reagent selected from the group consisting of buffer solution, deoxynucleotides (dNTPs) and DNA polymerase. More preferably, the kit comprises buffer solution, deoxynucleotides (dNTPs) and DNA polymerase. Preferably, the DNA polymerase is selected from the group consisting of Taq polymerase, Pwo polymerase and Pfu polymerase. Pfu polymerase is most preferred because it creates very little mutations during DNA amplification.

In a particularly preferred embodiment, the kit further comprises chain terminating nucleotides. Preferred chain terminating nucleotides of the present invention are acyclonucleotides (acyNTPs). Acyclonucleotides enable elongation of extension primers by exactly one nucleotide. In an alternative embodiment, the kit may comprise ddNTPs as chain terminating nucleotides instead of acyNTPs.

Preferably, the kit further comprises an archaeon DNA polymerase, more preferably Therminator DNA Polymerase.

The present invention also comprises the use of the above described methods for determining from a bacterial sample information on both the phylogenetic origin and the antibiotic resistances of bacterial strains.

The present invention also comprises a method of diagnosis of a disease based on the information on both the phylogenetic origin and/or antibiotic resistances of the bacterial strain determined by the methods of the present invention described above. The method of diagnosis of a disease preferably comprises the step of identifying the disease based on the determined information on both the phylogenetic origin and antibiotic resistances of the bacterial strain.

The present invention also comprises a method of treatment of a disease comprising the step of identifying a suitable drug for treatment of the disease based on the diagnosis of the disease by means of the method of the present invention and preferably the additional step of administering the identified drug to a patient in need thereof.

The present invention also comprises a method of infection management comprising the step of tracking the course of infection spreading based on the information on the phylogenetic origin and/or antibiotic resistances of the bacterial strain determined by the methods of the present invention described above. Preferably, the method comprises the additional step of improving hygienic standards based on the information obtained on the course of infection spreading.

### EXAMPLES

### Example 1

The 8 universal resistance marker SNPs of the present invention (position 814 of the KPC-2 coding sequence (SEQ ID NO:87), position 82 of the NDM-1 coding sequence (SEQ ID NO:88), position 104 of the OXA-9 coding sequence (SEQ ID NO:89), position 186 of the OXA-48 coding sequence (SEQ ID NO:90), position 556 of the CTX-M-9 coding sequence (SEQ ID NO:91), position 453 of the CTX-M-15 coding sequence (SEQ ID NO:92), position 223 of the AAC(6′)-lb-cr coding sequence (SEQ ID NO:93) and position 454 of the AAC(6′)-lb-cr coding sequence (SEQ ID NO:93)) have been tested for their ability to enable phylogenetic analysis. 93 sequenced K. pneumoniae isolates were investigated.

PCR reactions comprising the following primer pairs were performed:
a. Primer pair 1 consisting of a forward primer consisting of a sequence of SEQ ID NO:1 and a reverse primer consisting of a sequence of SEQ ID NO:2,
b. Primer pair 2 consisting of a forward primer consisting of a sequence of SEQ ID NO:4 and a reverse primer consisting of a sequence of SEQ ID NO:5,
c. Primer pair 3 consisting of a forward primer consisting of a sequence of SEQ ID NO:7 and a reverse primer consisting of a sequence of SEQ ID NO:8,
d. Primer pair 4 consisting of a forward primer consisting of a sequence of SEQ ID NO:10 and a reverse primer consisting of a sequence of SEQ ID NO:11,
e. Primer pair 5 consisting of a forward primer consisting of a sequence of SEQ ID NO:13 and a reverse primer consisting of a sequence of SEQ ID NO:14,
f. Primer pair 6 consisting of a forward primer consisting of a sequence of SEQ ID NO:16 and a reverse primer consisting of a sequence of SEQ ID NO:17,
g. Primer pair 7 consisting of a forward primer consisting of a sequence of SEQ ID NO:19 and a reverse primer consisting of a sequence of SEQ ID NO:20, and
h. Primer pair 8 consisting of a forward primer consisting of a sequence of SEQ ID NO:22 and a reverse primer consisting of a sequence of SEQ ID NO:23.

Extension primers were allowed to bind to the PCR products obtained from the PCR reactions. In particular,
a. KPC-2 extension primer consisting of a sequence of SEQ ID NO:3 was allowed to bind to the PCR product of primer pair 1,
b. NDM-1 extension primer consisting of a sequence of SEQ ID NO:6 was allowed to bind to the PCR product of primer pair 2,
c. OXA-9 extension primer consisting of a sequence of SEQ ID NO:9 was allowed to bind to the PCR product of primer pair 3,
d. OXA-48 extension primer consisting of a sequence of SEQ ID NO:12 was allowed to bind to the PCR product of primer pair 4,
e. CTX-M-9 extension primer consisting of a sequence of SEQ ID NO:15 was allowed to bind to the PCR product of primer pair 5,
f. CTX-M-15 extension primer consisting of a sequence of SEQ ID NO:18 was allowed to bind to the PCR product of primer pair 6,
g. AAC(6′)-lb-cr (223) extension primer consisting of a sequence of SEQ ID NO:21 was allowed to bind to the PCR product of primer pair 7, and
h. AAC(6′)-lb-cr (454) extension primer consisting of a sequence of SEQ ID NO:24 was allowed to bind to the PCR product of primer pair 8.

Extension primers were elongated at the 3′-end by exactly one nucleotide. The elongated extension primers were analyzed by mass spectrometry and the nucleotide that has been added to the extension primers was determined.

Information on both the phylogenetic origin and antibiotic resistances of the bacterial strains was determined based on a comparison of the pattern of the determined nucleotides with respective predetermined patterns of bacterial strains, which phylogenetic origin and antibiotic resistances were known.

A phylogenetic tree was generated in MEGA6 using Neighbor Joining without bootstrapping. The resulting tree is shown in FIG. 3. The tree is based only on the resistance markers (SNP) including information about presence and absence of genes. The data show that information on the phylogenetic origin is derived from the analysis of the resistance markers of the present invention. Not every isolate was distinguishable but a satisfactory phylogenetic determination was achieved.

The analysis of the resistance markers of the present invention can be used to identify common and divergent patterns between different strains that were subjected to the analysis of the resistance markers. This information can be used to identify the phylogenetic relatedness of the strains. Many strains can be distinguished by the use of the resistance markers (SNP) including information about presence and absence of genes, however for some of the strains this information is not sufficient to differentiate them.

### Example 2

In another experiment, 7 phylogenetic Klebsiella markers (nucleotide at position 420 of the gapA coding sequence (SEQ ID NO:28), position 456 of the gapA coding sequence (SEQ ID NO:28), position 1608 of the infB coding sequence (SEQ ID NO:29), position 633 of the mdh coding sequence (SEQ ID NO:70), position 732 of the phoE coding sequence (SEQ ID NO:71), position 867 of the phoE coding sequence (SEQ ID NO:71) and position 1819 of the rpoB coding sequence (SEQ ID NO:94)) were tested for their discriminative power with regard to the 93 sequenced K. pneumoniae isolates described in example 1.

PCR reactions comprising the following primer pairs were performed:
i. Primer pair 9 consisting of a forward primer consisting of a sequence of SEQ ID NO:25 and a reverse primer consisting of a sequence of SEQ ID NO:26,
j. Primer pair 10 consisting of a forward primer consisting of a sequence of SEQ ID NO:31 and a reverse primer consisting of a sequence of SEQ ID NO:32,
k. Primer pair 11 consisting of a forward primer consisting of a sequence of SEQ ID NO:34 and a reverse primer consisting of a sequence of SEQ ID NO:35,
l. Primer pair 12 consisting of a forward primer consisting of a sequence of SEQ ID NO:37 and a reverse primer consisting of a sequence of SEQ ID NO:38,
m. Primer pair 13 consisting of a forward primer consisting of a sequence of SEQ ID NO:40 and a reverse primer consisting of a sequence of SEQ ID NO:41, and
n. Primer pair 14 consisting of a forward primer consisting of a sequence of SEQ ID NO:43 and a reverse primer consisting of a sequence of SEQ ID NO:44.

Extension primers were allowed to bind to the PCR products obtained from the PCR reactions. In particular,
i. gapA (420) extension primer consisting of a sequence of SEQ ID NO:27 was allowed to bind to the PCR product of primer pair 9,
j. gapA (456) extension primer consisting of a sequence of SEQ ID NO:30 was allowed to bind to the PCR product of primer pair 9,
k. infB (1608) extension primer consisting of a sequence of SEQ ID NO:33 was allowed to bind to the PCR product of primer pair 10,
l. mdh (633) extension primer consisting of a sequence of SEQ ID NO:36 was allowed to bind to the PCR product of primer pair 11,
m. phoE (732) extension primer consisting of a sequence of SEQ ID NO:39 was allowed to bind to the PCR product of primer pair 12,
n. phoE (867) extension primer consisting of a sequence of SEQ ID NO:42 was allowed to bind to the PCR product of primer pair 13, and
o. rpoB (1819) extension primer consisting of a sequence of SEQ ID NO:45 was allowed to bind to the PCR product of primer pair 14.

Extension primers were elongated at the 3′-end by exactly one nucleotide. The elongated extension primers were analyzed by mass spectrometry and the nucleotide that has been added to the extension primers was determined.

Information on the phylogenetic origin of the bacterial strains was determined based on a comparison of the pattern of the determined nucleotides with respective predetermined patterns of bacterial strains, which phylogenetic origin was known.

A phylogenetic tree was generated in MEGA6 using Neighbor Joining without bootstrapping. The resulting tree is shown in FIG. 4. The tree is based only on the phylogeny markers (SNP) including information about presence and absence of genes. As compared to example 1, testing the 7 phylogenetic markers results in an improved resolution of the phylogenetic analysis. However, also with the phylogenetic markers the results were not optimal. In particular, several distinct isolates were not distinguishable

### Example 3

A combined analysis utilizing information from both the 8 universal resistance markers and the 7 phylogenetic markers was performed. 93 sequenced K. pneumoniae isolates were analyzed as described in examples 1 and 2. A phylogenetic tree was generated in MEGA6 using Neighbor Joining without bootstrapping. The resulting tree is shown in FIG. 5. The tree is based on both the resistance markers and the phylogeny markers including information about presence and absence of genes. The data show that combined testing of the 8 resistance markers and the 7 phylogenetic markers results in further improvement of the resolution of the phylogenetic analysis as compared to the results of example 2.

### Example 4

The 8 universal resistance markers (position 814 of the KPC-2 coding sequence (SEQ ID NO:87), position 82 of the NDM-1 coding sequence (SEQ ID NO:88), position 104 of the OXA-9 coding sequence (SEQ ID NO:89), position 186 of the OXA-48 coding sequence (SEQ ID NO:90), position 556 of the CTX-M-9 coding sequence (SEQ ID NO:91), position 453 of the CTX-M-15 coding sequence (SEQ ID NO:92), position 223 of the AAC(6′)-lb-cr coding sequence (SEQ ID NO:93) and position 454 of the AAC(6′)-lb-cr coding sequence (SEQ ID NO:93)) were used for investigation of 55 sequenced E. coli isolates. PCR reactions were performed as described in example 1 using the same primers as described in example 1. The nucleotides of interest were identified by mass spectrometry as described in example 1. 5 additional universal resistance markers were used in order to further improve the phylogenetic differentiation for E. coli. Namely, position 408 of the OXA-1 coding sequence (SEQ ID NO:163), position 585 of the OXA-2 coding sequence (SEQ ID NO:164), position 228 of the TEM-1 coding sequence (SEQ ID NO:165), position 230 of the APHA3-la coding sequence (SEQ ID NO:166) and position 63 of the AAC(3′)-IIae coding sequence (SEQ ID NO:167) have been tested for their ability to further improve phylogenetic analysis of the 55 sequenced E. coli isolates.

PCR reactions comprising the following primer pairs were performed:
(I) Primer pair 1 consisting of a forward primer consisting of a sequence of SEQ ID NO:95 and a reverse primer consisting of a sequence of SEQ ID NO:96,
(II) Primer pair 2 consisting of a forward primer consisting of a sequence of SEQ ID NO:98 and a reverse primer consisting of a sequence of SEQ ID NO:99,
(III) Primer pair 3 consisting of a forward primer consisting of a sequence of SEQ ID NO:101 and a reverse primer consisting of a sequence of SEQ ID NO:102,
(IV) Primer pair 4 consisting of a forward primer consisting of a sequence of SEQ ID NO:104 and a reverse primer consisting of a sequence of SEQ ID NO:105,
(V) Primer pair 5 consisting of a forward primer consisting of a sequence of SEQ ID NO:107 and a reverse primer consisting of a sequence of SEQ ID NO:108.

Extension primers were allowed to bind to the PCR products obtained from the PCR reactions. In particular,
(I) OXA-1 extension primer consisting of a sequence of SEQ ID NO:97 was allowed to bind to the PCR product of primer pair 1,
(II) OXA-2 extension primer consisting of a sequence of SEQ ID NO:100 was allowed to bind to the PCR product of primer pair 2,
(III) TEM-1 extension primer consisting of a sequence of SEQ ID NO:103 was allowed to bind to the PCR product of primer pair 3,
(IV) APHA3-la extension primer consisting of a sequence of SEQ ID NO:106 was allowed to bind to the PCR product of primer pair 4,
(V) AAC(3′)-IIae extension primer consisting of a sequence of SEQ ID NO:109 was allowed to bind to the PCR product of primer pair 5.

Extension primers were elongated at the 3′-end by exactly one nucleotide. The elongated extension primers were analyzed by mass spectrometry and the nucleotide that has been added to the extension primers was determined.

Information on both the phylogenetic origin and antibiotic resistances of the bacterial strains was determined based on a comparison of the pattern of the determined nucleotides with respective predetermined patterns of bacterial strains, which phylogenetic origin and antibiotic resistances were known.

An alignment of the concatenated sequence of determined nucleotides for the 8 resistance markers and an additional analysis containing all 13 resistance markers was generated with ClustalW version 2.1 and a phylogenetic tree was generated using default settings and visualized with iTol (http://itol.embl.de/). The resulting tree for the previous 8 resistance markers is shown in FIG. 8 and the phylogenetic tree for all 13 resistance markers is shown in FIG. 9. The trees are based only on the resistance markers (SNP) including information about presence and absence of genes. The data show that information on the phylogenetic origin is derived from the analysis of the resistance markers of the present invention. Not every isolate was distinguishable, but a satisfactory phylogenetic determination was achieved.

The analysis of the resistance markers of the present invention can be used to identify common and divergent patterns between different strains that were subjected to the analysis of the resistance markers. This information can be used to identify the phylogenetic relatedness of the strains. Many strains can be distinguished by the use of the resistance markers (SNP) including information about presence and absence of genes, however, for some of the strains this information is not sufficient to differentiate them.

### Example 5

In another experiment, 8 phylogenetic E. coli markers (nucleotide at position 456 of the gyrB coding sequence (SEQ ID NO:168), position 348 of the adk coding sequence (SEQ ID NO:169), position 456 of the adk coding sequence (SEQ ID NO:169), position 243 of the icd coding sequence (SEQ ID NO:170), position 606 of the icd coding sequence (SEQ ID NO:170), position 426 of the purA coding sequence (SEQ ID NO:171), position 384 of the lexA coding sequence (SEQ ID NO:172) and position 279 of the rpoH coding sequence (SEQ ID NO:173)) were tested for their discriminative power with regard to the 55 sequenced E. coli isolates described in example 4.

PCR reactions comprising the following primer pairs were performed:
(VI) Primer pair 6 consisting of a forward primer consisting of a sequence of SEQ ID NO:110 and a reverse primer consisting of a sequence of SEQ ID NO:111,
(VII) Primer pair 7 consisting of a forward primer consisting of a sequence of SEQ ID NO:113 and a reverse primer consisting of a sequence of SEQ ID NO:114,
(VIII) Primer pair 8 consisting of a forward primer consisting of a sequence of SEQ ID NO:116 and a reverse primer consisting of a sequence of SEQ ID NO:117,
(IX) Primer pair 9 consisting of a forward primer consisting of a sequence of SEQ ID NO:119 and a reverse primer consisting of a sequence of SEQ ID NO:120,
(X) Primer pair 10 consisting of a forward primer consisting of a sequence of SEQ ID NO:122 and a reverse primer consisting of a sequence of SEQ ID NO:123, and
(XI) Primer pair 11 consisting of a forward primer consisting of a sequence of SEQ ID NO:125 and a reverse primer consisting of a sequence of SEQ ID NO:126.
(XII) Primer pair 12 consisting of a forward primer consisting of a sequence of SEQ ID NO:128 and a reverse primer consisting of a sequence of SEQ ID NO:129
(XIII) Primer pair 13 consisting of a forward primer consisting of a sequence of SEQ ID NO:131 and a reverse primer consisting of a sequence of SEQ ID NO:132

Extension primers were allowed to bind to the PCR products obtained from the PCR reactions. In particular,
(VI) gyrB (456) extension primer consisting of a sequence of SEQ ID NO:112 was allowed to bind to the PCR product of primer pair 6,
(VII) adk (348) extension primer consisting of a sequence of SEQ ID NO:115 was allowed to bind to the PCR product of primer pair 7,
(VIII) adk (456) extension primer consisting of a sequence of SEQ ID NO:118 was allowed to bind to the PCR product of primer pair 8,
(IX) icd (243) extension primer consisting of a sequence of SEQ ID NO:121 was allowed to bind to the PCR product of primer pair 9,
(X) icd (606) extension primer consisting of a sequence of SEQ ID NO:124 was allowed to bind to the PCR product of primer pair 10,
(XI) purA (426) extension primer consisting of a sequence of SEQ ID NO:127 was allowed to bind to the PCR product of primer pair 11,
(XII) lexA (384) extension primer consisting of a sequence of SEQ ID NO:130 was allowed to bind to the PCR product of primer pair 12, and
(XIII) rpoH (279) extension primer consisting of a sequence of SEQ ID NO:133 was allowed to bind to the PCR product of primer pair 13.

Extension primers were elongated at the 3′-end by exactly one nucleotide. The elongated extension primers were analyzed by mass spectrometry and the nucleotide that has been added to the extension primers was determined.

Information on the phylogenetic origin of the bacterial strains was determined based on a comparison of the pattern of the determined nucleotides with respective predetermined patterns of bacterial strains, which phylogenetic origin was known.

An alignment of the concatenated sequence of determined nucleotides was generated with ClustalW version 2.1 and a phylogenetic tree was generated using default settings and visualized with iTol (http://itol.embl.de/). The resulting tree is shown in FIG. 10. The tree is based only on the phylogeny markers (SNP) including information about presence and absence of genes. As compared to example 4, testing the 8 phylogenetic markers results in an improved resolution of the phylogenetic analysis. However, also with the phylogenetic markers the results were not optimal. In particular, several distinct isolates were not distinguishable.

### Example 6

In a final experiment, a combined analysis utilizing information from both the 13 universal resistance markers and the 8 phylogenetic markers was performed. 55 selected sequenced E. coli isolates were analyzed as described in examples 4 and 5. An alignment of the concatenated sequence of determined nucleotides was generated with ClustalW version 2.1 and a phylogenetic tree was generated using default settings and visualized with iTol (http://itol.embl.de/). The resulting tree is shown in FIG. 11. The tree is based on both the resistance markers and the phylogeny markers including information about presence and absence of genes. The data show that combined testing of the 13 resistance markers and the 8 phylogenetic markers results in further improvement of the resolution of the phylogenetic analysis as compared to the results of example 5.

### DESCRIPTION OF FIGURES

FIG. 1 shows exemplarily binding regions of amplification primers and extension primers in the coding sequences of the antibiotic resistance genes. Binding regions of amplification primers are shown underlined. Binding regions of extension primers are shown in bold letters. Nucleotides of interest are in [brackets].

FIG. 1A shows the KPC-2 coding sequence of SEQ ID NO:87 and exemplary primer binding regions and nucleotides of interest.

FIG. 1B shows the NDM-1 coding sequence of SEQ ID NO:88 and exemplary primer binding regions and nucleotides of interest.

FIG. 1C shows the OXA-9 coding sequence of SEQ ID NO:89 and exemplary primer binding regions and nucleotides of interest.

FIG. 1D shows the OXA-48 coding sequence of SEQ ID NO:90 and exemplary primer binding regions and nucleotides of interest.

FIG. 1E shows the CTX-M-9 coding sequence of SEQ ID NO:91 and exemplary primer binding regions and nucleotides of interest.

FIG. 1F shows the CTX-M-15 coding sequence of SEQ ID NO:92 and exemplary primer binding regions and nucleotides of interest.

FIG. 1G shows the AAC(6′)-lb-cr coding sequence of SEQ ID NO:93 and exemplary primer binding regions and nucleotides of interest. Binding regions of two pairs of amplification primers and of two corresponding extension primers are shown.

FIG. 2 shows exemplarily binding regions of amplification primers and extension primers in the coding sequences of the phylogenetic Klebsiella markers. Binding regions of amplification primers are shown underlined. Binding regions of extension primers are shown in bold letters. Nucleotides of interest are in [brackets].

FIG. 2A shows the gapA coding sequence of SEQ ID NO:28 and exemplary primer binding regions and nucleotides of interest. Binding regions of two pairs of amplification primers and of two corresponding extension primers are shown.

FIG. 2B shows residues 1 to 1852 of the infB coding sequence of SEQ ID NO:29 and exemplary primer binding regions and nucleotides of interest.

FIG. 2C shows the mdh coding sequence of SEQ ID NO:70 and exemplary primer binding regions and nucleotides of interest.

FIG. 2D shows the phoE coding sequence of SEQ ID NO:71 and exemplary primer binding regions and nucleotides of interest. Binding regions of two pairs of amplification primers and of two corresponding extension primers are shown. For better distinguishability the exemplary binding regions of one of the primer pairs are shown with increased font size. The double underlined residue is part of two exemplary primer binding regions.

FIG. 2E shows residues 1465 to 2131 of the rpoB coding sequence of SEQ ID NO:94 and exemplary primer binding regions and nucleotides of interest.

FIG. 3 shows a phylogenetic tree generated in MEGA6 using Neighbor Joining without bootstrapping. The scale bar shows the length of branch that represents an evolutionary distance of 1 nucleotide per position in the sequence. The tree is based only on the resistance markers (SNP) including information about presence and absence of genes. Identifiers of the 93 sequenced K. pneumoniae isolates are shown in the surrounding. The lines in the center indicate the phylogenetic grouping based on the results of experiment 1.

FIG. 4 shows a phylogenetic tree generated in MEGA6 using Neighbor Joining without bootstrapping. The scale bar shows the length of branch that represents an evolutionary distance of 0.1 nucleotides per position in the sequence. The tree is based only on the on the phylogeny markers (SNP) including information about presence and absence of genes. Identifiers of the 93 sequenced K. pneumoniae isolates are shown in the surrounding. The lines in the center indicate the phylogenetic grouping based on the results of experiment 2.

FIG. 5 shows a phylogenetic tree generated in MEGA6 using Neighbor Joining without bootstrapping. The scale bar shows the length of branch that represents an evolutionary distance of 0.1 nucleotides per position in the sequence. The tree is based on both the resistance markers and the phylogeny markers including information about presence and absence of genes. Identifiers of the 93 sequenced K. pneumoniae isolates are shown in the surrounding. The lines in the center indicate the phylogenetic grouping based on the results of experiment 3.

FIG. 6 shows exemplarily binding regions of amplification primers and extension primers in the coding sequences of further antibiotic resistance genes. Binding regions of amplification primers are shown underlined. Binding regions of extension primers are shown in bold letters. Nucleotides of interest are in [brackets].

FIG. 6A shows the OXA-1 coding sequence of SEQ ID NO:163 and exemplary primer binding regions and nucleotides of interest.

FIG. 6B shows the OXA-2 coding sequence of SEQ ID NO:164 and exemplary primer binding regions and nucleotides of interest.

FIG. 6C shows the TEM-1 coding sequence of SEQ ID NO:165 and exemplary primer binding regions and nucleotides of interest.

FIG. 6D shows the APHA3-la coding sequence of SEQ ID NO:166 and exemplary primer binding regions and nucleotides of interest.

FIG. 6E shows the AAC(3′)-IIae coding sequence of SEQ ID NO:167 and exemplary primer binding regions and nucleotides of interest.

FIG. 7 shows exemplarily binding regions of amplification primers and extension primers in the coding sequences of the Escherichia resistance markers used for phylogenetic evaluation. Binding regions of amplification primers are shown underlined. Binding regions of extension primers are shown in bold letters. Nucleotides of interest are in [brackets].

FIG. 7A shows the gyrB coding sequence of SEQ ID NO:168 and exemplary primer binding regions and nucleotides of interest.

FIG. 7B shows the adk coding sequence of SEQ ID NO:169 and exemplary primer binding regions and nucleotides of interest. Binding regions of two pairs of amplification primers and of two corresponding extension primers are shown. For better distinguishability the exemplary binding regions of the second pair of amplification primers and of the corresponding extension primer are shown in italics.

FIG. 7C shows the icd coding sequence of SEQ ID NO:170 and exemplary primer binding regions and nucleotides of interest. Binding regions of two pairs of amplification primers and of two corresponding extension primers are shown. For better distinguishability the exemplary binding regions of the second pair of amplification primers and of the corresponding extension primer are shown in italics.

FIG. 7D shows the purA coding sequence of SEQ ID NO:171 and exemplary primer binding regions and nucleotides of interest.

FIG. 7E shows the lexA coding sequence of SEQ ID NO:172 and exemplary primer binding regions and nucleotides of interest.

FIG. 7F shows the rpoH coding sequence of SEQ ID NO:173 and exemplary primer binding regions and nucleotides of interest.

FIG. 8 shows a phylogenetic tree based on an alignment generated with ClustalW version 2.1 using default settings and visualized with iTol (http://itol.embl.de/). The scale bar shows the length of branch that represents an evolutionary distance of 0.1 nucleotide per position in the sequence. The tree is based only on 8 resistance markers (SNP) including information about presence and absence of genes. Identifiers of 55 sequenced E. coli isolates are shown in the surrounding. The lines in the center indicate the phylogenetic grouping based on the results of experiment 4.

FIG. 9 shows a phylogenetic tree based on an alignment generated with ClustalW version 2.1 using default settings and visualized with iTol (http://itol.embl.de/). The scale bar shows the length of branch that represents an evolutionary distance of 0.1 nucleotide per position in the sequence. The tree is based only on 13 resistance markers (SNP) including information about presence and absence of genes. Identifiers of 55 sequenced E. coli isolates are shown in the surrounding. The lines in the center indicate the phylogenetic grouping based on the results of experiment 4.

FIG. 10 shows a phylogenetic tree based on an alignment generated with ClustalW version 2.1 using default settings and visualized with iTol (http://itol.embl.de/). The scale bar shows the length of branch that represents an evolutionary distance of 0.1 nucleotide per position in the sequence. The tree is based only on 8 E. coli phylogeny markers (SNP) including information about presence and absence of genes. Identifiers of 55 sequenced E. coli isolates are shown in the surrounding. The lines in the center indicate the phylogenetic grouping based on the results of experiment 5.

FIG. 11 shows a phylogenetic tree based on an alignment generated with ClustalW version 2.1 using default settings and visualized with iTol (http://itol.embl.de/). The scale bar shows the length of branch that represents an evolutionary distance of 0.1 nucleotides per position in the sequence. The tree is based on both 13 universal resistance markers and 8 E. coli phylogeny markers including information about presence and absence of genes. Identifiers of 55 sequenced E. coli isolates are shown in the surrounding. The lines in the center indicate the phylogenetic grouping based on the results of experiment 6.

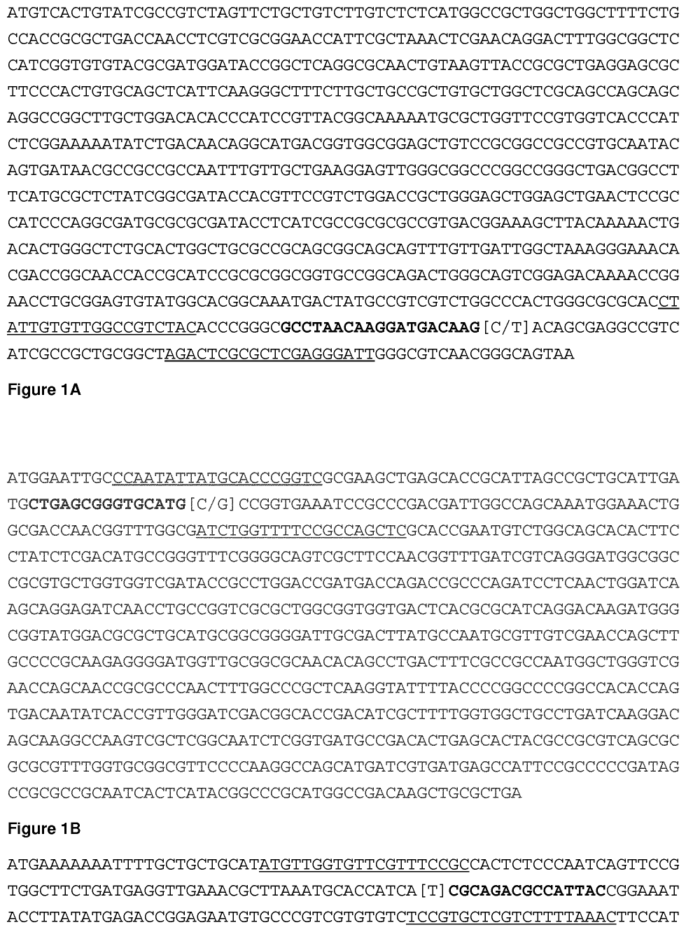

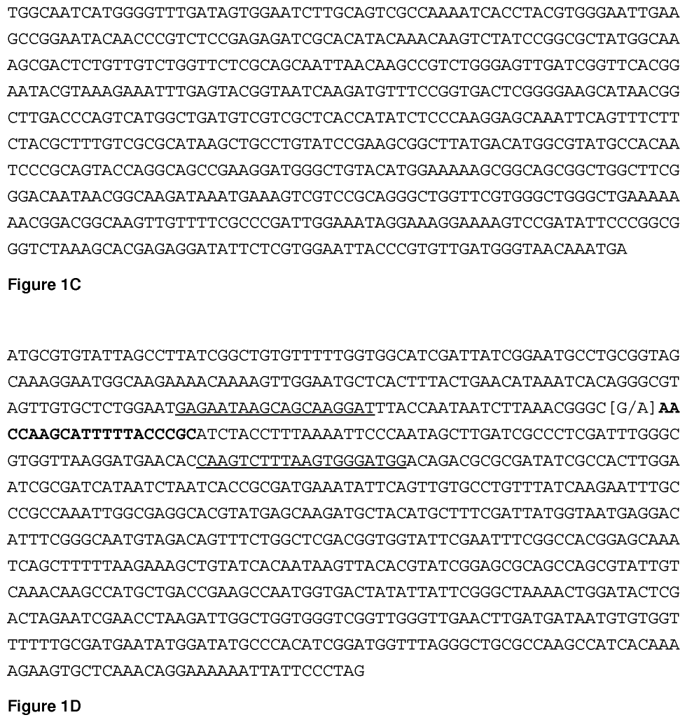

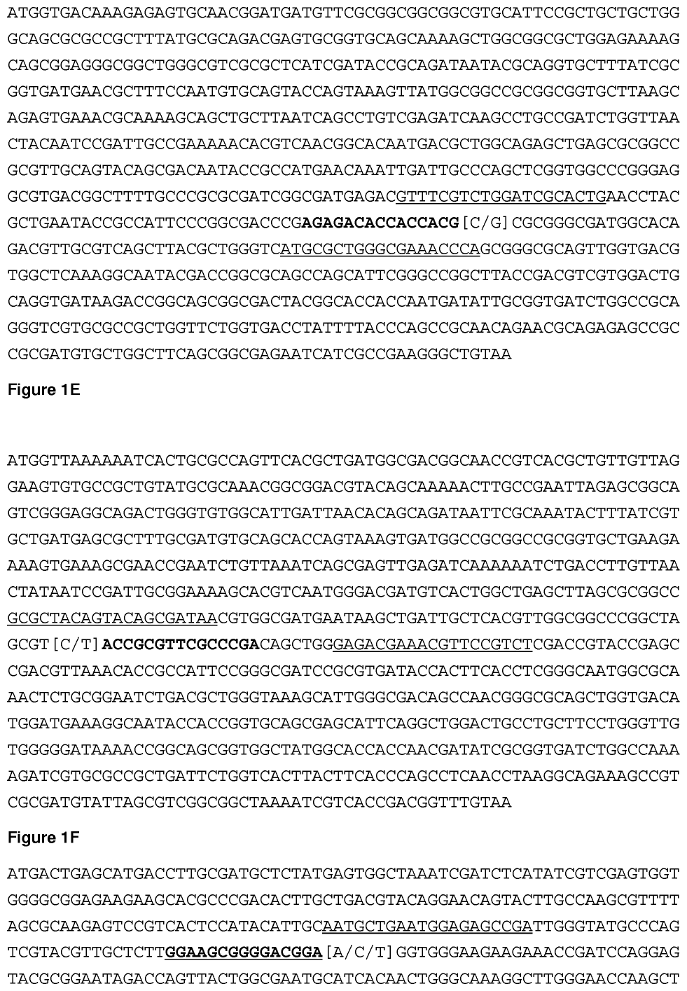

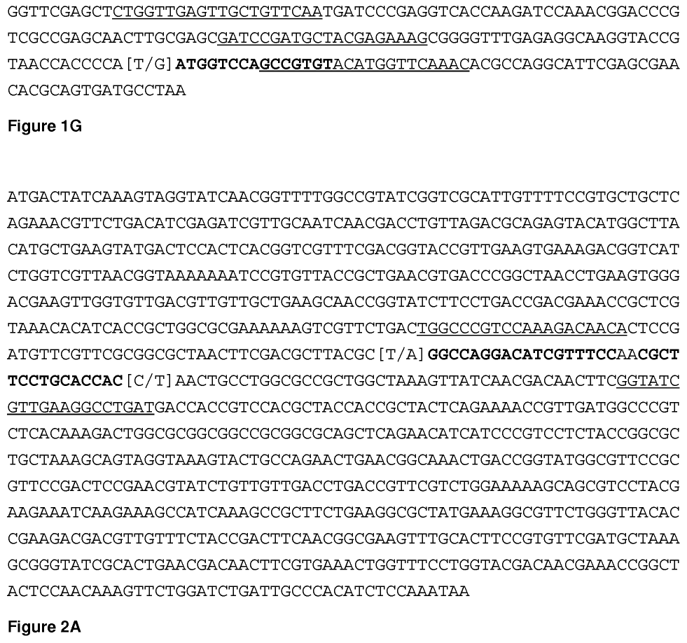

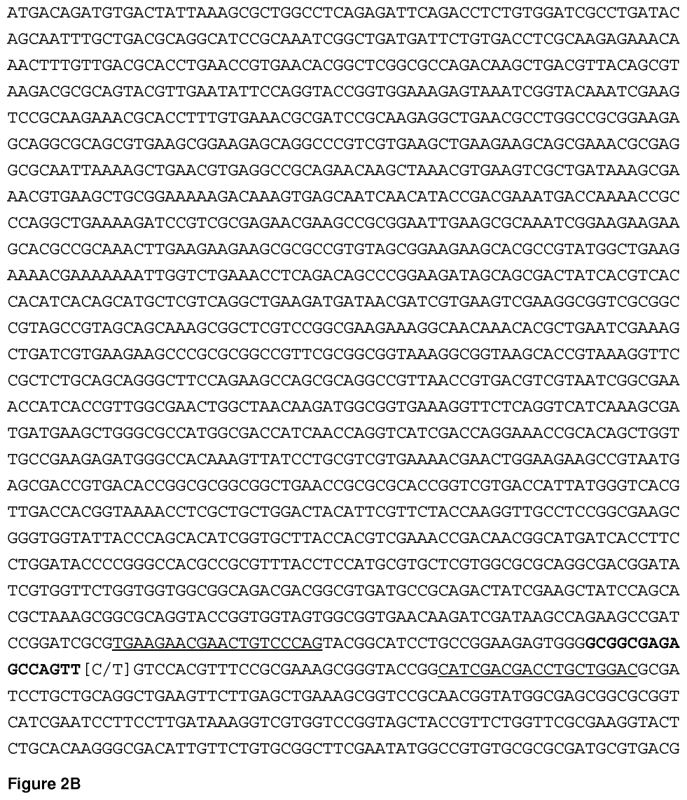

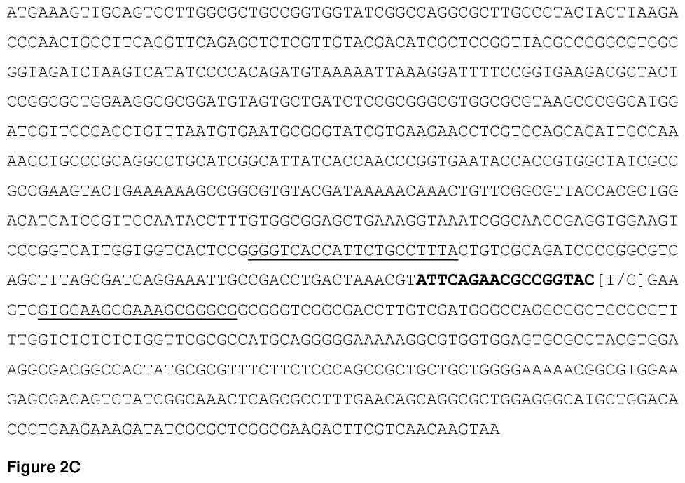

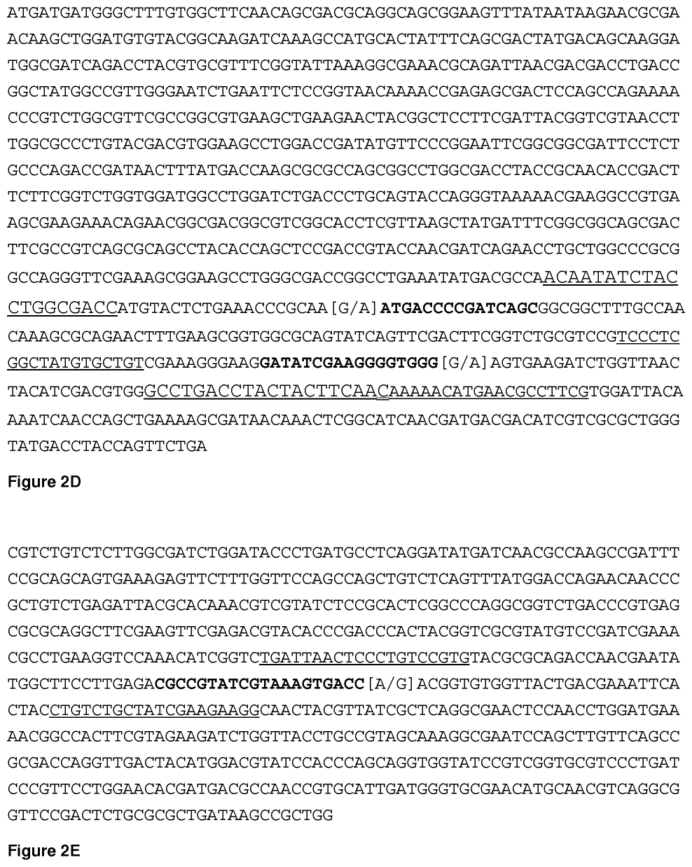

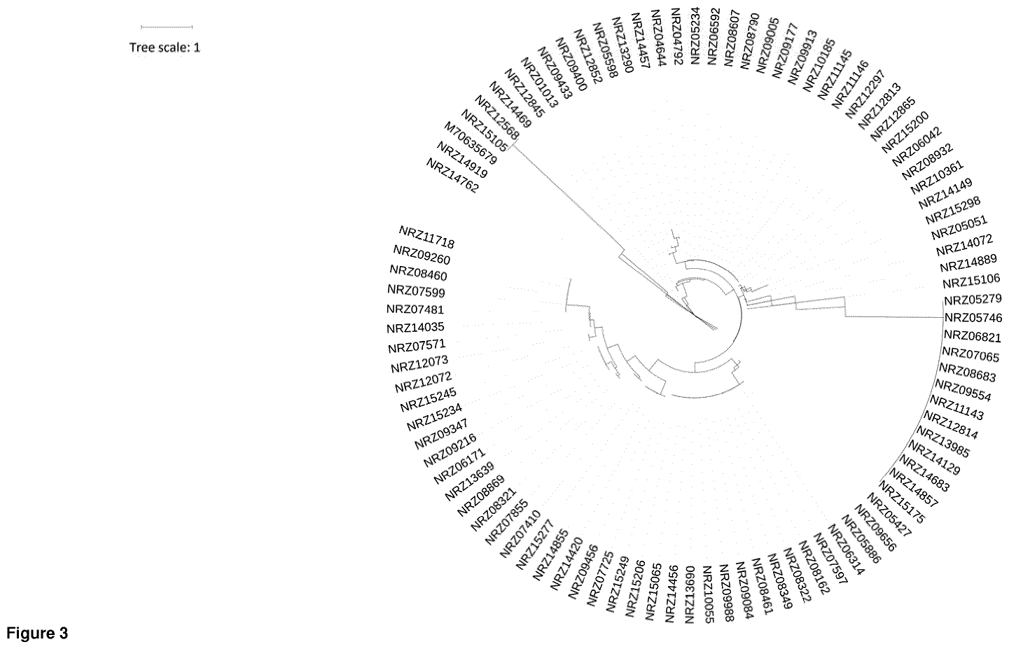

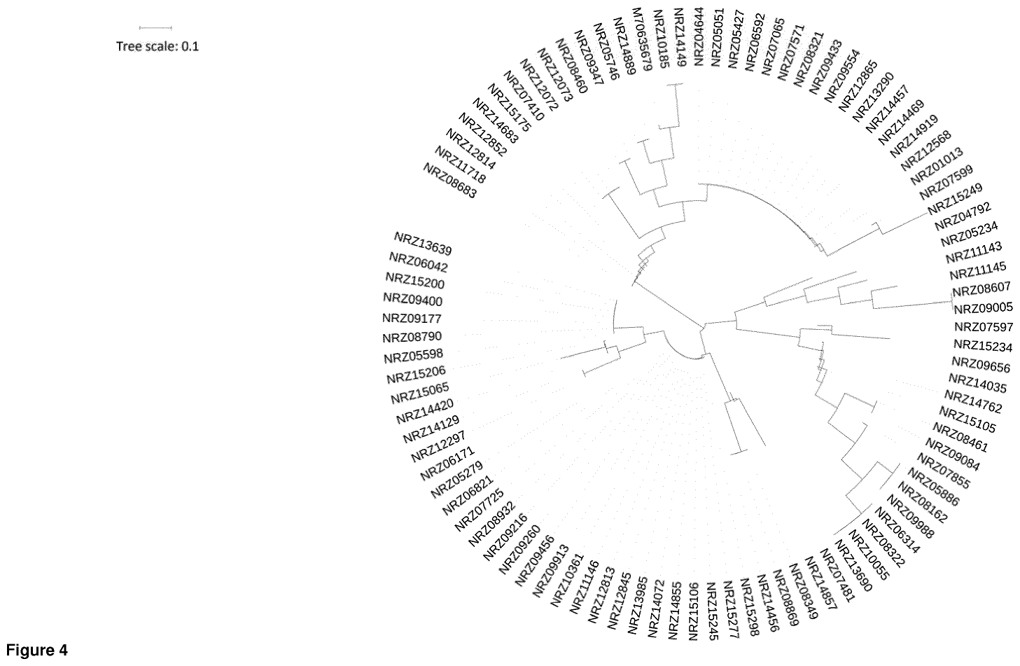

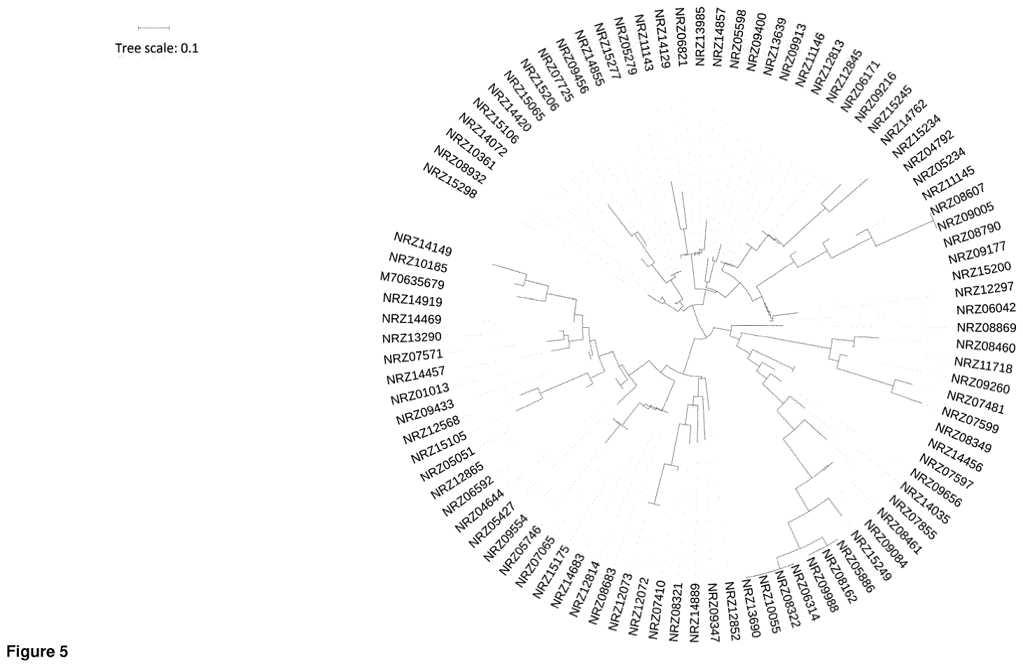

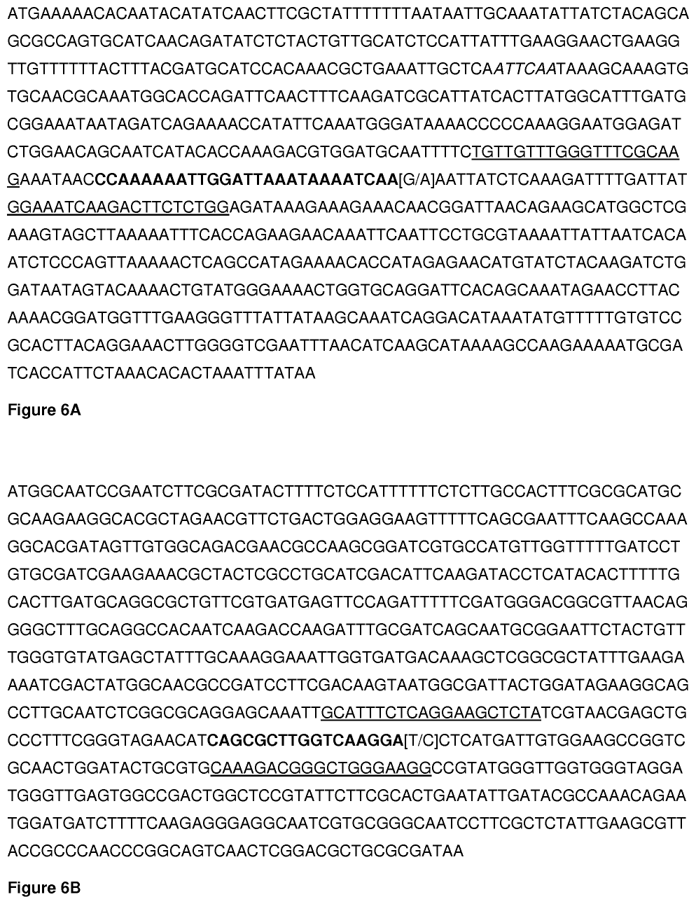

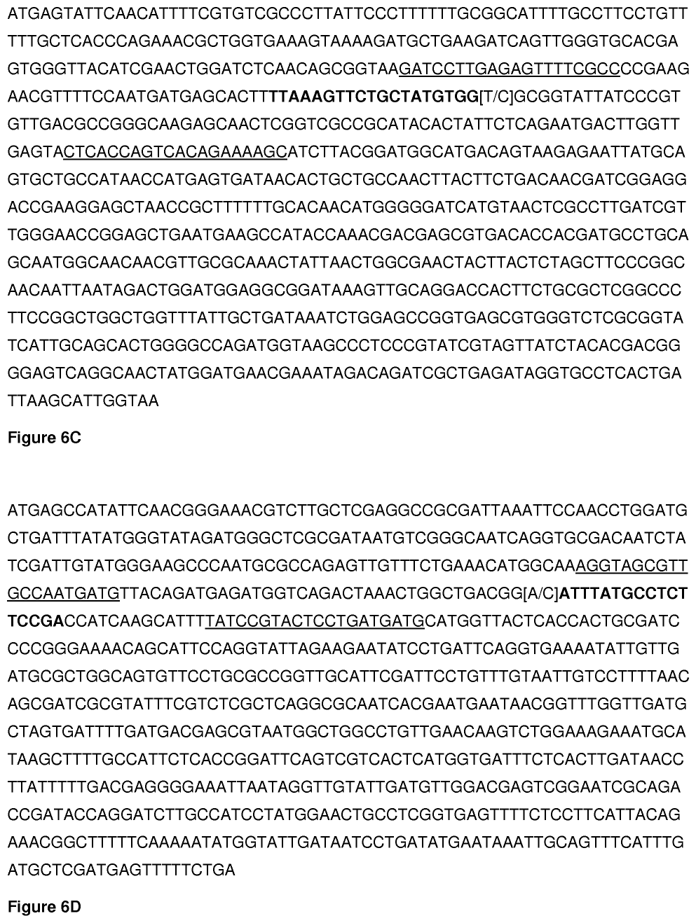

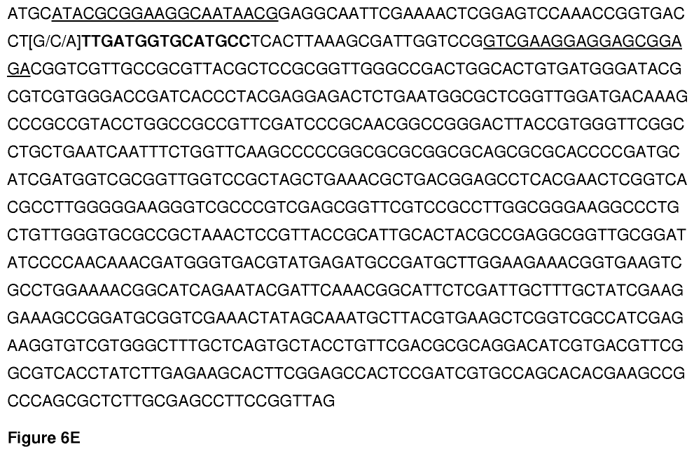

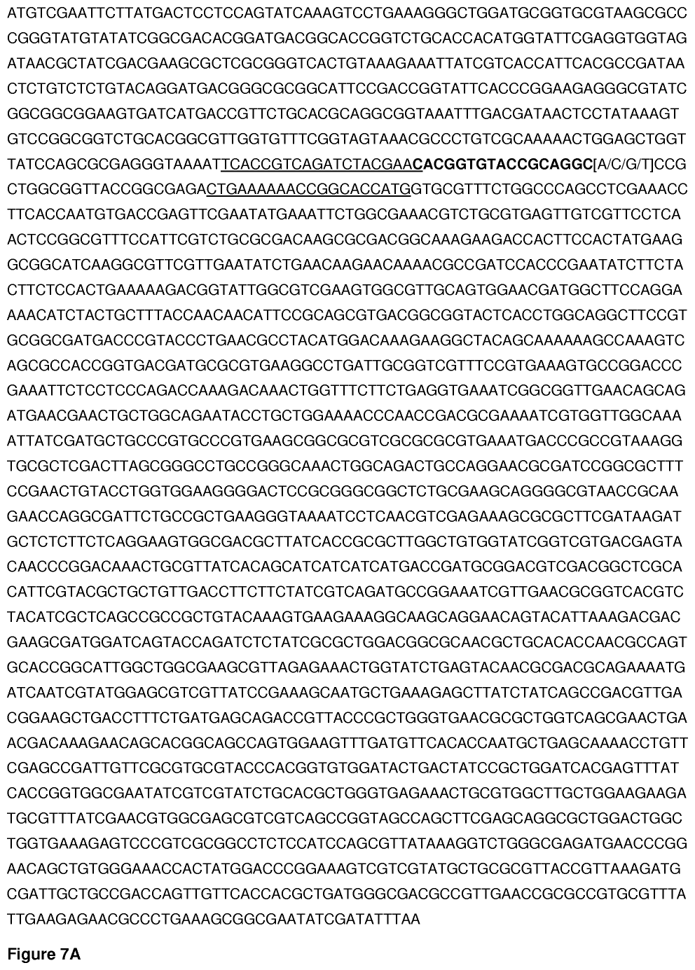

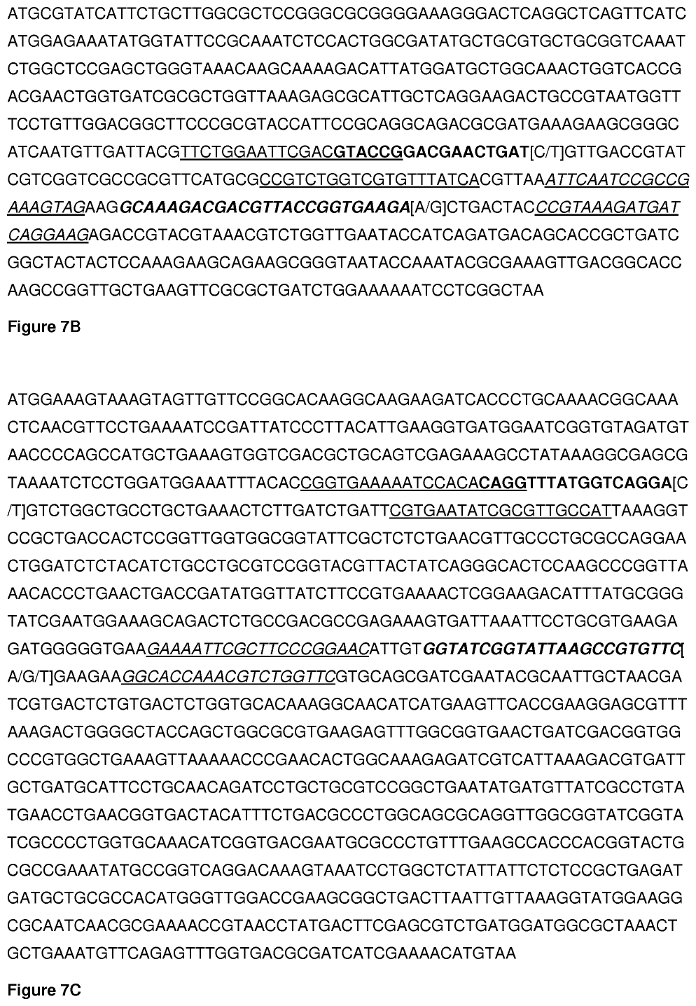

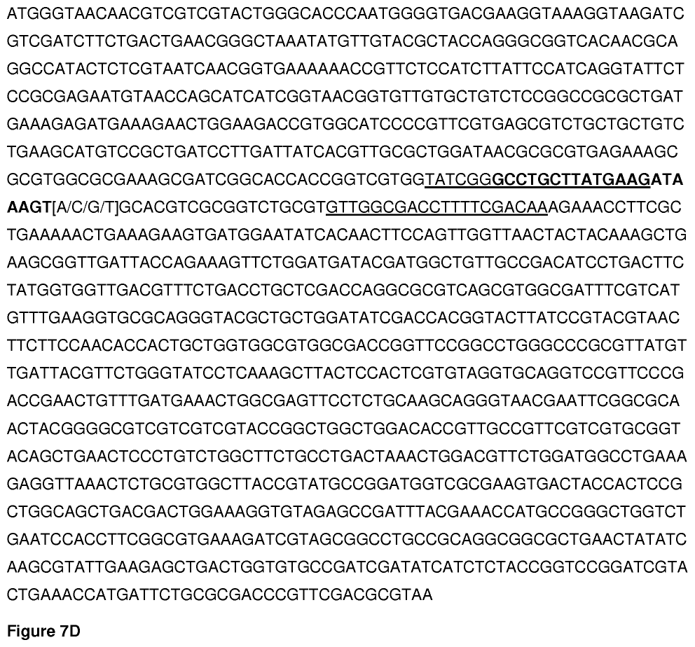

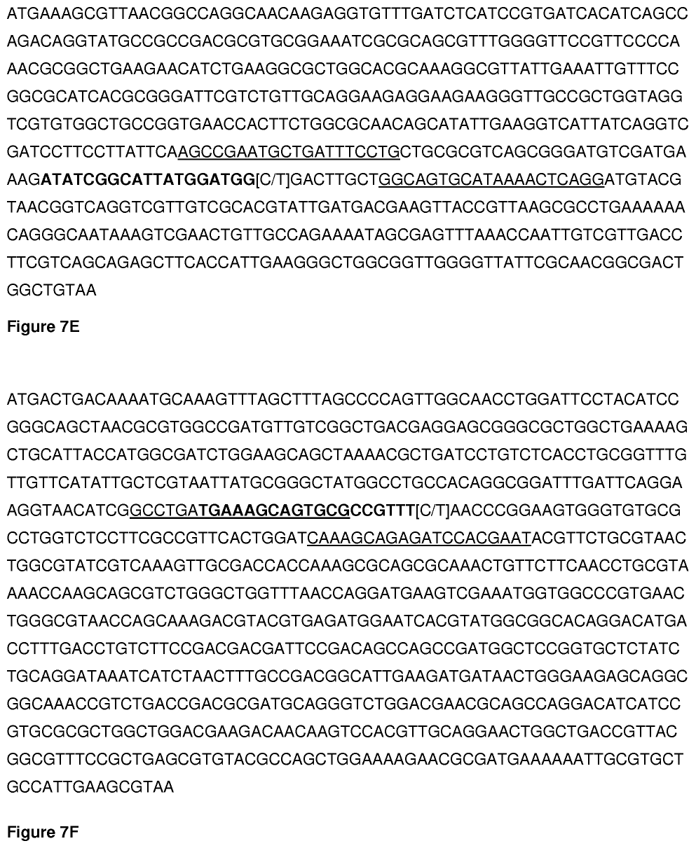

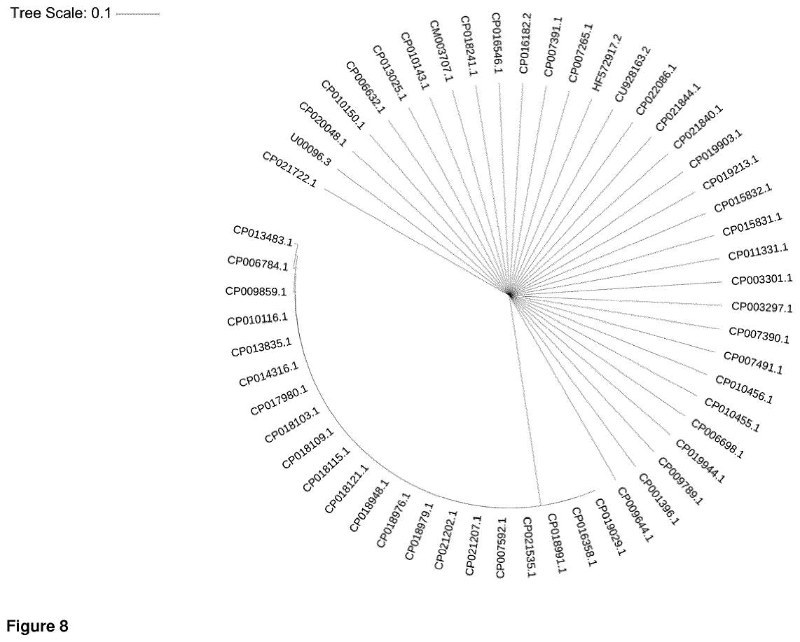

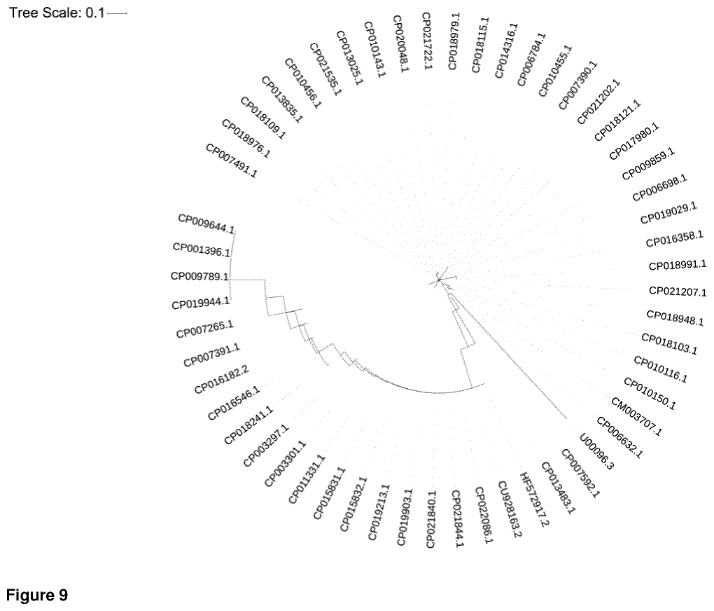

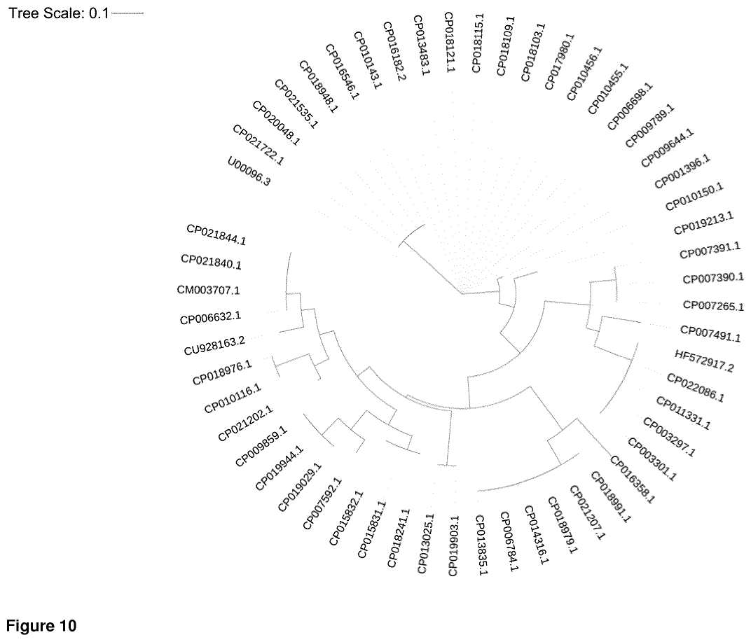

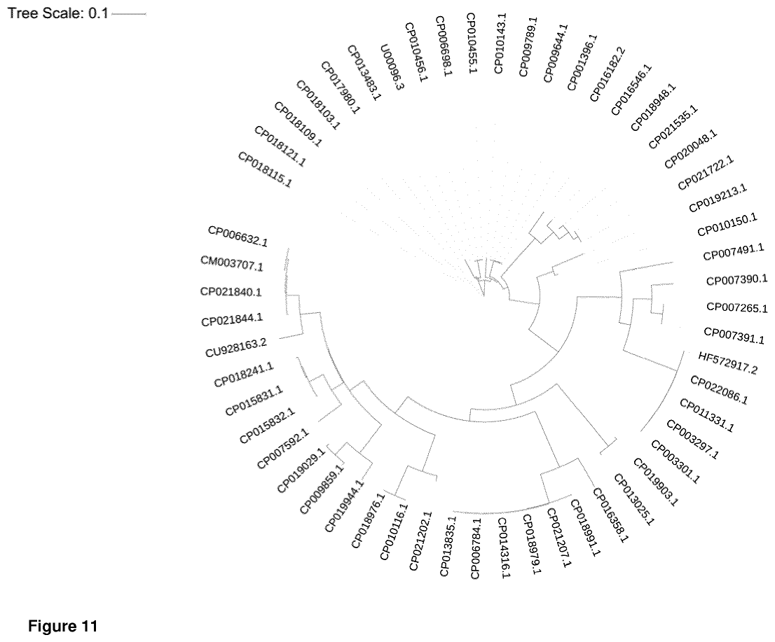

1. A method for determining from a bacterial sample information on both the phylogenetic origin and the antibiotic resistances of bacterial strains, the method comprising the step of:
A) Performing PCR reactions comprising the following primer pairs:
a. Primer pair 1 consisting of a forward primer comprising a sequence of SEQ ID NO:46 or a sequence having at least 80% sequence identity therewith, and a reverse primer comprising a sequence of SEQ ID NO:47 or a sequence having at least 80% sequence identity therewith,
b. Primer pair 2 consisting of a forward primer comprising a sequence of SEQ ID NO:48 or a sequence having at least 80% sequence identity therewith, and a reverse primer comprising a sequence of SEQ ID NO:49 or a sequence having at least 80% sequence identity therewith,
c. Primer pair 3 consisting of a forward primer comprising a sequence of SEQ ID NO:51 or a sequence having at least 80% sequence identity therewith, and a reverse primer comprising a sequence of SEQ ID NO:52 or a sequence having at least 80% sequence identity therewith,
d. Primer pair 4 consisting of a forward primer comprising a sequence of SEQ ID NO:54 or a sequence having at least 80% sequence identity therewith, and a reverse primer comprising a sequence of SEQ ID NO:55 or a sequence having at least 80% sequence identity therewith,
e. Primer pair 5 consisting of a forward primer comprising a sequence of SEQ ID NO:57 or a sequence having at least 80% sequence identity therewith, and a reverse primer comprising a sequence of SEQ ID NO:58 or a sequence having at least 80% sequence identity therewith,
f. Primer pair 6 consisting of a forward primer comprising a sequence of SEQ ID NO:60 or a sequence having at least 80% sequence identity therewith, and a reverse primer comprising a sequence of SEQ ID NO:61 or a sequence having at least 80% sequence identity therewith,
g. Primer pair 7 consisting of a forward primer comprising a sequence of SEQ ID NO:62 or a sequence having at least 80% sequence identity therewith, and a reverse primer comprising a sequence of SEQ ID NO:63 or a sequence having at least 80% sequence identity therewith, and
h. Primer pair 8 consisting of a forward primer comprising a sequence of SEQ ID NO:65 or a sequence having at least 80% sequence identity therewith, and a reverse primer comprising a sequence of SEQ ID NO:66 or a sequence having at least 80% sequence identity therewith.

2. The method according to claim 1 comprising the step of:
A) Performing PCR reactions comprising the following primer pairs:
a. Primer pair 1 consisting of a forward primer comprising a sequence of SEQ ID NO:1 or a sequence having at least 80% sequence identity therewith, and a reverse primer comprising a sequence of SEQ ID NO:2 or a sequence having at least 80% sequence identity therewith,
b. Primer pair 2 consisting of a forward primer comprising a sequence of SEQ ID NO:4 or a sequence having at least 80% sequence identity therewith, and a reverse primer comprising a sequence of SEQ ID NO:5 or a sequence having at least 80% sequence identity therewith,
c. Primer pair 3 consisting of a forward primer comprising a sequence of SEQ ID NO:7 or a sequence having at least 80% sequence identity therewith, and a reverse primer comprising a sequence of SEQ ID NO:8 or a sequence having at least 80% sequence identity therewith,
d. Primer pair 4 consisting of a forward primer comprising a sequence of SEQ ID NO:10 or a sequence having at least 80% sequence identity therewith, and a reverse primer comprising a sequence of SEQ ID NO:11 or a sequence having at least 80% sequence identity therewith,
e. Primer pair 5 consisting of a forward primer comprising a sequence of SEQ ID NO:13 or a sequence having at least 80% sequence identity therewith, and a reverse primer comprising a sequence of SEQ ID NO:14 or a sequence having at least 80% sequence identity therewith,
f. Primer pair 6 consisting of a forward primer comprising a sequence of SEQ ID NO:16 or a sequence having at least 80% sequence identity therewith, and a reverse primer comprising a sequence of SEQ ID NO:17 or a sequence having at least 80% sequence identity therewith,
g. Primer pair 7 consisting of a forward primer comprising a sequence of SEQ ID NO:19 or a sequence having at least 80% sequence identity therewith, and a reverse primer comprising a sequence of SEQ ID NO:20 or a sequence having at least 80% sequence identity therewith, and
h. Primer pair 8 consisting of a forward primer comprising a sequence of SEQ ID NO:22 or a sequence having at least 80% sequence identity therewith, and a reverse primer comprising a sequence of SEQ ID NO:23 or a sequence having at least 80% sequence identity therewith.

3. The method according to claim 1 comprising the step of:
A) Performing PCR reactions comprising one or more of the following additional primer pairs:
(I) Primer pair 9 consisting of a forward primer comprising a sequence of SEQ ID NO:134 or a sequence having at least 80% sequence identity therewith, and a reverse primer comprising a sequence of SEQ ID NO:135 or a sequence having at least 80% sequence identity therewith,
(II) Primer pair 10 consisting of a forward primer comprising a sequence of SEQ ID NO:136 or a sequence having at least 80% sequence identity therewith, and a reverse primer comprising a sequence of SEQ ID NO:137 or a sequence having at least 80% sequence identity therewith,
(III) Primer pair 11 consisting of a forward primer comprising a sequence of SEQ ID NO:138 or a sequence having at least 80% sequence identity therewith, and a reverse primer comprising a sequence of SEQ ID NO:139 or a sequence having at least 80% sequence identity therewith,
(IV) Primer pair 12 consisting of a forward primer comprising a sequence of SEQ ID NO:141 or a sequence having at least 80% sequence identity therewith, and a reverse primer comprising a sequence of SEQ ID NO:142 or a sequence having at least 80% sequence identity therewith,
(V) Primer pair 13 consisting of a forward primer comprising a sequence of SEQ ID NO:144 or a sequence having at least 80% sequence identity therewith, and a reverse primer comprising a sequence of SEQ ID NO:145 or a sequence having at least 80% sequence identity therewith.

4. The method according to claim 3 comprising the step of:
A) Performing PCR reactions comprising one or more of the following primer pairs:
(I) Primer pair 9 consisting of a forward primer comprising a sequence of SEQ ID NO:95 or a sequence having at least 80% sequence identity therewith, and a reverse primer comprising a sequence of SEQ ID NO:96 or a sequence having at least 80% sequence identity therewith,
(II) Primer pair 10 consisting of a forward primer comprising a sequence of SEQ ID NO:98 or a sequence having at least 80% sequence identity therewith, and a reverse primer comprising a sequence of SEQ ID NO:99 or a sequence having at least 80% sequence identity therewith,
(III) Primer pair 11 consisting of a forward primer comprising a sequence of SEQ ID NO:101 or a sequence having at least 80% sequence identity therewith, and a reverse primer comprising a sequence of SEQ ID NO:102 or a sequence having at least 80% sequence identity therewith,
(IV) Primer pair 12 consisting of a forward primer comprising a sequence of SEQ ID NO:104 or a sequence having at least 80% sequence identity therewith, and a reverse primer comprising a sequence of SEQ ID NO:105 or a sequence having at least 80% sequence identity therewith,
(V) Primer pair 13 consisting of a forward primer comprising a sequence of SEQ ID NO:107 or a sequence having at least 80% sequence identity therewith, and a reverse primer comprising a sequence of SEQ ID NO:108 or a sequence having at least 80% sequence identity therewith.

5. The method according to claim 1 comprising the additional step of:
B) Allowing extension primers to bind to the PCR products obtained from the PCR reactions of step A).

6. The method according to claim 5, wherein at least one extension primer is binding to each of the PCR products obtained from step A).

7. The method according to claim 5, wherein the following extension primers are allowed to bind to the PCR products obtained from the PCR reactions of step A):
a. KPC-2 extension primer comprising a sequence of SEQ ID NO:3 or a sequence having a sequence identity of at least 80% with SEQ ID NO:3,
b. NDM-1 extension primer comprising a sequence of SEQ ID NO:50 or a sequence having a sequence identity of at least 80% with SEQ ID NO:50,
c. OXA-9 extension primer comprising a sequence of SEQ ID NO:53 or a sequence having a sequence identity of at least 80% with SEQ ID NO:53,
d. OXA-48 extension primer comprising a sequence of SEQ ID NO:56 or a sequence having a sequence identity of at least 80% with SEQ ID NO:56,
e. CTX-M-9 extension primer comprising a sequence of SEQ ID NO:59 or a sequence having a sequence identity of at least 80% with SEQ ID NO:59,
f. CTX-M-15 extension primer comprising a sequence of SEQ ID NO:18 or a sequence having a sequence identity of at least 80% with SEQ ID NO:18,
g. AAC(6′)-lb-cr (223) extension primer comprising a sequence of SEQ ID NO:64 or a sequence having a sequence identity of at least 80% with SEQ ID NO:64, and
h. AAC(6′)-lb-cr (454) extension primer comprising a sequence of SEQ ID NO:24 or a sequence having a sequence identity of at least 80% with SEQ ID NO:24.

8. The method according to claim 5, wherein following extension primers are allowed to bind to the PCR products obtained from the PCR reactions of step A):
a. KPC-2 extension primer comprising a sequence of SEQ ID NO:3 or a sequence having a sequence identity of at least 80% with SEQ ID NO:3,
b. NDM-1 extension primer comprising a sequence of SEQ ID NO:6 or a sequence having a sequence identity of at least 80% with SEQ ID NO:6,
c. OXA-9 extension primer comprising a sequence of SEQ ID NO:9 or a sequence having a sequence identity of at least 80% with SEQ ID NO:9,
d. OXA-48 extension primer comprising a sequence of SEQ ID NO:12 or a sequence having a sequence identity of at least 80% with SEQ ID NO:12,
e. CTX-M-9 extension primer comprising a sequence of SEQ ID NO:15 or a sequence having a sequence identity of at least 80% with SEQ ID NO:15,
f. CTX-M-15 extension primer comprising a sequence of SEQ ID NO:18 or a sequence having a sequence identity of at least 80% with SEQ ID NO:18,
g. AAC(6′)-lb-cr (223) extension primer comprising a sequence of SEQ ID NO:21 or a sequence having a sequence identity of at least 80% with SEQ ID NO:21, and
h. AAC(6′)-lb-cr (454) extension primer comprising a sequence of SEQ ID NO:24 or a sequence having a sequence identity of at least 80% with SEQ ID NO:24.

9. The method according to claim 7, wherein one or more of the following additional extension primers are allowed to bind to the PCR products obtained from the PCR reactions of step A):
(I) OXA-1 extension primer comprising a sequence of SEQ ID NO:97 or a sequence having a sequence identity of at least 80% with SEQ ID NO:97,
(II) OXA-2 extension primer comprising a sequence of SEQ ID NO:100 or a sequence having a sequence identity of at least 80% with SEQ ID NO:100,
(III) TEM-1 extension primer comprising a sequence of SEQ ID NO:140 or a sequence having a sequence identity of at least 80% with SEQ ID NO:140,
(IV) APHA3-Ia extension primer comprising a sequence of SEQ ID NO:143 or a sequence having a sequence identity of at least 80% with SEQ ID NO:143,
(V) AAC(3′)-IIae extension primer comprising a sequence of SEQ ID NO:109 or a sequence having a sequence identity of at least 80% with SEQ ID NO:109.

10. The method according to claim 9, wherein one or more of the following extension primers are allowed to bind to the PCR products obtained from the PCR reactions of step A):
(I) OXA-1 extension primer comprising a sequence of SEQ ID NO:97 or a sequence having a sequence identity of at least 80% with SEQ ID NO:97,
(II) OXA-2 extension primer comprising a sequence of SEQ ID NO:100 or a sequence having a sequence identity of at least 80% with SEQ ID NO:100,
(III) TEM-1 extension primer comprising a sequence of SEQ ID NO:103 or a sequence having a sequence identity of at least 80% with SEQ ID NO:103,
(IV) APHA3-Ia extension primer comprising a sequence of SEQ ID NO:106 or a sequence having a sequence identity of at least 80% with SEQ ID NO:106,
(V) AAC(3′)-IIae extension primer comprising a sequence of SEQ ID NO:109 or a sequence having a sequence identity of at least 80% with SEQ ID NO:109.

11. The method according to claim 1, wherein the PCR reactions of step A) additionally comprise at least one of the following primer pairs:
i. Primer pair 14 consisting of a forward primer comprising a sequence of SEQ ID NO:67 or a sequence having at least 80% sequence identity therewith, and a reverse primer comprising a sequence of SEQ ID NO:68 or a sequence having at least 80% sequence identity therewith,
j. Primer pair 15 consisting of a forward primer comprising a sequence of SEQ ID NO:73 or a sequence having at least 80% sequence identity therewith, and a reverse primer comprising a sequence of SEQ ID NO:74 or a sequence having at least 80% sequence identity therewith,
k. Primer pair 16 consisting of a forward primer comprising a sequence of SEQ ID NO:76 or a sequence having at least 80% sequence identity therewith, and a reverse primer comprising a sequence of SEQ ID NO:77 or a sequence having at least 80% sequence identity therewith,
l. Primer pair 17 consisting of a forward primer comprising a sequence of SEQ ID NO:79 or a sequence having at least 80% sequence identity therewith, and a reverse primer comprising a sequence of SEQ ID NO:80 or a sequence having at least 80% sequence identity therewith,
m. Primer pair 18 consisting of a forward primer comprising a sequence of SEQ ID NO:82 or a sequence having at least 80% sequence identity therewith, and a reverse primer comprising a sequence of SEQ ID NO:83 or a sequence having at least 80% sequence identity therewith, and
n. Primer pair 19 consisting of a forward primer comprising a sequence of SEQ ID NO:84 or a sequence having at least 80% sequence identity therewith, and a reverse primer comprising a sequence of SEQ ID NO:85 or a sequence having at least 80% sequence identity therewith.

12. The method according to claim 11, wherein additionally at least one of the following extension primers is allowed to bind to the PCR products obtained from the PCR reactions of step A):
i. gapA (420) extension primer comprising a sequence of SEQ ID NO:69 or a sequence having a sequence identity of at least 80% with SEQ ID NO:69,
j. gapA (456) extension primer comprising a sequence of SEQ ID NO:72 or a sequence having a sequence identity of at least 80% with SEQ ID NO:72,
k. infB (1608) extension primer comprising a sequence of SEQ ID NO:75 or a sequence having a sequence identity of at least 80% with SEQ ID NO:75,
l. mdh (633) extension primer comprising a sequence of SEQ ID NO:78 or a sequence having a sequence identity of at least 80% with SEQ ID NO:78,
m. phoE (732) extension primer comprising a sequence of SEQ ID NO:81 or a sequence having a sequence identity of at least 80% with SEQ ID NO:81,
n. phoE (867) extension primer comprising a sequence of SEQ ID NO:42 or a sequence having a sequence identity of at least 80% with SEQ ID NO:42, and
o. rpoB (1819) extension primer comprising a sequence of SEQ ID NO:86 or a sequence having a sequence identity of at least 80% with SEQ ID NO:86.

13. The method according to claim 1, wherein the PCR reactions of step A) additionally comprise at least one of the following primer pairs:
(VI) Primer pair 20 consisting of a forward primer comprising a sequence of SEQ ID NO:146 or a sequence having at least 80% sequence identity therewith, and a reverse primer comprising a sequence of SEQ ID NO:147 or a sequence having at least 80% sequence identity therewith,
(VII) Primer pair 21 consisting of a forward primer comprising a sequence of SEQ ID NO:149 or a sequence having at least 80% sequence identity therewith, and a reverse primer comprising a sequence of SEQ ID NO:150 or a sequence having at least 80% sequence identity therewith,
(VIII) Primer pair 22 consisting of a forward primer comprising a sequence of SEQ ID NO:151 or a sequence having at least 80% sequence identity therewith, and a reverse primer comprising a sequence of SEQ ID NO:152 or a sequence having at least 80% sequence identity therewith,
(IX) Primer pair 23 consisting of a forward primer comprising a sequence of SEQ ID NO:153 or a sequence having at least 80% sequence identity therewith, and a reverse primer comprising a sequence of SEQ ID NO:154 or a sequence having at least 80% sequence identity therewith,
(X) Primer pair 24 consisting of a forward primer comprising a sequence of SEQ ID NO:155 or a sequence having at least 80% sequence identity therewith, and a reverse primer comprising a sequence of SEQ ID NO:156 or a sequence having at least 80% sequence identity therewith,
(XI) Primer pair 25 consisting of a forward primer comprising a sequence of SEQ ID NO:157 or a sequence having at least 80% sequence identity therewith, and a reverse primer comprising a sequence of SEQ ID NO:158 or a sequence having at least 80% sequence identity therewith,
(XII) Primer pair 26 consisting of a forward primer comprising a sequence of SEQ ID NO:159 or a sequence having at least 80% sequence identity therewith, and a reverse primer comprising a sequence of SEQ ID NO:160 or a sequence having at least 80% sequence identity therewith,
(XIII) Primer pair 27 consisting of a forward primer comprising a sequence of SEQ ID NO:161 or a sequence having at least 80% sequence identity therewith, and a reverse primer comprising a sequence of SEQ ID NO:162 or a sequence having at least 80% sequence identity therewith.

14. The method according to claim 13, wherein additionally at least one of the following extension primers is allowed to bind to the PCR products obtained from the PCR reactions of step A):
(VI) gyrB extension primer comprising a sequence of SEQ ID NO:148 or a sequence having a sequence identity of at least 80% with SEQ ID NO:148,
(VII) adk (348) extension primer comprising a sequence of SEQ ID NO:115 or a sequence having a sequence identity of at least 80% with SEQ ID NO:115,
(VIII) adk (456) extension primer comprising a sequence of SEQ ID NO:118 or a sequence having a sequence identity of at least 80% with SEQ ID NO:118,
(IX) icd (243) extension primer comprising a sequence of SEQ ID NO:121 or a sequence having a sequence identity of at least 80% with SEQ ID NO:121,
(X) icd (606) extension primer comprising a sequence of SEQ ID NO:124 or a sequence having a sequence identity of at least 80% with SEQ ID NO:124,
(XI) purA extension primer comprising a sequence of SEQ ID NO:127 or a sequence having a sequence identity of at least 80% with SEQ ID NO:127,
(XII) lexA extension primer comprising a sequence of SEQ ID NO:130 or a sequence having a sequence identity of at least 80% with SEQ ID NO:130,
(XIII) rpoH extension primer comprising a sequence of SEQ ID NO:133 or a sequence having a sequence identity of at least 80% with SEQ ID NO:133.

15. The method according to claim 1, wherein the bacterial strain is a multi-drug resistant gram-negative bacterium.

16. The method according to claim 1 comprising the additional step of:
C) Elongating the extension primers at the 3′-end by at least one nucleotide.

17. The method according to claim 16 comprising the additional step of:
D) Identifying the at least one nucleotide that has been added to the extension primer.

18. The method according to claim 1 comprising the additional step of:
E) Determining information on both the phylogenetic origin and antibiotic resistances of the bacterial strain based on a comparison of the pattern of the determined nucleotides with respective predetermined patterns of bacterial strains, which phylogenetic origin and antibiotic resistances are known.

19. A kit comprising the following amplification primers:
a. A forward primer comprising a sequence of SEQ ID NO:46 or a sequence having at least 80% sequence identity therewith, and a reverse primer comprising a sequence of SEQ ID NO:47 or a sequence having at least 80% sequence identity therewith,
b. A forward primer comprising a sequence of SEQ ID NO:48 or a sequence having at least 80% sequence identity therewith, and a reverse primer comprising a sequence of SEQ ID NO:49 or a sequence having at least 80% sequence identity therewith,
c. A forward primer comprising a sequence of SEQ ID NO:51 or a sequence having at least 80% sequence identity therewith, and a reverse primer comprising a sequence of SEQ ID NO:52 or a sequence having at least 80% sequence identity therewith,
d. A forward primer comprising a sequence of SEQ ID NO:54 or a sequence having at least 80% sequence identity therewith, and a reverse primer comprising a sequence of SEQ ID NO:55 or a sequence having at least 80% sequence identity therewith,
e. A forward primer comprising a sequence of SEQ ID NO:57 or a sequence having at least 80% sequence identity therewith, and a reverse primer comprising a sequence of SEQ ID NO:58 or a sequence having at least 80% sequence identity therewith,
f. A forward primer comprising a sequence of SEQ ID NO:60 or a sequence having at least 80% sequence identity therewith, and a reverse primer comprising a sequence of SEQ ID NO:61 or a sequence having at least 80% sequence identity therewith,
g. A forward primer comprising a sequence of SEQ ID NO:62 or a sequence having at least 80% sequence identity therewith, and a reverse primer comprising a sequence of SEQ ID NO:63 or a sequence having at least 80% sequence identity therewith, and
h. A forward primer comprising a sequence of SEQ ID NO:65 or a sequence having at least 80% sequence identity therewith, and a reverse primer comprising a sequence of SEQ ID NO:66 or a sequence having at least 80% sequence identity therewith.

20. The kit according to claim 19 further comprising the following extension primers:
a. KPC-2 extension primer comprising a sequence of SEQ ID NO:3 or a sequence having a sequence identity of at least 80% with SEQ ID NO:3,
b. NDM-1 extension primer comprising a sequence of SEQ ID NO:50 or a sequence having a sequence identity of at least 80% with SEQ ID NO:50,
c. OXA-9 extension primer comprising a sequence of SEQ ID NO:53 or a sequence having a sequence identity of at least 80% with SEQ ID NO:53,
d. OXA-48 extension primer comprising a sequence of SEQ ID NO:56 or a sequence having a sequence identity of at least 80% with SEQ ID NO:56,
e. CTX-M-9 extension primer comprising a sequence of SEQ ID NO:59 or a sequence having a sequence identity of at least 80% with SEQ ID NO:59,
f. CTX-M-15 extension primer comprising a sequence of SEQ ID NO:18 or a sequence having a sequence identity of at least 80% with SEQ ID NO:18,
g. AAC(6′)-lb-cr (223) extension primer comprising a sequence of SEQ ID NO:64 or a sequence having a sequence identity of at least 80% with SEQ ID NO:64, and
h. AAC(6′)-lb-cr (454) extension primer comprising a sequence of SEQ ID NO:24 or a sequence having a sequence identity of at least 80% with SEQ ID NO:24.

21. A method for determining from a bacterial sample information on both the phylogenetic origin and the antibiotic resistances of bacterial strains, the method comprising the steps of:
A) determining which nucleotide is present
a. at position 814 in the KPC-2 coding sequence (SEQ ID NO:87),
b. at position 82 in the NDM-1 coding sequence (SEQ ID NO:88),
c. at position 104 in the OXA-9 coding sequence (SEQ ID NO:89),
d. at position 186 in the OXA-48 coding sequence (SEQ ID NO:90),
e. at position 556 in the CTX-M-9 coding sequence (SEQ ID NO:91),
f. at position 453 in the CTX-M-15 coding sequence (SEQ ID NO:92),
g. at position 223 in the AAC(6′)-lb-cr coding sequence (SEQ ID NO:93), and
h. at position 454 in the AAC(6′)-lb-cr coding sequence (SEQ ID NO:93),
or at analogous positions in variants or homologous coding sequences having at least 80% sequence identity with the indicated coding sequences, and
B) determining information on both the phylogenetic origin and antibiotic resistances of the bacterial strain based on a comparison of the pattern of the determined nucleotides with respective predetermined patterns of bacterial strains, which phylogenetic origin and antibiotic resistances are known.

22. The method according to claim 21, wherein step A) of the method further comprises determining which nucleotide is present
(I) at position 408 in the OXA-1 coding sequence (SEQ ID NO:163),
(II) at position 585 in the OXA-2 coding sequence (SEQ ID NO:164),
(III) at position 228 in the TEM-1 coding sequence (SEQ ID NO:165),
(IV) at position 230 in the APHA3-Ia coding sequence (SEQ ID NO:166), and/or
(V) at position 63 in the AAC(3′)-IIae coding sequence (SEQ ID NO:167),
or at analogous positions in variants or homologous coding sequences having at least 80% sequence identity with the indicated coding sequences.


SEQUENCE LISTING (173 sequences)

SEQ ID NO: 1 - DNA, 30 bp, Artificial Sequence
  Primer Sequence
acgttggatgctattgtgttggccgtctac

SEQ ID NO: 2 - DNA, 30 bp, Artificial Sequence
  Primer Sequence
acgttggatgaatccctcgagcgcgagtct

SEQ ID NO: 3 - DNA, 19 bp, Artificial Sequence
  Primer Sequence
gcctaacaaggatgacaag

SEQ ID NO: 4 - DNA, 30 bp, Artificial Sequence
  Primer Sequence
acgttggatgccaatattatgcacccggtc

SEQ ID NO: 5 - DNA, 30 bp, Artificial Sequence
  Primer Sequence
acgttggatggagctggcggaaaaccagat

SEQ ID NO: 6 - DNA, 19 bp, Artificial Sequence
  Primer Sequence
gggactgagcgggtgcatg

SEQ ID NO: 7 - DNA, 30 bp, Artificial Sequence
  Primer Sequence
acgttggatgatgttggtgttcgtttccgc

SEQ ID NO: 8 - DNA, 30 bp, Artificial Sequence
  Primer Sequence
acgttggatggtttaaaagacgagcacgga

SEQ ID NO: 9 - DNA, 17 bp, Artificial Sequence
  Primer Sequence
ctgtaatggcgtctgcg

SEQ ID NO: 10 - DNA, 29 bp, Artificial Sequence
  Primer Sequence
acgttggatggagaataagcagcaaggat

SEQ ID NO: 11 - DNA, 30 bp, Artificial Sequence
  Primer Sequence
acgttggatgccatcccacttaaagacttg

SEQ ID NO: 12 - DNA, 22 bp, Artificial Sequence
  Primer Sequence
gggcgggtaaaaatgcttggtt

SEQ ID NO: 13 - DNA, 30 bp, Artificial Sequence
  Primer Sequence
acgttggatggtttcgtctggatcgcactg

SEQ ID NO: 14 - DNA, 29 bp, Artificial Sequence
  Primer Sequence
acgttggatgtgggtttcgcccagcgcat

SEQ ID NO: 15 - DNA, 17 bp, Artificial Sequence
  Primer Sequence
ttagagacaccaccacg

SEQ ID NO: 16 - DNA, 30 bp, Artificial Sequence
  Primer Sequence
acgttggatggcgctacagtacagcgataa

SEQ ID NO: 17 - DNA, 29 bp, Artificial Sequence
  Primer Sequence
acgttggatgagacggaacgtttcgtctc

SEQ ID NO: 18 - DNA, 15 bp, Artificial Sequence
  Primer Sequence
tcgggcgaacgcggt

SEQ ID NO: 19 - DNA, 30 bp, Artificial Sequence
  Primer Sequence
acgttggatgaatgctgaatggagagccga

SEQ ID NO: 20 - DNA, 30 bp, Artificial Sequence
  Primer Sequence
acgttggatgttgaacagcaactcaaccag

SEQ ID NO: 21 - DNA, 16 bp, Artificial Sequence
  Primer Sequence
cggaagcggggacgga

SEQ ID NO: 22 - DNA, 30 bp, Artificial Sequence
  Primer Sequence
acgttggatggatccgatgctacgagaaag

SEQ ID NO: 23 - DNA, 30 bp, Artificial Sequence
  Primer Sequence
acgttggatggtttgaaccatgtacacggc

SEQ ID NO: 24 - DNA, 15 bp, Artificial Sequence
  Primer Sequence
acacggctggaccat

SEQ ID NO: 25 - DNA, 30 bp, Artificial Sequence
  Primer Sequence
acgttggatgtggcccgtccaaagacaaca

SEQ ID NO: 26 - DNA, 30 bp, Artificial Sequence
  Primer Sequence
acgttggatgatcaggccttcaacgatacc

SEQ ID NO: 27 - DNA, 22 bp, Artificial Sequence
  Primer Sequence
gaggggaaacgatgtcctggcc

SEQ ID NO: 28 - DNA, 996 bp, Klebsiella pneumoniae
  misc_feature (420)..(420): n is t or a
  misc_feature (456)..(456): n is c or t
atgactatcaaagtaggtatcaacggttttggccgtatcggtcgcattgttttccgtgctgctcagaaacgttctgacatcgagatcgttgcaatcaacgacctgttagacgcagagtacatggcttacatgctgaagtatgactccactcacggtcgtttcgacggtaccgttgaagtgaaagacggtcatctggtcgttaacggtaaaaaaatccgtgttaccgctgaacgtgacccggctaacctgaagtgggacgaagttggtgttgacgttgttgctgaagcaaccggtatcttcctgaccgacgaaaccgctcgtaaacacatcaccgctggcgcgaaaaaagtcgttctgactggcccgtccaaagacaacactccgatgttcgttcgcggcgctaacttcgacgcttacgcnggccaggacatcgtttccaacgcttcctgcaccacnaactgcctggcgccgctggctaaagttatcaacgacaacttcggtatcgttgaaggcctgatgaccaccgtccacgctaccaccgctactcagaaaaccgttgatggcccgtctcacaaagactggcgcggcggccgcggcgcagctcagaacatcatcccgtcctctaccggcgctgctaaagcagtaggtaaagtactgccagaactgaacggcaaactgaccggtatggcgttccgcgttccgactccgaacgtatctgttgttgacctgaccgttcgtctggaaaaagcagcgtcctacgaagaaatcaagaaagccatcaaagccgcttctgaaggcgctatgaaaggcgttctgggttacaccgaagacgacgttgtttctaccgacttcaacggcgaagtttgcacttccgtgttcgatgctaaagcgggtatcgcactgaacgacaacttcgtgaaactggtttcctggtacgacaacgaaaccggctactccaacaaagttctggatctgattgcccacatctccaaataa

SEQ ID NO: 29 - DNA, 2691 bp, Klebsiella pneumoniae
  misc_feature (1608)..(1608): n is c or t
atgacagatgtgactattaaagcgctggcctcagagattcagacctctgtggatcgcctgatacagcaatttgctgacgcaggcatccgcaaatcggctgatgattctgtgacctcgcaagagaaacaaactttgttgacgcacctgaaccgtgaacacggctcggcgccagacaagctgacgttacagcgtaagacgcgcagtacgttgaatattccaggtaccggtggaaagagtaaatcggtacaaatcgaagtccgcaagaaacgcacctttgtgaaacgcgatccgcaagaggctgaacgcctggccgcggaagagcaggcgcagcgtgaagcggaagagcaggcccgtcgtgaagctgaagaagcagcgaaacgcgaggcgcaattaaaagctgaacgtgaggccgcagaacaagctaaacgtgaagtcgctgataaagcgaaacgtgaagctgcggaaaaagacaaagtgagcaatcaacataccgacgaaatgaccaaaaccgcccaggctgaaaagatccgtcgcgagaacgaagccgcggaattgaagcgcaaatcggaagaagaagcacgccgcaaacttgaagaagaagcgcgccgtgtagcggaagaagcacgccgtatggctgaagaaaacgaaaaaaattggtctgaaacctcagacagcccggaagatagcagcgactatcacgtcaccacatcacagcatgctcgtcaggctgaagatgataacgatcgtgaagtcgaaggcggtcgcggccgtagccgtagcagcaaagcggctcgtccggcgaagaaaggcaacaaacacgctgaatcgaaagctgatcgtgaagaagcccgcgcggccgttcgcggcggtaaaggcggtaagcaccgtaaaggttccgctctgcagcagggcttccagaagccagcgcaggccgttaaccgtgacgtcgtaatcggcgaaaccatcaccgttggcgaactggctaacaagatggcggtgaaaggttctcaggtcatcaaagcgatgatgaagctgggcgccatggcgaccatcaaccaggtcatcgaccaggaaaccgcacagctggttgccgaagagatgggccacaaagttatcctgcgtcgtgaaaacgaactggaagaagccgtaatgagcgaccgtgacaccggcgcggcggctgaaccgcgcgcaccggtcgtgaccattatgggtcacgttgaccacggtaaaacctcgctgctggactacattcgttctaccaaggttgcctccggcgaagcgggtggtattacccagcacatcggtgcttaccacgtcgaaaccgacaacggcatgatcaccttcctggataccccgggccacgccgcgtttacctccatgcgtgctcgtggcgcgcaggcgacggatatcgtggttctggtggtggcggcagacgacggcgtgatgccgcagactatcgaagctatccagcacgctaaagcggcgcaggtaccggtggtagtggcggtgaacaagatcgataagccagaagccgatccggatcgcgtgaagaacgaactgtcccagtacggcatcctgccggaagagtggggcggcgagagccagttngtccacgtttccgcgaaagcgggtaccggcatcgacgacctgctggacgcgatcctgctgcaggctgaagttcttgagctgaaagcggtccgcaacggtatggcgagcggcgcggtcatcgaatccttccttgataaaggtcgtggtccggtagctaccgttctggttcgcgaaggtactctgcacaagggcgacattgttctgtgcggcttcgaatatggccgtgtgcgcgcgatgcgtgacgaactgggtcgcgaagtgctggaagcgggtccgtccattccggtggaaatcctcggcctgtccggtgtgccggctgccggtgatgaagtgaccgtagtgcgtgacgagaaaaaagcgcgtgaagtggcgctgtatcgtcagggcaaattccgtgaagttaagctggcgcgtcagcagaaatctaaactggaaaacatgttcgctaacatgaccgaaggcgaagttcacgaagtgaacatcgtactgaaagcggacgtacagggttctgtcgaagcgatttccgattccttactgaaactgtctaccgacgaagtgaaagtgaagatcatcggttccggcgtaggtggtatcaccgaaaccgacgctaccctggcagcagcatccaacgcgattctggttggcttcaacgttcgtgccgatgcctctgcgcgtaaagttatcgaagcggaaagcctggatctgcgttactactccgtcatctataacctgatcgacgaagtgaaagcggcgatgagcggcatgctgtctccggaactgaaacagcagatcatcggtctggctgaagtgcgtgatgtcttcaaatcgccgaaattcggcgccatcgcgggctgtatggttaccgaagggacgatcaaacgtcacaacccaatccgcgttctgcgtgacaacgtggttatctatgaaggcgagctggaatccctgcgccgcttcaaagatgacgttaacgaagtccgtaacggcatggaatgtggtatcggtgtgaagaactacaacgacgttcgcgttggcgatatgatcgaagtgttcgaaatcatcgaaatccagcgtagcatcgattaa

SEQ ID NO: 30 - DNA, 16 bp, Artificial Sequence
  Primer Sequence
  misc_feature (5)..(5): n is inosine
  modified_base (5)..(5): I
ccgcntcctgcaccac

SEQ ID NO: 31 - DNA, 30 bp, Artificial Sequence
  Primer Sequence
acgttggatgtgaagaacgaactgtcccag

SEQ ID NO: 32 - DNA, 29 bp, Artificial Sequence
  Primer Sequence
acgttggatggtccagcaggtcgtcgatg

SEQ ID NO: 33 - DNA, 20 bp, Artificial Sequence
  Primer Sequence
gtgtgcggcgagagccagtt

SEQ ID NO: 34 - DNA, 30 bp, Artificial Sequence
  Primer Sequence
acgttggatggggtcaccattctgccttta

SEQ ID NO: 35 - DNA, 29 bp, Artificial Sequence
  Primer Sequence
acgttggatgcgcccgctttcgcttccac

SEQ ID NO: 36 - DNA, 21 bp, Artificial Sequence
  Primer Sequence
  misc_feature (10)..(10): n is inosine
  modified_base (10)..(10): I
  misc_feature (13)..(13): n is inosine
  modified_base (13)..(13): I
aagtattcanaangccggtac

SEQ ID NO: 37 - DNA, 30 bp, Artificial Sequence
  Primer Sequence
acgttggatgacaatatctacctggcgacc

SEQ ID NO: 38 - DNA, 30 bp, Artificial Sequence
  Primer Sequence
acgttggatggttgaagtagtaggtcaggc

SEQ ID NO: 39 - DNA, 16 bp, Artificial Sequence
  Primer Sequence
ggctgatcggggtcat

SEQ ID NO: 40 - DNA, 29 bp, Artificial Sequence
  Primer Sequence
acgttggatgtccctcggctatgtgctgt

SEQ ID NO: 41 - DNA, 30 bp, Artificial Sequence
  Primer Sequence
acgttggatgcgaaggcgttcatgtttttg

SEQ ID NO: 42 - DNA, 17 bp, Artificial Sequence
  Primer Sequence
  misc_feature (6)..(6): n is inosine
  modified_base (6)..(6): I
gatatngaaggggtggg

SEQ ID NO: 43 - DNA, 30 bp, Artificial Sequence
  Primer Sequence
acgttggatgtgattaactccctgtccgtg

SEQ ID NO: 44 - DNA, 30 bp, Artificial Sequence
  Primer Sequence
acgttggatgccttcttcgatagcagacag

SEQ ID NO: 45 - DNA, 22 bp, Artificial Sequence
  Primer Sequence
  misc_feature (4)..(4): n is inosine
  modified_base (4)..(4): I
ggcnccgtatcgtaaagtgacc

SEQ ID NO: 46 - DNA, 20 bp, Artificial Sequence
  Primer Sequence
ctattgtgttggccgtctac

SEQ ID NO: 47 - DNA, 20 bp, Artificial Sequence
  Primer Sequence
aatccctcgagcgcgagtct

SEQ ID NO: 48 - DNA, 20 bp, Artificial Sequence
  Primer Sequence
ccaatattatgcacccggtc

SEQ ID NO: 49 - DNA, 20 bp, Artificial Sequence
  Primer Sequence
gagctggcggaaaaccagat

SEQ ID NO: 50 - DNA, 15 bp, Artificial Sequence
  Primer Sequence
ctgagcgggtgcatg

SEQ ID NO: 51 - DNA, 20 bp, Artificial Sequence
  Primer Sequence
atgttggtgttcgtttccgc

SEQ ID NO: 52 - DNA, 20 bp, Artificial Sequence
  Primer Sequence
gtttaaaagacgagcacgga

SEQ ID NO: 53 - DNA, 15 bp, Artificial Sequence
  Primer Sequence
gtaatggcgtctgcg

SEQ ID NO: 54 - DNA, 19 bp, Artificial Sequence
  Primer Sequence
gagaataagcagcaaggat

SEQ ID NO: 55 - DNA, 20 bp, Artificial Sequence
  Primer Sequence
ccatcccacttaaagacttg

SEQ ID NO: 56 - DNA, 20 bp, Artificial Sequence
  Primer Sequence
gcgggtaaaaatgcttggtt

SEQ ID NO: 57 - DNA, 20 bp, Artificial Sequence
  Primer Sequence
gtttcgtctggatcgcactg

SEQ ID NO: 58 - DNA, 19 bp, Artificial Sequence
  Primer Sequence
tgggtttcgcccagcgcat

SEQ ID NO: 59 - DNA, 15 bp, Artificial Sequence
  Primer Sequence
agagacaccaccacg

SEQ ID NO: 60 - DNA, 20 bp, Artificial Sequence
  Primer Sequence
gcgctacagtacagcgataa

SEQ ID NO: 61 - DNA, 19 bp, Artificial Sequence
  Primer Sequence
agacggaacgtttcgtctc

SEQ ID NO: 62 - DNA, 20 bp, Artificial Sequence
  Primer Sequence
aatgctgaatggagagccga

SEQ ID NO: 63 - DNA, 20 bp, Artificial Sequence
  Primer Sequence
ttgaacagcaactcaaccag

SEQ ID NO: 64 - DNA, 15 bp, Artificial Sequence
  Primer Sequence
ggaagcggggacgga

SEQ ID NO: 65 - DNA, 20 bp, Artificial Sequence
  Primer Sequence
gatccgatgctacgagaaag

SEQ ID NO: 66 - DNA, 20 bp, Artificial Sequence
  Primer Sequence
gtttgaaccatgtacacggc

SEQ ID NO: 67 - DNA, 20 bp, Artificial Sequence
  Primer Sequence
tggcccgtccaaagacaaca

SEQ ID NO: 68 - DNA, 20 bp, Artificial Sequence
  Primer Sequence
atcaggccttcaacgatacc

SEQ ID NO: 69 - DNA, 18 bp, Artificial Sequence
  Primer Sequence
ggaaacgatgtcctggcc

SEQ ID NO: 70 - DNA, 939 bp, Klebsiella pneumoniae
  misc_feature (633)..(633): n is t or c
atgaaagttgcagtccttggcgctgccggtggtatcggccaggcgcttgccctactacttaagacccaactgccttcaggttcagagctctcgttgtacgacatcgctccggttacgccgggcgtggcggtagatctaagtcatatccccacagatgtaaaaattaaaggattttccggtgaagacgctactccggcgctggaaggcgcggatgtagtgctgatctccgcgggcgtggcgcgtaagcccggcatggatcgttccgacctgtttaatgtgaatgcgggtatcgtgaagaacctcgtgcagcagattgccaaaacctgcccgcaggcctgcatcggcattatcaccaacccggtgaataccaccgtggctatcgccgccgaagtactgaaaaaagccggcgtgtacgataaaaacaaactgttcggcgttaccacgctggacatcatccgttccaatacctttgtggcggagctgaaaggtaaatcggcaaccgaggtggaagtcccggtcattggtggtcactccggggtcaccattctgcctttactgtcgcagatccccggcgtcagctttagcgatcaggaaattgccgacctgactaaacgtattcagaacgccggtacngaagtcgtggaagcgaaagcgggcggcgggtcggcgaccttgtcgatgggccaggcggctgcccgttttggtctctctctggttcgcgccatgcagggggaaaaaggcgtggtggagtgcgcctacgtggaaggcgacggccactatgcgcgtttcttctcccagccgctgctgctggggaaaaacggcgtggaagagcgacagtctatcggcaaactcagcgcctttgaacagcaggcgctggagggcatgctggacaccctgaagaaagatatcgcgctcggcgaagacttcgtcaacaagtaa

SEQ ID NO: 71 - DNA, 1029 bp, Klebsiella pneumoniae
  misc_feature (732)..(732): n is g or a
  misc_feature (867)..(867): n is g or a
atgatgatgggctttgtggcttcaacagcgacgcaggcagcggaagtttataataagaacgcgaacaagctggatgtgtacggcaagatcaaagccatgcactatttcagcgactatgacagcaaggatggcgatcagacctacgtgcgtttcggtattaaaggcgaaacgcagattaacgacgacctgaccggctatggccgttgggaatctgaattctccggtaacaaaaccgagagcgactccagccagaaaacccgtctggcgttcgccggcgtgaagctgaagaactacggctccttcgattacggtcgtaaccttggcgccctgtacgacgtggaagcctggaccgatatgttcccggaattcggcggcgattcctctgcccagaccgataactttatgaccaagcgcgccagcggcctggcgacctaccgcaacaccgacttcttcggtctggtggatggcctggatctgaccctgcagtaccagggtaaaaacgaaggccgtgaagcgaagaaacagaacggcgacggcgtcggcacctcgttaagctatgatttcggcggcagcgacttcgccgtcagcgcagcctacaccagctccgaccgtaccaacgatcagaacctgctggcccgcggccagggttcgaaagcggaagcctgggcgaccggcctgaaatatgacgccaacaatatctacctggcgaccatgtactctgaaacccgcaanatgaccccgatcagcggcggctttgccaacaaagcgcagaactttgaagcggtggcgcagtatcagttcgacttcggtctgcgtccgtccctcggctatgtgctgtcgaaagggaaggatatcgaaggggtgggnagtgaagatctggttaactacatcgacgtgggcctgacctactacttcaacaaaaacatgaacgccttcgtggattacaaaatcaaccagctgaaaagcgataacaaactcggcatcaacgatgacgacatcgtcgcgctgggtatgacctaccagttctga

SEQ ID NO: 72 - DNA, 15 bp, Artificial Sequence
  Primer Sequence
  misc_feature (4)..(4): n is inosine
  modified_base (4)..(4): I
cgcntcctgcaccac

SEQ ID NO: 73 - DNA, 20 bp, Artificial Sequence
  Primer Sequence
tgaagaacgaactgtcccag

SEQ ID NO: 74 - DNA, 19 bp, Artificial Sequence
  Primer Sequence
gtccagcaggtcgtcgatg

SEQ ID NO: 75 - DNA, 16 bp, Artificial Sequence
  Primer Sequence
gcggcgagagccagtt

SEQ ID NO: 76 - DNA, 20 bp, Artificial Sequence
  Primer Sequence
gggtcaccattctgccttta

SEQ ID NO: 77 - DNA, 19 bp, Artificial Sequence
  Primer Sequence
cgcccgctttcgcttccac

SEQ ID NO: 78 - DNA, 17 bp, Artificial Sequence
  Primer Sequence
  misc_feature (6)..(6): n is inosine
  modified_base (6)..(6): I
  misc_feature (9)..(9): n is inosine
  modified_base (9)..(9): I
attcanaangccggtac

SEQ ID NO: 79 - DNA, 20 bp, Artificial Sequence
  Primer Sequence
acaatatctacctggcgacc

SEQ ID NO: 80 - DNA, 20 bp, Artificial Sequence
  Primer Sequence
gttgaagtagtaggtcaggc

SEQ ID NO: 81 - DNA, 15 bp, Artificial Sequence
  Primer Sequence
gctgatcggggtcat

SEQ ID NO: 82 - DNA, 19 bp, Artificial Sequence
  Primer Sequence
tccctcggctatgtgctgt

SEQ ID NO: 83 - DNA, 20 bp, Artificial Sequence
  Primer Sequence
cgaaggcgttcatgtttttg

SEQ ID NO: 84 - DNA, 20 bp, Artificial Sequence
  Primer Sequence
tgattaactccctgtccgtg

SEQ ID NO: 85 - DNA, 20 bp, Artificial Sequence
  Primer Sequence
ccttcttcgatagcagacag

SEQ ID NO: 86 - DNA, 20 bp, Artificial Sequence
  Primer Sequence
  misc_feature (2)..(2): n is inosine
  modified_base (2)..(2): I
cnccgtatcgtaaagtgacc

SEQ ID NO: 87 - DNA, 882 bp, Klebsiella pneumoniae
  misc_feature (814)..(814): n is c or t
atgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagnacagcgaggccgtcatcgccgctgcggctagactcgcgctcgagggattgggcgtcaacgggcagtaa

SEQ ID NO: 88 - DNA, 813 bp, Klebsiella pneumoniae
  misc_feature (82)..(82): n is c or g
atggaattgcccaatattatgcacccggtcgcgaagctgagcaccgcattagccgctgcattgatgctgagcgggtgcatgnccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 89 - DNA, 825 bp, Klebsiella pneumoniae
atgaaaaaaattttgctgctgcatatgttggtgttcgtttccgccactctcccaatcagttccgtggcttctgatgaggttgaaacgcttaaatgcaccatcatcgcagacgccattaccggaaataccttatatgagaccggagaatgtgcccgtcgtgtgtctccgtgctcgtcttttaaacttccattggcaatcatggggtttgatagtggaatcttgcagtcgccaaaatcacctacgtgggaattgaagccggaatacaacccgtctccgagagatcgcacatacaaacaagtctatccggcgctatggcaaagcgactctgttgtctggttctcgcagcaattaacaagccgtctgggagttgatcggttcacggaatacgtaaagaaatttgagtacggtaatcaagatgtttccggtgactcggggaagcataacggcttgacccagtcatggctgatgtcgtcgctcaccatatctcccaaggagcaaattcagtttcttctacgctttgtcgcgcataagctgcctgtatccgaagcggcttatgacatggcgtatgccacaatcccgcagtaccaggcagccgaaggatgggctgtacatggaaaaagcggcagcggctggcttcgggacaataacggcaagataaatgaaagtcgtccgcagggctggttcgtgggctgggctgaaaaaaacggacggcaagttgttttcgcccgattggaaataggaaaggaaaagtccgatattcccggcgggtctaaagcacgagaggatattctcgtggaattacccgtgttgatgggtaacaaatga

SEQ ID NO: 90 - DNA, 798 bp, Klebsiella pneumoniae
  misc_feature (186)..(186): n is g or a
atgcgtgtattagccttatcggctgtgtttttggtggcatcgattatcggaatgcctgcggtagcaaaggaatggcaagaaaacaaaagttggaatgctcactttactgaacataaatcacagggcgtagttgtgctctggaatgagaataagcagcaaggatttaccaataatcttaaacgggcnaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 91 - DNA, 876 bp, Escherichia coli
  misc_feature (556)..(556): n is c or g
atggtgacaaagagagtgcaacggatgatgttcgcggcggcggcgtgcattccgctgctgctgggcagcgcgccgctttatgcgcagacgagtgcggtgcagcaaaagctggcggcgctggagaaaagcagcggagggcggctgggcgtcgcgctcatcgataccgcagataatacgcaggtgctttatcgcggtgatgaacgctttccaatgtgcagtaccagtaaagttatggcggccgcggcggtgcttaagcagagtgaaacgcaaaagcagctgcttaatcagcctgtcgagatcaagcctgccgatctggttaactacaatccgattgccgaaaaacacgtcaacggcacaatgacgctggcagagctgagcgcggccgcgttgcagtacagcgacaataccgccatgaacaaattgattgcccagctcggtggcccgggaggcgtgacggcttttgcccgcgcgatcggcgatgagacgtttcgtctggatcgcactgaacctacgctgaataccgccattcccggcgacccgagagacaccaccacgncgcgggcgatggcacagacgttgcgtcagcttacgctgggtcatgcgctgggcgaaacccagcgggcgcagttggtgacgtggctcaaaggcaatacgaccggcgcagccagcattcgggccggcttaccgacgtcgtggactgcaggtgataagaccggcagcggcgactacggcaccaccaatgatattgcggtgatctggccgcagggtcgtgcgccgctggttctggtgacctattttacccagccgcaacagaacgcagagagccgccgcgatgtgctggcttcagcggcgagaatcatcgccgaagggctgtaa

SEQ ID NO: 92 - DNA, 876 bp, Escherichia coli
  misc_feature (453)..(453): n is c or t
atggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtnaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 93 - DNA, 519 bp, Klebsiella pneumoniae
  misc_feature (223)..(223): n is a or c or t
  misc_feature (454)..(454): n is t or g
atgactgagcatgaccttgcgatgctctatgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacgganggtgggaagaagaaaccgatccaggagtacgcggaatagaccagttactggcgaatgcatcacaactgggcaaaggcttgggaaccaagctggttcgagctctggttgagttgctgttcaatgatcccgaggtcaccaagatccaaacggacccgtcgccgagcaacttgcgagcgatccgatgctacgagaaagcggggtttgagaggcaaggtaccgtaaccaccccanatggtccagccgtgtacatggttcaaacacgccaggcattcgagcgaacacgcagtgatgcctaa

SEQ ID NO: 94 - DNA, 4062 bp, Klebsiella pneumoniae
  misc_feature (1819)..(1819): n is a or g
atgggtcgacttgtcagcgagctgaggaaccctatggtttactcctataccgagaaaaaacgtattcgtaaggattttggtaaacgtccacaagttctggatgtgccatatctcctttctatccagcttgactcgttccagaagtttatcgagcaagatcctgaagggcagtatggtctggaagctgctttccgttccgtattcccgattcagagctacagcggtaattccgagctgcaatacgtcagctaccgtctgggcgaaccggtttttgacgttaaagaatgtcagatccgtggcgtgacttattctgcaccgctgcgcgtaaaactgcgtctggtgatctacgagcgcgaagcgccagaaggcaccgtcaaagacattaaagaacaagaagtctacatgggcgaaattccgctcatgaccgacaacggtacctttgttatcaacggtaccgagcgtgttatcgtttctcagctgcaccgtagcccgggcgtcttctttgacagtgacaaaggtaagacccactcctccggtaaggttctgtataacgcacgtatcatcccttaccgcggttcctggctggacttcgaattcgacccgaaagacaacctgttcgttcgtatcgaccgtcgtcgtaagctgccggctaccatcatcctgcgcgcgctgaattacaccactgagcagatccttgacctgttcttcgaaaaagtggtctttgaaattcgcgacaacaagctgcagatggagctgattccggagcgtctgcgcggtgaaactgcctccttcgatatcgaagccaacggcaaagtgtacgttgagaaaggccgtcgcatcacggctcgtcatatccgtcagctggaaaaagacgatatcaagcatatcgaagtgccggttgaatatatcgcgggcaaagtggcggctaaagactatatcgatgaagcgactggcgaactgatctgcccggcgaacatggagctgagcctggatctgctggctaagctgagccagtctggccacaagcgtatcgaaacgctgttcaccaacgatctggaccacggcccgtacatctctgaaacggtacgcgtcgacccgactaacgatcgtctgagcgcgctggtagaaatctaccgcatgatgcgccctggcgagccgccgactcgtgaagccgctgaaagtctgtttgagaacctgttcttctctgaagaccgctacgatctgtccgcggttggtcgtatgaagttcaaccgttctctgctgcgcgacgaaatcgaaggttccggtatcctgagcaaagacgacatcatcgaagtgatgaagaagctcatcgatatccgtaacggtaaaggcgaagtggacgatatcgaccacctcggcaaccgtcgtatccgttccgttggcgaaatggcggaaaaccagttccgcgttggcctggtacgtgtagagcgtgcggtgaaagagcgtctgtctcttggcgatctggataccctgatgcctcaggatatgatcaacgccaagccgatttccgcagcagtgaaagagttctttggttccagccagctgtctcagtttatggaccagaacaacccgctgtctgagattacgcacaaacgtcgtatctccgcactcggcccaggcggtctgacccgtgagcgcgcaggcttcgaagttcgagacgtacacccgacccactacggtcgcgtatgtccgatcgaaacgcctgaaggtccaaacatcggtctgattaactccctgtccgtgtacgcgcagaccaacgaatatggcttccttgagacgccgtatcgtaaagtgaccnacggtgtggttactgacgaaattcactacctgtctgctatcgaagaaggcaactacgttatcgctcaggcgaactccaacctggatgaaaacggccacttcgtagaagatctggttacctgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggcggttccgactctgcgcgctgataagccgctggttggtaccggtatggaacgtgctgttgccgttgactccggtgttactgccgtggctaaacgtggcggtaccgttcagtacgtggatgcttcccgtatcgttatcaaagttaacgaagacgagatgtacccgggcgaagcaggtatcgacatctataacctgaccaagtacacccgttctaaccagaacacctgcatcaaccagatgccttgcgtgtccctgggcgaacctattgagcgcggcgacgtgctggcagacggcccgtccaccgacctcggtgagctggcgctgggtcagaacatgcgtgtagcgttcatgccgtggaacggttacaacttcgaagactccatcctcgtttccgagcgtgttgtccaggaagaccgtttcaccactatccacattcaggaactggcgtgcgtgtcccgtgacaccaagctggggccggaagagatcaccgctgacatcccgaacgtgggtgaagctgcgctctccaaactggatgaatccggtatcgtttatatcggtgcggaagtgaccggcggcgacattctggttggtaaggtaacgccgaaaggtgaaacccagctgacgccggaagagaaactgctgcgcgcgatcttcggtgagaaagcgtctgatgttaaagactcttctctgcgcgtaccgaacggtgtctctggtaccgttatcgacgttcaggtctttacccgtgacggcgtagaaaaagacaaacgtgctctggaaatcgaagagatgcagctgaagcaggccaaaaaagacctgtctgaagaactgcagatcctcgaagccggtctgttcagtcgtatctacgcggtgctggtctccggtggcgttgaagctgagaagctcgacaagctgcctcgcgatcgctggctggaactgggcctgaccgacgaagagaaacaaaatcagctggaacagctggctgagcagtatgacgaactgaaacacgagttcgagaaaaaactcgaagcgaaacgccgtaaaatcacccaaggcgacgatctggcaccgggcgtgctgaagattgttaaggtatatctggccgttaaacgccgtatccagcctggtgataagatggcaggtcgtcacggtaacaagggtgttatctctaagatcaacccgatcgaagatatgccgcacgatgctaacggtacgccggtagatatcgtactgaacccgctgggcgtaccgtctcgtatgaacatcggtcagatccttgaaactcacctgggtatggctgcaaaaggcatcggcgataagatcaacgccatgctgaagcagcagcaggaagtcgcgaaactgcgcgaattcatccagcgtgcttacgatctgggcgctgacgttcgccagaaagttgacctgaataccttcagcgatgaagaagttctgcgtctggctgaaaacctgcgtaaaggcatgccgatcgcaacgccggtattcgacggtgcgaaagaagctgaaatcaaagagctgctgcagctgggcgacctgccgacttccggtcagatcaccctgtttgatggccgcaccggtgaacagttcgaacgtccggtaaccgtgggttacatgtacatgctgaaactgaaccacctggtcgacgacaaaatgcacgctcgttccaccggttcttacagcctggttactcagcagccgctgggtggtaaggcacagttcggtggtcagcgcttcggtgagatggaagtgtgggcgctggaagcatacggcgcggcctacaccctgcaggaaatgctcaccgttaagtctgatgacgtgaacggtcgtaccaagatgtataaaaacatcgtggacggcaatcaccagatggaaccgggcatgccggaatccttcaacgtactgttgaaagaaattcgctcgctgggcatcaacatcgaactggaagacgagtaa

SEQ ID NO: 95 - DNA, 30 bp, Artificial Sequence
  Primer Sequence
acgttggatgtgttgtttgggtttcgcaag

SEQ ID NO: 96 - DNA, 30 bp, Artificial Sequence
  Primer Sequence
acgttggatgccagagaagtcttgatttcc

SEQ ID NO: 97 - DNA, 27 bp, Artificial Sequence
  Primer Sequence
ccaaaaaattggattaaataaaatcaa

SEQ ID NO: 98 - DNA, 30 bp, Artificial Sequence
  Primer Sequence
acgttggatggcatttctcaggaagctcta

SEQ ID NO: 99 - DNA, 29 bp, Artificial Sequence
  Primer Sequence
acgttggatgccttcccagcccgtctttg

SEQ ID NO: 100 - DNA, 17 bp, Artificial Sequence
  Primer Sequence
cagcgcttggtcaagga

SEQ ID NO: 101 - DNA, 30 bp, Artificial Sequence
  Primer Sequence
acgttggatggatccttgagagttttcgcc

SEQ ID NO: 102 - DNA, 30 bp, Artificial Sequence
  Primer Sequence
acgttggatggcttttctgtgactggtgag

SEQ ID NO: 103 - DNA, 20 bp, Artificial Sequence
  Primer Sequence
gttaaagttctgctatgtgg

SEQ ID NO: 104 - DNA, 30 bp, Artificial Sequence
  Primer Sequence
acgttggatgaggtagcgttgccaatgatg

SEQ ID NO: 105 - DNA, 30 bp, Artificial Sequence
  Primer Sequence
acgttggatgtgcttgatagtcggaagagg

SEQ ID NO: 106 - DNA, 21 bp, Artificial Sequence
  Primer Sequence
ataatcggaagaggcataaat

SEQ ID NO: 107 - DNA, 30 bp, Artificial Sequence
  Primer Sequence
acgttggatgatacgcggaaggcaataacg

SEQ ID NO: 108 - DNA, 30 bp, Artificial Sequence
  Primer Sequence
acgttggatgccaatcgatttaagtgaggc

SEQ ID NO: 109 - DNA, 15 bp, Artificial Sequence
  Primer Sequence
ggcatgcaccatcaa

SEQ ID NO: 110 - DNA, 30 bp, Artificial Sequence
  Primer Sequence
acgttggatgtcaccgtcagatctacgaac

SEQ ID NO: 111 - DNA, 30 bp, Artificial Sequence
  Primer Sequence
acgttggatgcatggtgccggttttttcag

SEQ ID NO: 112 - DNA, 21 bp, Artificial Sequence
  Primer Sequence
cccccacggtgtaccgcaggc

SEQ ID NO: 113 - DNA, 30 bp, Artificial Sequence
  Primer Sequence
acgttggatgttctggaattcgacgtaccg

SEQ ID NO: 114 - DNA, 30 bp, Artificial Sequence
  Primer Sequence
acgttggatgtgataaacacgaccagacgg

SEQ ID NO: 115 - DNA, 17 bp, Artificial Sequence
  Primer Sequence
gtaccggacgaactgat

SEQ ID NO: 116 - DNA, 30 bp, Artificial Sequence
  Primer Sequence
acgttggatgattcaacccgccgaaagtag

SEQ ID NO: 117 - DNA, 30 bp, Artificial Sequence
  Primer Sequence
acgttggatgcttcctgatcatctttacgg

SEQ ID NO: 118 - DNA, 25 bp, Artificial Sequence
  Primer Sequence
gcaaagacgacgttaccggtgaaga

SEQ ID NO: 119 - DNA, 30 bp, Artificial Sequence
  Primer Sequence
acgttggatgcggtgaaaaatccacacagg

SEQ ID NO: 120 - DNA, 30 bp, Artificial Sequence
  Primer Sequence
acgttggatgatggcaacgcgatattcacg

SEQ ID NO: 121 - DNA, 17 bp, Artificial Sequence
  Primer Sequence
caggtttatggtcagga

SEQ ID NO: 122 - DNA, 30 bp, Artificial Sequence
  Primer Sequence
acgttggatggaaaattcgcttcccggaac

SEQ ID NO: 123 - DNA, 29 bp, Artificial Sequence
  Primer Sequence
acgttggatggaaccagacgtttggtgcc

SEQ ID NO: 124 - DNA, 23 bp, Artificial Sequence
  Primer Sequence
ggtatcggtattaagccgtgttc

SEQ ID NO: 125 - DNA, 30 bp, Artificial Sequence
  Primer Sequence
acgttggatgtatcgggcctgcttatgaag

SEQ ID NO: 126 - DNA, 30 bp, Artificial Sequence
  Primer Sequence
acgttggatgttgtcgaaaaggtcgccaac

SEQ ID NO: 127 - DNA, 21 bp, Artificial Sequence
  Primer Sequence
gcctgcttatgaagataaagt

SEQ ID NO: 128 - DNA, 30 bp, Artificial Sequence
  Primer Sequence
acgttggatgagccgaatgctgatttcctg

SEQ ID NO: 129 - DNA, 30 bp, Artificial Sequence
  Primer Sequence
acgttggatgcctgagttttatgcactgcc

SEQ ID NO: 130 - DNA, 19 bp, Artificial Sequence
  Primer Sequence
atatcggcattatggatgg

SEQ ID NO: 131 - DNA, 29 bp, Artificial Sequence
  Primer Sequence
acgttggatggcctgatgaaagcagtgcg

SEQ ID NO: 132 - DNA, 30 bp, Artificial Sequence
  Primer Sequence
acgttggatgattcgtggatctctgctttg

SEQ ID NO: 133 - DNA, 19 bp, Artificial Sequence
  Primer Sequence
tgaaagcagtgcgccgttt

SEQ ID NO: 134 - DNA, 20 bp, Artificial Sequence
  Primer Sequence
tgttgtttgggtttcgcaag

SEQ ID NO: 135 - DNA, 20 bp, Artificial Sequence
  Primer Sequence
ccagagaagtcttgatttcc

SEQ ID NO: 136 - DNA, 20 bp, Artificial Sequence
  Primer Sequence
gcatttctcaggaagctcta

SEQ ID NO: 137 - DNA, 19 bp, Artificial Sequence
  Primer Sequence
ccttcccagcccgtctttg

SEQ ID NO: 138 - DNA, 20 bp, Artificial Sequence
  Primer Sequence
gatccttgagagttttcgcc

SEQ ID NO: 139 - DNA, 20 bp, Artificial Sequence
  Primer Sequence
gcttttctgtgactggtgag

SEQ ID NO: 140 - DNA, 19 bp, Artificial Sequence
  Primer Sequence
ttaaagttctgctatgtgg

SEQ ID NO: 141 - DNA, 20 bp, Artificial Sequence
  Primer Sequence
aggtagcgttgccaatgatg

SEQ ID NO: 142 - DNA, 20 bp, Artificial Sequence
  Primer Sequence
tgcttgatagtcggaagagg

SEQ ID NO: 143 - DNA, 17 bp, Artificial Sequence
  Primer Sequence
tcggaagaggcataaat

SEQ ID NO: 144 - DNA, 20 bp, Artificial Sequence
  Primer Sequence
atacgcggaaggcaataacg

SEQ ID NO: 145 - DNA, 20 bp, Artificial Sequence
  Primer Sequence
ccaatcgatttaagtgaggc

SEQ ID NO: 146 - DNA, 20 bp, Artificial Sequence
  Primer Sequence
tcaccgtcagatctacgaac

SEQ ID NO: 147 - DNA, 20 bp, Artificial Sequence
  Primer Sequence
catggtgccggttttttcag

SEQ ID NO: 148 - DNA, 17 bp, Artificial Sequence
  Primer Sequence
cacggtgtaccgcaggc

SEQ ID NO: 149 - DNA, 20 bp, Artificial Sequence
  Primer Sequence
ttctggaattcgacgtaccg

SEQ ID NO: 150 - DNA, 20 bp, Artificial Sequence
  Primer Sequence
tgataaacacgaccagacgg

SEQ ID NO: 151 - DNA, 20 bp, Artificial Sequence
  Primer Sequence
attcaacccgccgaaagtag

SEQ ID NO: 152 - DNA, 20 bp, Artificial Sequence
  Primer Sequence
cttcctgatcatctttacgg

SEQ ID NO: 153 - DNA, 20 bp, Artificial Sequence
  Primer Sequence
cggtgaaaaatccacacagg

SEQ ID NO: 154 - DNA, 20 bp, Artificial Sequence
  Primer Sequence
atggcaacgcgatattcacg

SEQ ID NO: 155 - DNA, 20 bp, Artificial Sequence
  Primer Sequence
gaaaattcgcttcccggaac

SEQ ID NO: 156 - DNA, 19 bp, Artificial Sequence
  Primer Sequence
gaaccagacgtttggtgcc

SEQ ID NO: 157 - DNA, 20 bp, Artificial Sequence
  Primer Sequence
tatcgggcctgcttatgaag

SEQ ID NO: 158 - DNA, 20 bp, Artificial Sequence
  Primer Sequence
ttgtcgaaaaggtcgccaac

SEQ ID NO: 159 - DNA, 20 bp, Artificial Sequence
  Primer Sequence
agccgaatgctgatttcctg

SEQ ID NO: 160 - DNA, 20 bp, Artificial Sequence
  Primer Sequence
cctgagttttatgcactgcc

SEQ ID NO: 161 - DNA, 19 bp, Artificial Sequence
  Primer Sequence
gcctgatgaaagcagtgcg

SEQ ID NO: 162 - DNA, 20 bp, Artificial Sequence
  Primer Sequence
attcgtggatctctgctttg

SEQ ID NO: 163 - DNA, 831 bp, Escherichia coli
  misc_feature (408)..(408): n is g or a
atgaaaaacacaatacatatcaacttcgctatttttttaataattgcaaatattatctacagcagcgccagtgcatcaacagatatctctactgttgcatctccattatttgaaggaactgaaggttgttttttactttacgatgcatccacaaacgctgaaattgctcaattcaataaagcaaagtgtgcaacgcaaatggcaccagattcaactttcaagatcgcattatcacttatggcatttgatgcggaaataatagatcagaaaaccatattcaaatgggataaaacccccaaaggaatggagatctggaacagcaatcatacaccaaagacgtggatgcaattttctgttgtttgggtttcgcaagaaataacccaaaaaattggattaaataaaatcaanaattatctcaaagattttgattatggaaatcaagacttctctggagataaagaaagaaacaacggattaacagaagcatggctcgaaagtagcttaaaaatttcaccagaagaacaaattcaattcctgcgtaaaattattaatcacaatctcccagttaaaaactcagccatagaaaacaccatagagaacatgtatctacaagatctggataatagtacaaaactgtatgggaaaactggtgcaggattcacagcaaatagaaccttacaaaacggatggtttgaagggtttattataagcaaatcaggacataaatatgtttttgtgtccgcacttacaggaaacttggggtcgaatttaacatcaagcataaaagccaagaaaaatgcgatcaccattctaaacacactaaatttataa

SEQ ID NO: 164 - DNA, 828 bp, Escherichia coli
  misc_feature (585)..(585): n is t or c
atggcaatccgaatcttcgcgatacttttctccattttttctcttgccactttcgcgcatgcgcaagaaggcacgctagaacgttctgactggaggaagtttttcagcgaatttcaagccaaaggcacgatagttgtggcagacgaacgccaagcggatcgtgccatgttggtttttgatcctgtgcgatcgaagaaacgctactcgcctgcatcgacattcaagatacctcatacactttttgcacttgatgcaggcgctgttcgtgatgagttccagatttttcgatgggacggcgttaacaggggctttgcaggccacaatcaagaccaagatttgcgatcagcaatgcggaattctactgtttgggtgtatgagctatttgcaaaggaaattggtgatgacaaagctcggcgctatttgaagaaaatcgactatggcaacgccgatccttcgacaagtaatggcgattactggatagaaggcagccttgcaatctcggcgcaggagcaaattgcatttctcaggaagctctatcgtaacgagctgccctttcgggtagaacatcagcgcttggtcaagganctcatgattgtggaagccggtcgcaactggatactgcgtgcaaagacgggctgggaaggccgtatgggttggtgggtaggatgggttgagtggccgactggctccgtattcttcgcactgaatattgatacgccaaacagaatggatgatcttttcaagagggaggcaatcgtgcgggcaatccttcgctctattgaagcgttaccgcccaacccggcagtcaactcggacgctgcgcgataa

SEQ ID NO: 165 - DNA, 861 bp, Escherichia coli
  misc_feature (228)..(228): n is t or c
atgagtattcaacattttcgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggngcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 166 - DNA, 816 bp, Escherichia coli
  misc_feature (230)..(230): n is a or c
atgagccatattcaacgggaaacgtcttgctcgaggccgcgattaaattccaacctggatgctgatttatatgggtatagatgggctcgcgataatgtcgggcaatcaggtgcgacaatctatcgattgtatgggaagcccaatgcgccagagttgtttctgaaacatggcaaaggtagcgttgccaatgatgttacagatgagatggtcagactaaactggctgacggnatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgctagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctga

SEQ ID NO: 167 - DNA, 861 bp, Escherichia coli
  misc_feature (63)..(63): n is g or c or a
atgcatacgcggaaggcaataacggaggcaattcgaaaactcggagtccaaaccggtgacctnttgatggtgcatgcctcacttaaagcgattggtccggtcgaaggaggagcggagacggtcgttgccgcgttacgctccgcggttgggccgactggcactgtgatgggatacgcgtcgtgggaccgatcaccctacgaggagactctgaatggcgctcggttggatgacaaagcccgccgtacctggccgccgttcgatcccgcaacggccgggacttaccgtgggttcggcctgctgaatcaatttctggttcaagcccccggcgcgcggcgcagcgcgcaccccgatgcatcgatggtcgcggttggtccgctagctgaaacgctgacggagcctcacgaactcggtcacgccttgggggaagggtcgcccgtcgagcggttcgtccgccttggcgggaaggccctgctgttgggtgcgccgctaaactccgttaccgcattgcactacgccgaggcggttgcggatatccccaacaaacgatgggtgacgtatgagatgccgatgcttggaagaaacggtgaagtcgcctggaaaacggcatcagaatacgattcaaacggcattctcgattgctttgctatcgaaggaaagccggatgcggtcgaaactatagcaaatgcttacgtgaagctcggtcgccatcgagaaggtgtcgtgggctttgctcagtgctacctgttcgacgcgcaggacatcgtgacgttcggcgtcacctatcttgagaagcacttcggagccactccgatcgtgccagcacacgaagccgcccagcgctcttgcgagccttccggttag

SEQ ID NO: 168 - DNA, 2415 bp, Escherichia coli
  misc_feature (456)..(456): n is a or c or g or t
atgtcgaattcttatgactcctccagtatcaaagtcctgaaagggctggatgcggtgcgtaagcgcccgggtatgtatatcggcgacacggatgacggcaccggtctgcaccacatggtattcgaggtggtagataacgctatcgacgaagcgctcgcgggtcactgtaaagaaattatcgtcaccattcacgccgataactctgtctctgtacaggatgacgggcgcggcattccgaccggtattcacccggaagagggcgtatcggcggcggaagtgatcatgaccgttctgcacgcaggcggtaaatttgacgataactcctataaagtgtccggcggtctgcacggcgttggtgtttcggtagtaaacgccctgtcgcaaaaactggagctggttatccagcgcgagggtaaaattcaccgtcagatctacgaacacggtgtaccgcaggcnccgctggcggttaccggcgagactgaaaaaaccggcaccatggtgcgtttctggcccagcctcgaaaccttcaccaatgtgaccgagttcgaatatgaaattctggcgaaacgtctgcgtgagttgtcgttcctcaactccggcgtttccattcgtctgcgcgacaagcgcgacggcaaagaagaccacttccactatgaaggcggcatcaaggcgttcgttgaatatctgaacaagaacaaaacgccgatccacccgaatatcttctacttctccactgaaaaagacggtattggcgtcgaagtggcgttgcagtggaacgatggcttccaggaaaacatctactgctttaccaacaacattccgcagcgtgacggcggtactcacctggcaggcttccgtgcggcgatgacccgtaccctgaacgcctacatggacaaagaaggctacagcaaaaaagccaaagtcagcgccaccggtgacgatgcgcgtgaaggcctgattgcggtcgtttccgtgaaagtgccggacccgaaattctcctcccagaccaaagacaaactggtttcttctgaggtgaaatcggcggttgaacagcagatgaacgaactgctggcagaatacctgctggaaaacccaaccgacgcgaaaatcgtggttggcaaaattatcgatgctgcccgtgcccgtgaagcggcgcgtcgcgcgcgtgaaatgacccgccgtaaaggtgcgctcgacttagcgggcctgccgggcaaactggcagactgccaggaacgcgatccggcgctttccgaactgtacctggtggaaggggactccgcgggcggctctgcgaagcaggggcgtaaccgcaagaaccaggcgattctgccgctgaagggtaaaatcctcaacgtcgagaaagcgcgcttcgataagatgctctcttctcaggaagtggcgacgcttatcaccgcgcttggctgtggtatcggtcgtgacgagtacaacccggacaaactgcgttatcacagcatcatcatcatgaccgatgcggacgtcgacggctcgcacattcgtacgctgctgttgaccttcttctatcgtcagatgccggaaatcgttgaacgcggtcacgtctacatcgctcagccgccgctgtacaaagtgaagaaaggcaagcaggaacagtacattaaagacgacgaagcgatggatcagtaccagatctctatcgcgctggacggcgcaacgctgcacaccaacgccagtgcaccggcattggctggcgaagcgttagagaaactggtatctgagtacaacgcgacgcagaaaatgatcaatcgtatggagcgtcgttatccgaaagcaatgctgaaagagcttatctatcagccgacgttgacggaagctgacctttctgatgagcagaccgttacccgctgggtgaacgcgctggtcagcgaactgaacgacaaagaacagcacggcagccagtggaagtttgatgttcacaccaatgctgagcaaaacctgttcgagccgattgttcgcgtgcgtacccacggtgtggatactgactatccgctggatcacgagtttatcaccggtggcgaatatcgtcgtatctgcacgctgggtgagaaactgcgtggcttgctggaagaagatgcgtttatcgaacgtggcgagcgtcgtcagccggtagccagcttcgagcaggcgctggactggctggtgaaagagtcccgtcgcggcctctccatccagcgttataaaggtctgggcgagatgaacccggaacagctgtgggaaaccactatggacccggaaagtcgtcgtatgctgcgcgttaccgttaaagatgcgattgctgccgaccagttgttcaccacgctgatgggcgacgccgttgaaccgcgccgtgcgtttattgaagagaacgccctgaaagcggcgaatatcgatatttaa

SEQ ID NO: 169 - DNA, 645 bp, Escherichia coli
  misc_feature (348)..(348): n is c or t
  misc_feature (456)..(456): n is a or g
atgcgtatcattctgcttggcgctccgggcgcggggaaagggactcaggctcagttcatcatggagaaatatggtattccgcaaatctccactggcgatatgctgcgtgctgcggtcaaatctggctccgagctgggtaaacaagcaaaagacattatggatgctggcaaactggtcaccgacgaactggtgatcgcgctggttaaagagcgcattgctcaggaagactgccgtaatggtttcctgttggacggcttcccgcgtaccattccgcaggcagacgcgatgaaagaagcgggcatcaatgttgattacgttctggaattcgacgtaccggacgaactgatngttgaccgtatcgtcggtcgccgcgttcatgcgccgtctggtcgtgtttatcacgttaaattcaatccgccgaaagtagaaggcaaagacgacgttaccggtgaaganctgactacccgtaaagatgatcaggaagagaccgtacgtaaacgtctggttgaataccatcagatgacagcaccgctgatcggctactactccaaagaagcagaagcgggtaataccaaatacgcgaaagttgacggcaccaagccggttgctgaagttcgcgctgatctggaaaaaatcctcggctaa

SEQ ID NO: 170 - DNA, 1251 bp, Escherichia coli
  misc_feature (243)..(243): n is c or t
  misc_feature (606)..(606): n is a or g or t
atggaaagtaaagtagttgttccggcacaaggcaagaagatcaccctgcaaaacggcaaactcaacgttcctgaaaatccgattatcccttacattgaaggtgatggaatcggtgtagatgtaaccccagccatgctgaaagtggtcgacgctgcagtcgagaaagcctataaaggcgagcgtaaaatctcctggatggaaatttacaccggtgaaaaatccacacaggtttatggtcaggangtctggctgcctgctgaaactcttgatctgattcgtgaatatcgcgttgccattaaaggtccgctgaccactccggttggtggcggtattcgctctctgaacgttgccctgcgccaggaactggatctctacatctgcctgcgtccggtacgttactatcagggcactccaagcccggttaaacaccctgaactgaccgatatggttatcttccgtgaaaactcggaagacatttatgcgggtatcgaatggaaagcagactctgccgacgccgagaaagtgattaaattcctgcgtgaagagatgggggtgaagaaaattcgcttcccggaacattgtggtatcggtattaagccgtgttcngaagaaggcaccaaacgtctggttcgtgcagcgatcgaatacgcaattgctaacgatcgtgactctgtgactctggtgcacaaaggcaacatcatgaagttcaccgaaggagcgtttaaagactggggctaccagctggcgcgtgaagagtttggcggtgaactgatcgacggtggcccgtggctgaaagttaaaaacccgaacactggcaaagagatcgtcattaaagacgtgattgctgatgcattcctgcaacagatcctgctgcgtccggctgaatatgatgttatcgcctgtatgaacctgaacggtgactacatttctgacgccctggcagcgcaggttggcggtatcggtatcgcccctggtgcaaacatcggtgacgaatgcgccctgtttgaagccacccacggtactgcgccgaaatatgccggtcaggacaaagtaaatcctggctctattattctctccgctgagatgatgctgcgccacatgggttggaccgaagcggctgacttaattgttaaaggtatggaaggcgcaatcaacgcgaaaaccgtaacctatgacttcgagcgtctgatggatggcgctaaactgctgaaatgttcagagtttggtgacgcgatcatcgaaaacatgtaa

SEQ ID NO: 171 - DNA, 1299 bp, Escherichia coli
  misc_feature (426)..(426): n is a or c or g or t
atgggtaacaacgtcgtcgtactgggcacccaatggggtgacgaaggtaaaggtaagatcgtcgatcttctgactgaacgggctaaatatgttgtacgctaccagggcggtcacaacgcaggccatactctcgtaatcaacggtgaaaaaaccgttctccatcttattccatcaggtattctccgcgagaatgtaaccagcatcatcggtaacggtgttgtgctgtctccggccgcgctgatgaaagagatgaaagaactggaagaccgtggcatccccgttcgtgagcgtctgctgctgtctgaagcatgtccgctgatccttgattatcacgttgcgctggataacgcgcgtgagaaagcgcgtggcgcgaaagcgatcggcaccaccggtcgtggtatcgggcctgcttatgaagataaagtngcacgtcgcggtctgcgtgttggcgaccttttcgacaaagaaaccttcgctgaaaaactgaaagaagtgatggaatatcacaacttccagttggttaactactacaaagctgaagcggttgattaccagaaagttctggatgatacgatggctgttgccgacatcctgacttctatggtggttgacgtttctgacctgctcgaccaggcgcgtcagcgtggcgatttcgtcatgtttgaaggtgcgcagggtacgctgctggatatcgaccacggtacttatccgtacgtaacttcttccaacaccactgctggtggcgtggcgaccggttccggcctgggcccgcgttatgttgattacgttctgggtatcctcaaagcttactccactcgtgtaggtgcaggtccgttcccgaccgaactgtttgatgaaactggcgagttcctctgcaagcagggtaacgaattcggcgcaactacggggcgtcgtcgtcgtaccggctggctggacaccgttgccgttcgtcgtgcggtacagctgaactccctgtctggcttctgcctgactaaactggacgttctggatggcctgaaagaggttaaactctgcgtggcttaccgtatgccggatggtcgcgaagtgactaccactccgctggcagctgacgactggaaaggtgtagagccgatttacgaaaccatgccgggctggtctgaatccaccttcggcgtgaaagatcgtagcggcctgccgcaggcggcgctgaactatatcaagcgtattgaagagctgactggtgtgccgatcgatatcatctctaccggtccggatcgtactgaaaccatgattctgcgcgacccgttcgacgcgtaa

SEQ ID NO: 172 - DNA, 609 bp, Escherichia coli
  misc_feature (384)..(384): n is c or t
atgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccacttctggcgcaacagcatattgaaggtcattatcaggtcgatccttccttattcaagccgaatgctgatttcctgctgcgcgtcagcgggatgtcgatgaaagatatcggcattatggatggngacttgctggcagtgcataaaactcaggatgtacgtaacggtcaggtcgttgtcgcacgtattgatgacgaagttaccgttaagcgcctgaaaaaacagggcaataaagtcgaactgttgccagaaaatagcgagtttaaaccaattgtcgttgaccttcgtcagcagagcttcaccattgaagggctggcggttggggttattcgcaacggcgactggctgtaa

SEQ ID NO: 173 - DNA, 855 bp, Escherichia coli
  misc_feature (279)..(279): n is c or t
atgactgacaaaatgcaaagtttagctttagccccagttggcaacctggattcctacatccgggcagctaacgcgtggccgatgttgtcggctgacgaggagcgggcgctggctgaaaagctgcattaccatggcgatctggaagcagctaaaacgctgatcctgtctcacctgcggtttgttgttcatattgctcgtaattatgcgggctatggcctgccacaggcggatttgattcaggaaggtaacatcggcctgatgaaagcagtgcgccgtttnaacccggaagtgggtgtgcgcctggtctccttcgccgttcactggatcaaagcagagatccacgaatacgttctgcgtaactggcgtatcgtcaaagttgcgaccaccaaagcgcagcgcaaactgttcttcaacctgcgtaaaaccaagcagcgtctgggctggtttaaccaggatgaagtcgaaatggtggcccgtgaactgggcgtaaccagcaaagacgtacgtgagatggaatcacgtatggcggcacaggacatgacctttgacctgtcttccgacgacgattccgacagccagccgatggctccggtgctctatctgcaggataaatcatctaactttgccgacggcattgaagatgataactgggaagagcaggcggcaaaccgtctgaccgacgcgatgcagggtctggacgaacgcagccaggacatcatccgtgcgcgctggctggacgaagacaacaagtccacgttgcaggaactggctgaccgttacggcgtttccgctgagcgtgtacgccagctggaaaagaacgcgatgaaaaaattgcgtgctgccattgaagcgtaa